SEQUENCE LISTING (44 sequences)

SEQ ID NO: 1 - protein, 8 aa, synthetic construct
GFTFSIYG

SEQ ID NO: 2 - protein, 8 aa, synthetic construct
MSFDGKTK

SEQ ID NO: 3 - protein, 12 aa, synthetic construct
AKGGATAAVFDS

SEQ ID NO: 4 - protein, 12 aa, synthetic construct
QSLLYRSNSKNY

SEQ ID NO: 5 - skipped

SEQ ID NO: 6 - protein, 9 aa, synthetic construct
QQYYNPPWT

SEQ ID NO: 7 - protein, 119 aa, Homo sapiens
QVQLVESGGGMVQPGRSLRLSCAASGFTFSIYGMHWVRQAPGRGLEWVAVMSFDGKTKYYADSVKGRFTISRDNSKNTLYLQMNSLRRVDTAVYYCAKGGATAAVFDSWGQGTLVTVSS

SEQ ID NO: 8 - protein, 113 aa, Homo sapiens
DIVMTQSPDSLAVSLGERAAINCKSSQSLLYRSNSKNYLAWYQQQPGQPPKLLVYWASTRESGVPDRFSGSGSGTDFTLTISSLQAEDVAVYYCQQYYNPPWTFGRGTKVEIK

SEQ ID NO: 9 - protein, 12 aa, synthetic construct
QSLLYRSNNKNY

SEQ ID NO: 10 - protein, 118 aa, synthetic construct
QVQLVESGGGVVQPGRSLRLSCAASGFTFSIYGMHWVRQAPGKGLEWVAVMSFDGKTKYYADSVKGRFTISRDNSKNTLYLQMNSLRAEDTAVYYCAKGGATAAVFDSWGQGTLVTVS

SEQ ID NO: 11 - protein, 113 aa, synthetic construct
DIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNPPWTFGQGTKVEIK

SEQ ID NO: 12 - protein, 8 aa, synthetic construct
GFTFSNYG

SEQ ID NO: 13 - protein, 8 aa, synthetic construct
TSFDGKTK

SEQ ID NO: 14 - protein, 12 aa, synthetic construct
QSLLYRRNNKNY

SEQ ID NO: 15 - protein, 119 aa, synthetic construct
QVQLVESGGGVVQPGRSLRLSCAASGFTFSNYGMHWVRQAPGKGLEWVAVTSFDGKTKYYADSVKGRFTISRDNSKNTLYLQMNSLRAEDTAVYYCAKGGATAAVFDSWGQGTLVTVSS

SEQ ID NO: 16 - protein, 113 aa, synthetic construct
DIVMTQSPDSLAVSLGERATINCKSSQSLLYRRNNKNYLAWYQQKPGQPPKLLIYWASTRESGVPDRFSGSGSGTDFTLTISSLQAEDVAVYYCQQYYNPPWTFGQGTKVEIK

SEQ ID NO: 17 - protein, 12 aa, synthetic construct
AKGGATAAVLDS

SEQ ID NO: 18 - protein, 119 aa, synthetic construct
QVQLVESGGGVVQPGRSLRLSCAASGFTFSNYGMHWVRQAPGKGLEWVAVTSFDGKTKYYADSVKGRFTISRDNSKNTLYLQMNSLRAEDTAVYYCAKGGATAAVLDSWGQGTLVTVSS

SEQ ID NO: 19 - protein, 113 aa, synthetic construct; identical to SEQ ID NO: 16

SEQ ID NO: 20 - protein, 449 aa, synthetic construct
QVQLVESGGGVVQPGRSLRLSCAASGFTFSNYGMHWVRQAPGKGLEWVAVTSFDGKTKYYADSVKGRFTISRDNSKNTLYLQMNSLRAEDTAVYYCAKGGATAAVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - protein, 220 aa, synthetic construct
DIVMTQSPDSLAVSLGERATINCKSSQSLLYRRNNKNYLAWYQQKPGQPPKLLIYWASTRESGVPDRFSGSGSGTDFTLTISSLQAEDVAVYYCQQYYNPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 22 - protein, 449 aa, synthetic construct
QVQLVESGGGVVQPGRSLRLSCAASGFTFSNYGMHWVRQAPGKGLEWVAVTSFDGKTKYYADSVKGRFTISRDNSKNTLYLQMNSLRAEDTAVYYCAKGGATAAVLDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 220 aa, synthetic construct; identical to SEQ ID NO: 21

SEQ ID NO: 24 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 25 - protein, 59 aa, synthetic construct
GKCGPPPPIDNGDITSFPLSVYAPASSVEYQCQNLYQLEGNKRITCRNGQWSEPPKCLH

SEQ ID NO: 26 - protein, 58 aa, synthetic construct
KCGPPPPIDNGDITSFPLSVYAPASSVEYQCQNLYQLEGNKRITCRNGQWSEPPKCLH

SEQ ID NO: 27 - protein, 119 aa, synthetic construct
EVQLVESGGGVVQPGRSLRLSCAASGFTFSRYGMHWVRQAPGKGLEWVAVMSYDGSTKYYADSVKGRFAISRDNPKNTLFLQMNSLRPDDTAVYYCAKGGAAAAVMDVWGKGTTVTVSS

SEQ ID NO: 28 - protein, 113 aa, synthetic construct
DIVMTQSPDSLTVSLGERATISCKSSQRLLYSSNNKNYLAWYQQKPGQPPKLLMYWASTRESGVPDRFSGSGSGTDFSLTISSLQAEDVAVYYCQQYYNPPWTFGQGTKVEVK

SEQ ID NO: 29 - protein, 14 aa, synthetic construct
CAKGGATAAVFDSW

SEQ ID NO: 30 - protein, 14 aa, synthetic construct
CAKGGAAAAVFDSW

SEQ ID NO: 31 - protein, 14 aa, synthetic construct
CAKGAGAAAVFDHW

SEQ ID NO: 32 - DNA, 1426 bp, synthetic construct
  source 1..1426: mol_type = other DNA
aagcttgccgccaccatgggctggtcctgcatcatcctgtttctggtggctaccgctaccggcgtgcactctcaggttcagctggttgaatctggcggcggagtggtgcagcctggcagatctctgagactgtcttgtgccgcctccggcttcaccttctccaactacggaatgcactgggtccgacaggcccctggcaaaggattggaatgggtcgccgtgacctccttcgacggcaagaccaagtactacgccgactccgtgaagggcagattcaccatctctcgggacaactccaagaacaccctgtacctgcagatgaactccctgagagccgaggacaccgccgtgtactactgtgctaaaggcggcgctacagccgccgtgttggattcttggggacagggcacactggtcaccgtgtcctctgcttctaccaagggacccagcgtgttccctctggctccttccagcaagtctacctctggcggaacagctgctctgggctgcctggtcaaggactactttcctgagcctgtgaccgtgtcttggaactctggcgctctgacatctggcgtgcacacctttccagctgtgctgcagtcctccggcctgtactctctgtcctctgtcgtgaccgtgccttccagctctctgggaacccagacctacatctgcaatgtgaaccacaagccttccaacaccaaggtggacaagaaggtggaacccaagtcctgcgacaagacccacacctgtcctccatgtcctgctccagaactgctcggcggaccttccgtgttcctgtttcctccaaagcctaaggacaccctgatgatctctcggacccctgaagtgacctgcgtggtggtggatgtgtctcacgaggacccagaagtgaagttcaattggtacgtggacggcgtggaagtgcacaacgctaagaccaagcctagagaggaacagtacaactccacctacagagtggtgtccgtgctgaccgtgctgcaccaggattggctgaacggcaaagagtacaagtgcaaggtgtccaacaaggccctgcctgctcctatcgaaaagaccatctccaaggccaagggccagcctagggaaccccaggtttacaccttgcctccatctcgggacgagctgaccaagaaccaggtgtccctgacctgcctcgtgaagggattctacccctccgatatcgccgtggaatgggagtctaatggccagcctgagaacaactacaagacaacccctcctgtgctggactccgacggctcattcttcctgtactccaagctgacagtggacaagtccagatggcagcagggcaacgtgttctcctgctccgtgatgcacgaggccctgcacaatcactacacacagaagtccctgtctctgtcccctggctgatgaattc

SEQ ID NO: 33 - DNA, 742 bp, synthetic construct
  source 1..742: mol_type = other DNA
aagcttgccgccaccatgggctggtcctgcatcatcctgtttctggtggctaccgctaccggcgtgcactccgatatcgtgatgacccagtctcctgacagcctggctgtgtctctgggcgagagagccaccatcaactgcaagtcctctcagtccctgctgtaccggcggaacaacaagaactacctggcctggtatcagcagaagcccggccagcctcctaagctgctgatctactgggcctccaccagagaatctggcgtgcccgatagattctccggctctggctctggcaccgactttaccctgacaatcagctccctgcaggccgaggatgtggccgtgtactactgccagcagtactacaaccctccttggacctttggccagggcaccaaggtggaaatcaagcggacagtggccgctccttccgtgttcatcttcccaccttccgacgagcagctgaagtccggcacagcttctgtcgtgtgcctgctgaacaacttctaccctcgggaagccaaggtgcagtggaaggtggacaatgccctgcagtccggcaactcccaagagtctgtgaccgagcaggactccaaggacagcacctacagcctgtcctccacactgaccctgtccaaggccgactacgagaagcacaaggtgtacgcctgcgaagtgacccatcagggcctgtctagccctgtgaccaagtctttcaaccggggcgagtgctgatgaattc

SEQ ID NO: 34 - DNA, 1407 bp, synthetic construct
  source 1..1407: mol_type = other DNA
atgggctggtcctgcatcatcctgttcctggtggctaccgccaccggcgtgcactcccaagtgcaactagtggaaagtggtggtggtgtggtgcaacccggacgctccctccggctgtcttgtgctgcttctggcttcaccttctccaactacggcatgcactgggtcagacaggcccctggaaaaggcctggaatgggtggccgtgacctccttcgatggcaagaccaagtactatgccgactccgtgaagggcagattcaccatctcccgggacaactccaagaacaccctgtacctgcagatgaactccctgagagccgaggacaccgctgtgtactactgcgccaagggcggcgctaccgccgccgtgttcgactcttggggccagggcacactggtgaccgtgtccagcgcaagtactaagggtccaagtgtgtttccactggctccctctagcaagtccacctctggcggcaccgccgccctgggctgcctggtcaaggactacttccccgagcccgtcaccgtgtcctggaactccggcgctctgacctccggcgtgcataccttccctgctgtgctgcagtcttctggcctgtactccctgtcctccgtcgtgacagtgccttctagctctctcggcacacagacatacatctgcaatgtgaaccacaagccttccaacaccaaagtggataagaaggtggaacctaagtcctgcgacaaaacccacacctgtcctccttgccctgctcctgaagccgctggcggaccttctgtgtttctgtttcccccaaagcccaaagacaccctgatgatctctcggacacctgaggtgacctgcgtggtggtggatgtgtctcacgaggaccccgaagtgaagttcaactggtacgtggacggcgtggaagtgcacaacgccaagaccaagccacgcgaagagcagtacaactctacctacagagtggtgtccgtgctcacagtgctgcaccaggactggctgaacggaaaagagtacaagtgcaaggtctctaacaaggctctgccagcccctatcgagaagaccatctccaaggccaagggccaacctagagagcctcaagtgtacaccctgcctcctagccgggacgagctgaccaagaaccaggtttccctgacctgtctggtgaaaggcttctacccctccgatatcgccgtggaatgggagtccaacggccagcctgagaacaactacaagaccacccctcctgtgctggactccgatggatccttcttcctgtatagcaagctgaccgtggacaagtccagatggcagcagggcaacgtgttcagctgttccgtgatgcacgaggccctgcataatcactacacccagaagtctctgagcctgtctcctggcaagtga

SEQ ID NO: 35 - DNA, 720 bp, synthetic construct
  source 1..720: mol_type = other DNA
atgggctggtcctgcatcatcctgttcctggtggctaccgccaccggcgtgcactccgatatcgtgatgactcaaagtccagatagtctcgctgtgtccctgggcgagagagccacaatcaactgcaagtccagccaaagcctgctgtaccggcggaacaacaagaactacctggcctggtaccagcagaaacctggccagcctcctaagctgctgatctactgggcttctaccagagagtctggcgtcccagatagattctccggctctggctccggaaccgacttcaccctgaccatctcctctctgcaggccgaggacgtggccgtgtactactgtcagcagtactataatcccccttggacctttggccagggcaccaaggtggaaatcaagagaaccgtggccgctccttctgtgttcatcttccctccttccgacgagcagctgaagtccggcaccgcttccgtggtgtgcctgctgaacaacttctaccctagagaggccaaggtgcagtggaaggtggacaacgccctgcagtctggcaactcccaagagtccgtcaccgagcaggactccaaggactctacctactccctgtcctccaccctgacactgagcaaggccgactacgagaagcacaaggtgtacgcctgcgaggtgacccaccagggcctgtcttctcctgtgaccaagtccttcaacagaggcgagtgctga

SEQ ID NO: 36 - protein, 449 aa, synthetic construct
QVQLVESGGGMVQPGRSLRLSCAASGFTFSIYGMHWVRQAPGRGLEWVAVMSFDGKTKYYADSVKGRFTISRDNSKNTLYLQMNSLRRVDTAVYYCAKGGATAAVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 220 aa, synthetic construct
DIVMTQSPDSLAVSLGERAAINCKSSQSLLYRSNSKNYLAWYQQQPGQPPKLLVYWASTRESGVPDRFSGSGSGTDFTLTISSLQAEDVAVYYCQQYYNPPWTFGRGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 449 aa, synthetic construct
QVQLVESGGGVVQPGRSLRLSCAASGFTFSIYGMHWVRQAPGKGLEWVAVMSFDGKTKYYADSVKGRFTISRDNSKNTLYLQMNSLRAEDTAVYYCAKGGATAAVFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 220 aa, synthetic construct
DIVMTQSPDSLAVSLGERATINCKSSQSLLYRSNSKNYLAWYQQKPGQPPKLLIYWASTRESGVPDRFSGSGSGTDFTLTISSLQAEDVAVYYCQQYYNPPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 449 aa, synthetic construct
EVQLVESGGGVVQPGRSLRLSCAASGFTFSRYGMHWVRQAPGKGLEWVAVMSYDGSTKYYADSVKGRFAISRDNPKNTLFLQMNSLRPDDTAVYYCAKGGAAAAVMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 41 - protein, 220 aa, synthetic construct
DIVMTQSPDSLTVSLGERATISCKSSQRLLYSSNNKNYLAWYQQKPGQPPKLLMYWASTRESGVPDRFSGSGSGTDFSLTISSLQAEDVAVYYCQQYYNPPWTFGQGTKVEVKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 246 aa, synthetic construct
QVQLVESGGGVVQPGRSLRLSCAASGFTFSIYGMHWVRQAPGKGLEWVAVMSFDGKTKYYADSVKGRFTISRDNSKNTLYLQMNSLRAEDTAVYYCAKGGATAAVFDSWGQGTLVTVSGGGGSGGGGSGGGGSDIVMTQSPDSLAVSLGERATINCKSSQSLLYRSNNKNYLAWYQQKPGQPPKLLIYWASTRESGVPDRFSGSGSGTDFTLTISSLQAEDVAVYYCQQYYNPPWTFGQGTKVEIK

SEQ ID NO: 43 - DNA, 741 bp, synthetic construct
  source 1..741: mol_type = other DNA
caggttcagctggttgaaagcggtggtggtgttgttcagcctggtcgtagcctgcgtctgagctgtgcagcaagcggttttacctttagcatttatggtatgcattgggttcgtcaggcacctggtaaaggtctggaatgggttgcagttatgagctttgatggtaaaaccaaatattacgccgatagcgtgaaaggtcgctttaccattagccgtgataatagcaaaaataccctgtacctgcagatgaatagtctgcgtgcagaggataccgcagtgtattattgtgcaaaaggtggtgcaaccgcagcagtttttgatagctggggtcagggcaccctggttaccgttagcagcggtggcggtggtagtggtggtggcggtagcggaggtggtggttcagatattgttatgacccagagtccggatagcctggcagttagcctgggtgaacgtgcaaccattaattgtaaaagcagccagagcctgctgtatcgtagcaataacaaaaactatctggcatggtatcagcagaaaccgggtcagcctccgaaactgctgatttattgggcaagcacccgtgaaagtggtgttccggatcgttttagcggtagtggtagcggcaccgattttaccctgaccattagcagtctgcaggcagaggacgttgcagtctattactgtcagcagtattacaatccgccttggacctttggccagggcaccaaagttgaaatcaaa

SEQ ID NO: 44 - protein, 119 aa, synthetic construct
QVQLVESGGGVVQPGRSLRLSCAASGFTFSIYGMHWVRQAPGKGLEWVAVMSFDGKTKYYADSVKGRFTISRDNSKNTLYLQMNSLRAEDTAVYYCAKGGATAAVFDSWGQGTLVTVSS

(19) United States
(12) Patent Application Publication
(10) Pub. No.: US 2025/0304666 A1
(43) Pub. Date: Oct. 2, 2025

(54) ANTI-COMPLEMENT FACTOR H ANTIBODIES

(71) Applicant: Alchemab Therapeutics Ltd, London (GB)

(72) Inventors: Ralph Raymond Minter, Cambridge (GB); Donna Kirsty Finch, Cambridge (GB); Jacob Daniel Galson, Cambridge (GB); Sandrine Legg, Cambridge (GB); Danielle Hayley Minns, Cambridge (GB); Matthew Kraman, Cambridge (GB); Jane Osbourn, Cambridge (GB)

(73) Assignee: Alchemab Therapeutics Ltd, London (GB)

(21) Appl. No.: 18/866,681

(22) PCT Filed: May 17, 2023
(86) PCT No.: PCT/EP2023/063380

§ 371 Date: Nov. 18, 2024

(30) Foreign Application Priority Data
May 17, 2022 (GB) 2207219.3
Jun. 10, 2022 (GB) 2208525.2
Oct. 20, 2022 (GB) 2215527.9

(51) Int. Cl. C07K 16/18; A61K 39/00; A61K 45/06; A61P 35/00

(52) U.S. Cl. CPC C07K 16/18; A61K 45/06; A61P 35/00; A61K 2039/505; C07K 2317/33; C07K 2317/34; C07K 2317/565; C07K 2317/76; C07K 2317/94

(57) ABSTRACT

Antibodies and fragments thereof capable of binding to Complement Factor H are described. Related products and therapeutic uses are also described.

### FIELD OF THE INVENTION

The present invention relates to antibodies and fragments thereof capable of binding to Complement Factor H, and particularly, although not exclusively, to improved therapeutic antibodies.

### BACKGROUND

The complement system is a key component of innate immunity, consisting of a domino-like cascade of over 50 proteins (Ricklin et al, 2010). Complement activation results in the opsonisation of pathogenic particles or diseased cells for destruction via phagocytosis or cell lysis. Activation occurs through three pathways: classical, lectin and alternative, all of which converge in the cleavage of central component C3 by C3 convertase. This produces C3b, which covalently bind the activating surface of a pathogen or neoplasm and, through the action of several other components, forms bimolecular C3bBb, a C3 convertase utilised by the complement “alternative pathway”. The formation of this amplification loop is moderated by several mediators of the complement system (Schmidt et al 2016).

Complement Factor H is a key negative moderator of the complement system, and especially the alternative pathway. It is found free circulating in plasma and bound to cell surface. CFH suppresses complement activation by competing for C3b binding (Kazatchkine et al, 1979), by accelerating the decay of surface bound C3 and C5 convertases (Weiler et al, 1976), and by acting as a cofactor to facilitate cleavage of C3b into an inactive form (Harrison & Lachmann, 1980). Surface bound CFH effectively protects against alternative pathway activity. CFH has also been reported to bind to complement receptors such as CR3 and has immunomodulatory effects on a range of immune cells suggesting CFH might have a direct anti-inflammatory and tolerogenic effect towards infiltrated leukocytes (Parente et al 2017). CFH recruitment is well reported as a mechanism through which pathogens and cancers evade complement activation (Moore et al, 2021). Given the wide-ranging effects of the complement pathway, and the negative consequences associated with off-target effects and over-activation of the system and inflammation, CFH remains an elusive drug target. There remains a need for improved anti-CFH antibodies.

The present invention has been devised in light of the above considerations.

### SUMMARY OF THE INVENTION

The present invention concerns a broadly tolerated, stable and effective anti-CFH antibody. In contrast to traditional antibody generation programs, where antibodies are raised against specific antigens and reviewed for activity and tolerability in vivo, the anti-CFH antibodies herein were generated starting from an analysis of broadly tolerated antibodies and working backwards through their characterisation to identify targets. The antibodies of the invention were identified through an analysis of antibody repertoire data in order to identify convergent sequence clusters amongst both viral (Ehrhardt et al 2019 Nat Med 25:1589, Davis et al 2019 Cell 177:1566, Bowers et al 2014 PLOSOne 9: e81913), and tumour (PDAC, Prostate and Melanoma) cohorts. A representative heavy chain from this cluster was paired with an appropriate light chain and expressed in IgG1 format as ATL4717. Target identification revealed CFH, and CFH-related proteins as potential antigens. As this antibody cluster is convergently present in various disease state patient groups, it is tolerated in vivo and off-target or side effects are minimised.

The antibodies of the invention were produced by further developing antibodies from the identified cluster so as to improve desirable properties not limited to increased thermal stability, specificity for cell-associated CFH, and immune response modification. In addition, yields of the original antibody ATL4717 in IgG1 format were extremely low in transient HEK293 cultures and required improvement to be compatible with manufacturing. These steps go beyond routine optimisation and required analysis of the cohort diversity, extensive testing, investigation of multiple beneficial and mechanistic properties simultaneously, and guided engineering to produce antibodies not found in naturally occurring populations. These novel antibodies with improved properties include ATL5170 and 5155, as disclosed herein.

In a first aspect, the invention relates to an isolated antibody or antibody fragment thereof which specifically binds to Complement Factor H (CFH) protein or a fragment thereof. In some embodiments, the antibody comprises:
a. a heavy chain variable domain (VH) with the following CDRs:
   i. HCDR1 comprising amino acid sequence SEQ ID NO:12,
   ii. HCDR2 comprising amino acid sequence SEQ ID NO:13, and
   iii. HCDR3 comprising amino acid sequence SEQ ID NO:17; and
b. a light chain variable domain (VL) with the following CDRs:
   i. LCDR1 comprising amino acid sequence SEQ ID NO:14,
   ii. LCDR2 comprising amino acid sequence SEQ ID NO:5, and
   iii. LCDR3 comprising amino acid sequence SEQ ID NO:6.

In some embodiments, the isolated antibody or antibody heavy chain variable domain comprises amino acid sequence SEQ ID NO: 18, and/or the light chain variable domain comprises amino acid sequence SEQ ID NO:19. Preferably, the heavy chain comprises SEQ ID NO:22, and/or the light chain comprises SEQ ID NO:23. A representative antibody comprising a heavy chain of SEQ ID NO:22, and a light chain SEQ ID NO:23 is referred to herein as “ATL5170”.

In other embodiments, the antibody comprises:
a. heavy chain variable domain (VH) with the following CDRs:
   i. HCDR1 comprising amino acid sequence SEQ ID NO:12,
   ii. HCDR2 comprising amino acid sequence SEQ ID NO:13, and
   iii. HCDR3 comprising amino acid sequence SEQ ID NO:3; and
b. a light chain variable domain (VL) with the following CDRs:
   i. LCDR1 comprising amino acid sequence SEQ ID NO:14,
   ii. LCDR2 comprising amino acid sequence SEQ ID NO:5, and
   iii. LCDR3 comprising amino acid sequence SEQ ID NO:6.

In other embodiments, the heavy chain variable domain comprises amino acid sequence SEQ ID NO:15, and/or the light chain variable domain comprises amino acid sequence SEQ ID NO:16. Preferably, the heavy chain comprises SEQ ID NO:20, and/or the light chain comprises SEQ ID NO:21. A representative antibody comprising a heavy chain of SEQ ID NO:20, and a light chain SEQ ID NO:21 is referred to herein as “ATL5155”.

In some embodiments of the first aspect, the antibody comprises one or more framework substitutions. The substitutions may be selected from L50P, S70G, and L123Q within the VH domain, and/or L11Q and E68V within the VL domain.

In some embodiments, the isolated antibody or antibody fragment thereof according to any previous claim, wherein the antibody is IgG1.

In a further aspect, the invention provides a method of treating a disease or disorder, comprising administering an effective amount of an isolated antibody or antibody fragment thereof according to the invention. In a related aspect, the invention provides the use of an isolated antibody or antibody fragment thereof according to the invention in the manufacture of a medicament for the treatment of a disease or disorder. In a related aspect, the invention provides a composition comprising an isolated antibody or antibody fragment thereof according to the first aspect, for use in the treatment of a disease or disorder, e.g. through administering an effective amount of the composition to the subject. In these aspects, the disease or disorder is preferably selected from cancer or an infectious disease or disorder.

In a further aspect, the invention provides a method of increasing complement dependent lysis of a cell, in vitro or in vivo, comprising contacting the cell with an antibody or antibody fragment thereof according to the invention.

In a further aspect, the invention provides an in vitro or in vivo method of increasing C3 deposition, preferably C3b and/or C3d deposition, on a cell, comprising contacting the cell with an antibody or antibody fragment thereof according to the invention.

In a further aspect, the invention provides an in vitro or in vivo method of inhibiting CFH binding to C3b in a subject or a cell, comprising contacting the cell with an antibody or antibody fragment thereof according to the invention.

In a further aspect, the invention provides an in vitro or in vivo method of activating an immune cell, comprising contacting the cell with an antibody or antibody fragment thereof according to the invention. Immune activation may be mediated via increased C3b/C3d deposition on cells and/or activation of CFH-interacting molecules or receptors at the cell surface.

The invention further provides a method of detecting CFH, in vitro or in vivo, comprising contacting a sample with an antibody or antibody fragment thereof according to the invention, and detecting antibody binding.

The invention further provides a DNA molecule or set of DNA molecules encoding an antibody or antibody fragment thereof according to the invention, a vector or set of vectors encoding said DNA molecule or molecules, and a host cell comprising said vector or set of vectors.

Also disclosed is an isolated antibody or antibody fragment thereof which specifically binds to Complement Factor H (CFH) protein or a fragment thereof, the antibody comprising:
a. a heavy chain variable domain (VH) with the following CDRs:
   i. HCDR1 comprising amino acid sequence SEQ ID NO:1,
   ii. HCDR2 comprising amino acid sequence SEQ ID NO:2,
   iii. HCDR3 comprising amino acid sequence SEQ ID NO:3; and
b. a light chain variable domain (VL) with the following CDRs:
   i. LCDR1 comprising amino acid sequence SEQ ID NO:4,
   ii. LCDR2 comprising amino acid sequence SEQ ID NO:5, and
   iii. LCDR3 comprising amino acid sequence SEQ ID NO:6.

The antibody or fragment thereof thus disclosed may comprise a heavy chain variable domain comprising amino acid sequence SEQ ID NO:7, and/or a light chain variable domain comprising amino acid sequence SEQ ID NO:8. A representative antibody comprising these sequences as disclosed herein is ATL4177.

Also disclosed is an isolated antibody or antibody fragment thereof which specifically binds to Complement Factor H (CFH) protein or a fragment thereof, the antibody comprising:
a. A heavy chain variable domain (VH) with the following CDRs:
   i. HCDR1 comprising amino acid sequence SEQ ID NO:1,
   ii. HCDR2 comprising amino acid sequence SEQ ID NO:2, and
   iii. HCDR3 comprising amino acid sequence SEQ ID NO:3; and
b. a light chain variable domain (VL) with the following CDRs:
   i. LCDR1 comprising amino acid sequence SEQ ID NO:9,
   ii. LCDR2 comprising amino acid sequence SEQ ID NO:5, and
   iii. LCDR3 comprising amino acid sequence SEQ ID NO:6.

The antibody or fragment thereof thus disclosed may comprise a heavy chain variable domain comprising amino acid sequence SEQ ID NO:10, and/or a light chain variable domain comprising amino acid sequence SEQ ID NO:11. A representative antibody comprising these sequences as disclosed herein is ATL4894.

Also provided is a method of predicting whether a subject will respond to treatment with a CFH inhibitor. The method comprises:
(i) obtaining or providing a sample of a tumour from the subject, and
(ii) determining the level of CFH expression, mutational burden and/or immune cell infiltration within said sample.

The CFH inhibitor is preferably an antibody or antibody fragment thereof which specifically binds to CFH protein or a fragment thereof. In some embodiments, the CFH inhibitor is an antibody or antibody fragment thereof as defined herein, for example an antibody or fragment thereof according to the first aspect and/or according to Table 1.

In some embodiments, the subject is predicted to respond to the treatment if they are determined to have high or elevated CFH expression, mutational burden and/or immune cell infiltration.

In some embodiments, the method further comprises selecting the subject for treatment with a CFH inhibitor, and/or administering the CFH inhibitor to the subject. Optionally, an immune checkpoint inhibitor may also be administered.

Also provided is a method of selecting a subject for treatment with a CFH inhibitor. The method comprises:
(i) obtaining or providing a sample of a tumour from the subject, and
(ii) determining the level of CFH expression, mutational burden and/or immune cell infiltration within said sample.

The CFH inhibitor is preferably an antibody or antibody fragment thereof which specifically binds to CFH protein or a fragment thereof. In some embodiments, the CFH inhibitor is an antibody or antibody fragment thereof as defined herein, for example an antibody or fragment according to the first aspect and/or according to Table 1.

In some embodiments, the subject is selected for treatment if they are determined to have a high or elevated level of CFH expression, mutational burden and/or immune cell infiltration.

In some embodiments, the method further comprises administering the CFH inhibitor to the subject. Optionally, an immune checkpoint inhibitor may also be administered.

Administration of an immune checkpoint inhibitor may be performed before, after or concurrently with the CFH inhibitor.

Administration of a CFH inhibitor and/or isolated antibody or fragment thereof or composition comprising an isolated antibody or fragment thereof may be in a therapeutically effective amount.

High or elevated level of CFH expression, mutational burden and/or immune cell infiltration may be determined relative to a control level. A control level may correspond to a level in a normal individual or normal population of individuals, a corresponding level in another tumour, tumour model or population of tumours.

Also disclosed are kits comprising the antibodies or fragments thereof provided herein, optionally in combination with one or more excipient, carrier, diluent, further active agent, or instruction manual.

The invention includes the combination of the aspects and preferred features described except where such a combination is clearly impermissible or expressly avoided.

### SUMMARY OF THE FIGURES

Embodiments and experiments illustrating the principles of the invention will now be discussed with reference to the accompanying figures in which:

FIG. 1—Antibody discovery workflow: identification of ATL4717

FIG. 2—ATL4717 binds to CFH. Results of ELISAs measuring reactivity of ATL4717 to reduced (A) and non-reduced CFH (B). Optical density at 450 nm (OD 450 nm) for the indicated concentrations. (C-E) Results of ELISAs measuring binding of ATL4717 (or commercial anti-CFH (green)/isotype (red)) to recombinant human CFH (C), recombinant mouse CFH (D), and lysozyme (E).

FIG. 3—ATL4717 increases tumour cell killing. (A) A 3D spheroid culture of the A549 tumour cell line was either untreated (blue), treated with serum (orange), or treated with the ATL4717 at day 4. Time is indicated in days. (B) Quantification of spheroid size 5 days after treatment.

FIG. 4—ATL4717 increases monocyte survival. Graph shows percentage dead cells (Cytotox green) of monocytes (x-axis) over time.

FIG. 5—ATL4717 alters monocyte cytokine production. Monocytes were left untreated (top) or stimulated with LPS (bottom), and the concentrations of pro-inflammatory cytokines MCP-1, IL-8, and TNF-alpha, and the anti-inflammatory cytokine IL-10 were determined in the presence and absence of CFH, ATL4717 or isotype control.

FIG. 6—ATL4717 drives infiltration of CD11b+ cells and increases C3d deposition in B16/F10 tumours in a syngeneic mouse model. (A) Confocal image of CD11b+ (green) in B16/F10 tumours. (B) Quantification of DAPI+ (live) CD11b+ cells in ATL4717-treated mice versus IgG controls. (C) Confocal image of C3d deposition (green) in B16/F10 tumours. (D) Quantification of DAPI+ (live) C3d+ cells in ATL4717-treated mice versus IgG controls.

FIG. 7—Reversion of framework regions to germline encoded V-gene and J-gene sequences. 4 reversions were made in both the VH and VL in order to generate ATL4894. A NS deamidation site was substituted as indicated by the asterisk (*)

FIG. 8—4894 scFv in phagemid vector for scFV display. Plasmid map of representative antibody scFv cloned into phagemid suitable for expression in bacteria for phage display.

FIG. 9—Outline of cloning and selection steps. Workflow diagram; antibody was subjected to error prone PCR before cloning into phagemid through Gibson cloning (HiFi assembly) and restriction cloning. The phagemid was subjected to three rounds (R1-3) of selections with decreasing ligand concentration to add selection pressure. R1: 1 μg/ml CFH; R2: 0.25 ug/ml CFH, optionally with 1 ug/ml CFHR5 deselection; R3: 0.2 ug/ml CFH, optionally with 1 ug/ml CFHR5 deselection.

FIG. 10—Phage ELISA confirming successful selections. Colonies were picked from round 3 output (Hifi assembly) and phages were purified. ELISA results for binding to reduced CFH (Blue), reduced CFHR5 (red) and lysozyme (green) from phages purified from round 3 selection outputs (HiFi assembly) with CFHR5 deselection (A) and without CFHR5 deselection (B).

FIG. 11—Outline of additional heat challenge and off-rate selection steps. (A) Hifi assembled library was challenged with 3 rounds of selection with decreasing CFH ligand concentration (1, 0.25, and 0.2 ug/ml) with or without 1 μg/ml CFHR5 deselection in R2 and R3 (as outlined in FIG. 10) prior to heat challenge or off rate selection, resulting in four pools. (B) Restriction digest library was challenged with 2 rounds of selection with decreasing CFH ligand concentration (1, and 0.25) with or without 1 μg/ml CFHR5 deselection in R2 prior to heat challenge or off rate selection, resulting in four pools AS IN FIG. 11A.

FIG. 12—Off-rate ELISA. Off-rate ELISA was performed on plates coated with 0.5 ug/ml reduced CFH (Ser860-1231) and 20 ug/ml (a 10-fold molar excess) of 4894 lgG over CFH antigen coated for the off-rate step. In addition, a standard ELISA was performed on lysozyme (1 ug/ml) to detect ‘sticky’ clones (indicated by green dots). Off-rate ELISA were carried out on (A-B) selection outputs from libraries from homology cloning (R HiFi Assembly) or (C-D) selection output from libraries from restriction digests (R2 Restriction digest lib), with and without CFHR5 deselection.

FIG. 13—Results for heat challenge ELISAs. Samples from indicated selection rounds were incubated at 60° C. for 8 minutes followed by a phage ELISA. Absorbance was measured at 450 nm. The 4894 phage is indicated with an asterisk.

FIG. 14—Summary of mutations identified in sequences of ELISA-positive clones from various selection arms. CDR positions (IMGT definition) are shown in red and framework positions are shown in black.

FIG. 15—Results of thermal stability assay by protein denaturation of top 10 variants. (A) Melting curves for top 10 variants and ATL4715, ATL4717, and ATL4894. ATL4830 is an isotype control mAb. (B) Table summarising results with the most stable variants listed at the top.

FIG. 16—Results for heat challenge ELISAs. Top 10 mAbs were incubated at 70° C. for one hour followed by ELISA for binding to reduced CFH.

FIG. 17—THP-1 IL-8 release assay. (A-B) THP-1 cells were incubated with the indicated concentrations of lead mAbs or control antibodies (antibodies irrelevant to antigen) plus LPS. To determine the antibody-dependent IL-8 response, the concentration of IL-8 produced by LPS alone was subtracted from the antibody and LPS IL-8 values.

FIG. 18—ELISA of lead mAbs for binding to CFH protein family members. A. reduced CFH (top) and reduced CFHR3 (bottom). B. reduced CFHR1 (top) and reduced CFHR4 (bottom). C. reduced CFHR2 (top) and reduced CFHR5 (bottom).

FIG. 19—Table showing CDR changes, melting temperatures, heat stability, activity in THP-1 assay and binding to CFH family members in top 10 variants.

FIG. 20—Effects of ATL4717 and ATL5170 on monocyte differentiation towards an activated macrophage-like phenotype. Isolated human monocytes were cultured with ATL4717, ATL5170 and ATL5170Fcnull (Fc domain has no FcR binding capacity). Control samples were incubated with media alone, or an Isotype IgG control antibody. (A) Representative flow cytometry plots showing staining for CD14 and CD11b within CD45+ cells. (B-C) Quantification of CD14-CD11b+ cells (B) and CD14+CD11b+ cells (C).

FIG. 21—Production of inflammatory cytokines in macrophages. Human monocyte-derived macrophages obtained from three donors were incubated with ATL5170 or ATL4892 at a concentration of 0 μg/ml, 1 μg/ml, 10 μg/ml, or 100 μg/ml and the production of IL-6 (A), TNF-alpha (B) and IL-1beta (C) measured.

FIG. 22—Phagocytosis of pHRodo labelled bacteria. Human monocyte-derived macrophages were incubated with ATL5170, ATL4717 in the presence or absence of CFH, and their ability to phagocytose pHRodo labelled bacteria was assessed.

FIG. 23—Effects of ATL5170 on isolated CD4+ T cells. (A) Flow plot showing a change in FSC and SSC, indicative of activation of CD4 T cells as shown by shape change (FSC/SSC by flow cytometry). (B) Quantification of cell trace dye dilution as a measure of CD4+ T cell proliferation. (C) Results of IFN-γ release assay after antibody incubation (ATL5170, anti-PDL1 antibody or combination) without additional activation in CD4+ cells.

FIG. 24—In vitro anti-tumour response of PBMCs treated with ATL5170. PBMCs (with sub-optimal activation by a low concentration IL-2/anti-CD3) were co-cultured with cells from the PDAC10.2 tumour cell line and different concentrations of ATL5170 (2.5 nM, 25 nM, 250 nM) were added to the culture. Graphs show frequency (A) and gMFI (B) of CD69 expression in CD8+ T cells, (C) CD11b+myeloid cells, and (D) CD56+NK cells.

FIG. 25—Dose dependent tumour growth inhibition by ATL5170 in a EMT6-BALB/c syngeneic mouse tumour model. BALB/c mice were inoculated with 5×10\hat{ }6 tumour cells. When MTV reached approximately 70-100 m3, IP dosing at 10 mg/kg with ATL5170 or PBS was initiated. Each mouse received 3 doses in total, 48 hours apart, beginning on day 1. Shown are the mean and standard error of the mean for each treatment group (10 animals/group). IP=intraperitoneally; kg=kilogram; mg=milligram; MTV mean tumour volume

FIG. 26—Dose-dependent PK of ATL5170. (A) Affinity analysis of ATL5170 for binding to immobilised biotinylated full-length CFH (reduced). (B) To measure the pharmacokinetics of ATL5170 in reference to a wild-type human IgG1 (ATL4892), ATL5170 was injected IV once into one of five groups of 12 C57BI/6 mice, at five different doses (0.1 mg/kg, 1 mg/kg, 3 mg/kg, 10 mg/kg, or 20 mg/kg, into healthy C57BI/6 mice. Control mice were injected once with ATL4892 at 10 mg/kg. Blood samples were taken from 8 time points up to 144 hours from initial injection and analysed for detection of ATL5170 or ATL4892. ATL5170 was detected by ELISA at all timepoints equivalent to the control antibody ATL4892, in a dose dependent manner. IgG1=Human Immunoglobulin G1; IV=intravenous; mg=milligram; kg=kilogram; ELISA=enzyme-linked immunosorbent assay; ng=nanogram; hr=hour

FIG. 27—Heatmap showing levels (high/medium/low) of mutational load, CFH mRNA expression, myeloid population, and tumour growth inhibition for selected tumour models (top to bottom): Breast (EMT6), Renal (ENCA), Pancreatic (Pan02), Melanoma (B16F10), Prostate (RM-1), Lymphoma (A20), Melanoma (B16BL6), Colon (CT26), Lung (LL2). ATL5170 had a high level of tumour grown inhibition in breast, renal and pancreatic cancer, and to lesser extent melanomal B16F10.

FIG. 28—Anti-CFH ATL5170 causes an increase in proapoptotic signalling. (A) Histological imaging of tumour tissue sections. C57BI/6 mice were inoculated with EMT6 tumor cells by subcutaneous deposition. When tumors reached 60-100 mm3 in volume, mice were randomized, and selected cohort of mice received treatment with ATL5170 (top row) or vehicle control (bottom row). Shown are ImageXpress PICO imaging system images of tumours following TUNEL (Abcam) and DAPI staining. Mice which had undergone ATL5170 have more TUNEL staining, indicating increased DNA damage and associated induced cancer cell death, relative to vehicle controls. (B) Quantitative analysis of histological imaging. The ATL_5170 treated cohort (left) exhibited statistically higher TUNEL positive cells than the vehicle control treatment group (right); n=3 for all cohorts.

FIG. 29—Serum C3 ELISA of ATL5170 and vehicle treated subjects in a C57BI/6 mouse EMT6 tumour model. Levels of systemic C3 were determined by ELISA in serum samples collected post termination. Absorbance (450 nm) was determined and the concentration of C3 interpolated from the standard curve. No statistically significant difference was observed between ATL5170 treated (left column) and vehicle control treated (right column) cohorts, demonstrating that inhibition of CFH with ATL5170 does not cause depletion of systemic C3.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference.

The antibodies and fragments herein are capable of specifically binding to CFH protein, or a fragment or variant thereof.

As used herein, an antibody capable of “specific binding” or “specifically binding” a target is one able to bind through the association of the epitope recognition site with an epitope within the target. It is distinct from non-specific binding, for example Fc-mediated binding, ionic and/or hydrophobic interactions. In other words, an antibody which specifically binds a target recognise and binds to a specific protein structure within it rather than to proteins generally.

As used herein, “complement factor H (CFH)” or “Factor H” relates to a large (155 kilodaltons), soluble glycoprotein involved in the regulation of the alternative pathway of the complement system, ensuring that the complement system is directed towards pathogens or other dangerous material and does not damage host tissue. CFH is a member of the “regulators of complement activation family” and is a complement control protein. It negatively regulates complement activation on self-cells and surfaces by possessing both cofactor activity for the Factor I mediated C3b cleavage, and decay accelerating activity against the alternative pathway C3-convertase, C3bBb. Factor H exerts a protective action on self-cells and self surfaces but not on the surfaces of bacteria or viruses. However, certain viruses and bacteria have evolved to capture CFH as an immune evasion strategy.

The amino acid sequence of human CFH is provided as SEQ ID NO:24, however as used herein “CFH” includes truncations, derivatives and variants thereof, and may refer to any protein with at least 80%, at least 90% or at least 95% sequence identity to SEQ ID NO:24.

In some embodiments, the antibodies are capable of specifically binding CFH comprising or consisting of amino acid sequence SEQ ID NO:24, or a fragment thereof.

In some embodiments, the antibodies are capable of specifically binding a CFH comprising or consisting of a variant amino acid sequence. In some embodiments, the CFH variant amino acid sequence has at least 70%, at least 80%, at least 85%, at least 90%, at least 95%, at least 95%, or at least 99% identity with SEQ ID NO:24.

In some embodiments, the antibodies are capable of specifically binding a CFH fragment comprising 20%, 30%, 40%, 50%, 60%, 70%, 80%, 90%, 95%, 99% or more of the CFH amino acid sequence, or CFH variant amino acid sequence. Alternatively, the fragment of CFH may comprise 100, 200, 300, 400, 500, 600, 700, 800, 900, 1000, 1100 or 1200 contiguous amino acids of the CFH amino acid sequence, or CFH variant amino acid sequence described above.

In some embodiments, the antibodies are capable of binding to the Sushi 19 (SCR19) domain of a CFH protein. This domain is found at residues 1107-1165 of the full CFH protein, and corresponds to SEQ ID NO: 24. In some embodiments, the SCR19 domain has an amino acid sequence with at least 70%, at least 80%, at least 85%, at least 90%, at least 95%, at least 95%, at least 99%, or 100% identity with SEQ ID NO:24.

In some embodiments, the antibodies bind an epitope within CFH SCR19 domain comprising or consisting of 4-8 contiguous nucleic acids of SEQ ID NO:25. In some embodiments, the antibodies compete, block, or sterically hinder antibodies capable of binding an epitope comprising or consisting of 4-8 contiguous nucleic acids of SEQ ID NO:25, for example in an ELISA assay.

In some embodiments, an antibody capable of binding CFH preferentially binds CFH associated with a cell surface. This may be mediated through a hidden epitope only revealed following conformational changes that result from cell surface association. In some embodiments, the antibody is capable of specific binding to CFH associated with a cell surface with a higher affinity than for free circulating CFH. In some embodiments, the antibody is capable of specific binding to CFH when associated with a cell surface but does not bind free circulating CFH. In some embodiments, the antibody binds to cell surface associated CFH and results in the disassociation of the CFH from the cell surface.

In some embodiments, an antibody capable of binding CFH is also capable of specifically binding one or more other complement related proteins. In particular, an antibody may exhibit specific binding for one or more CFH family protein selected from CFHR1 and/or CFHR2. In some embodiments, CFHR1 and/or CFHR2 are the reduced form.

In some embodiments, an antibody capable of binding CFH is substantially incapable of binding one or more other complement related proteins. In particular, an antibody may be incapable of specific binding to one or more CFH family protein, selected from CFHR3, CFHR4 or CFHR5. In all cases, all CFHR family members are at much lower concentrations than CFH in circulation, and have not been described to be upregulated in tumours. In some embodiments, the antibody is incapable of binding CFHR5. When CFHR5 dimerises it is capable of inducing inflammation via activation of C3b, particularly at the kidney glomerulus Kadhodayi-Kholghi et al 2020. Antibodies increasing cross-linking of CFHR5 may therefore have unwanted inflammatory effects at the kidney glomerulus. Therefore, it may be preferred for the antibodies of the invention not to bind CFHR5. CFHR1 and CFHR2 also have dimerisation domains (de Jorge et al 2013), but the link between dimerisation of these proteins and specific inflammatory consequences is less well understood (de Jorge 2013).

In some embodiments, the antibody is capable of increasing monocyte activation state. In some embodiments, this is independent of FcR engagement. Activation may be determined by increase in CD11b+ and decrease in CD14+ monocytes following contact with the antibody. In some embodiments, antibody is capable of increasing inflammatory cytokine release (e.g. IL-6, TNFa, IL-1b). Without wishing to be bound by theory, this may the result of C3d deposition, and/or due to alterations in CFH interactions with complement receptors on myeloid cells.

In some embodiments, the antibody is capable of increasing complement protein 3b (C3b) and/or C3d deposition on a cell relative to an untreated cell, and/or inhibiting CFH binding to C3b and/or C3d. C3b is the larger of two components formed by cleavage of complement component 3 (C3) by C3 convertase, resulting in the formation of C5 convertase. This in turn cleaves C5 protein which recruits inflammatory cells, initiates the terminal phase of the complement system, and leads to the assembly of the membrane attack complex. C3d is a further cleavage product of C3b, mediated by Factor I, which plays a role in enhancing B cell responses in the classical and lectin pathways. C3b and/or d deposition on a cell is therefore a key step in the activation of both the classical and alternative complement pathway. Inhibiting CFH binding to C3b and/or increasing C3b/C3d deposition on the cell surface results in the activation of the complement system. In some embodiments, C3b/d deposition on a cell is increased by 10%, 20%, 30%, 40%, 50%, 60%, 70%, 80%, 90%, 100% or more relative to an untreated cell.

Antibodies according to the present invention may be provided in isolated form.

By “antibody” we include a fragment or derivative thereof, or a synthetic antibody or synthetic antibody fragment.

In view of today's techniques in relation to monoclonal antibody technology, antibodies can be prepared to most antigens. The antigen-binding portion may be a part of an antibody (for example a Fab fragment) or a synthetic antibody fragment (for example a single chain Fv fragment [ScFv]). Suitable monoclonal antibodies to selected antigens may be prepared by known techniques, for example those disclosed in “Monoclonal Antibodies: A manual of techniques “, H Zola (CRC Press, 1988) and in “Monoclonal Hybridoma Antibodies: Techniques and Applications “, J G R Hurrell (CRC Press, 1982). Chimaeric antibodies are discussed by Neuberger et al (1988, 8th International Biotechnology Symposium Part 2, 792-799).

Monoclonal antibodies (mAbs) are useful in the methods of the invention and are a homogenous population of antibodies specifically targeting a single epitope on an antigen.

Fragments of antibodies, such as Fab and Fab2 fragments may also be provided as can genetically engineered antibodies and antibody fragments. The variable heavy (V_{H}) and variable light (V_{L}) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sd. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the V_{H} and V_{L} partner domains are linked via a flexible oligopeptide (Bird et al (1988) Science 242, 423; Huston et al (1988) Proc. Natl. Acad. Sd. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

By “ScFv molecules” we mean molecules wherein the V_{H} and V_{L} partner domains are covalently linked, e.g. by a flexible oligopeptide.

Fab, Fv, ScFv and dAb antibody fragments can all be expressed in and secreted from E. coli, thus allowing the facile production of large amounts of the said fragments.

Whole antibodies, and F(ab′)2 fragments are “bivalent”. By “bivalent” we mean that the said antibodies and F(ab′)₂ fragments have two antigen combining sites. In contrast, Fab, Fv, ScFv and dAb fragments are monovalent, having only one antigen combining site. Synthetic antibodies which bind to CFH may also be made using phage display technology as is well known in the art.

Antibodies according to the present invention may be detectably labelled or, at least, capable of detection. For example, the antibody may be labelled with a radioactive atom or a coloured molecule or a fluorescent molecule or a molecule which can be readily detected in any other way. Suitable detectable molecules include fluorescent proteins, luciferase, enzyme substrates, and radiolabels. The binding moiety may be directly labelled with a detectable label or it may be indirectly labelled. For example, the binding moiety may be an unlabelled antibody which can be detected by another antibody which is itself labelled. Alternatively, the second antibody may have bound to it biotin and binding of labelled streptavidin to the biotin is used to indirectly label the first antibody.

A “fragment” of an antibody may comprise any number of residues of a “parental” antibody, whilst retaining target binding ability. A fragment may lack effector function, for example may be entirely unable to bind or show diminished binding relative to the parent to the Fc receptor. A fragment is typically smaller than the parental antibody. A fragment may comprise 50%, 60%, 70%, 80%, 90%, 95% or more of the contiguous or non-contiguous amino acids of the parental antibody. A fragment may comprise 50, 100, 150, 200, 250, 300 or more contiguous or non-contiguous amino acids of the parental antibody. A fragment may comprise deletions in the Fc region, or of the Fc region. A fragment may retain the CDRs and/or the variable domains of the parental antibody, unaltered. In some embodiments, a fragment is an Fab fragment or an F(ab′)2 fragment.

Antibodies according to the present invention may comprise the CDRs of antibody ATL5170, i.e.:
i. HCDR1 comprising amino acid sequence SEQ ID NO:12,
ii. HCDR2 comprising amino acid sequence SEQ ID NO:13
iii. HCDR3 comprising amino acid sequence SEQ ID NO:17
iv. LCDR1 comprising amino acid sequence SEQ ID NO:14,
v. LCDR2 comprising amino acid sequence SEQ ID NO:5, and
vi. LCDR3 comprising amino acid sequence SEQ ID NO:6.

The amino acid sequence (and encoding polynucleotide sequence) of the V_{H} and V_{L} regions of ATL5170 have been determined as shown in SEQ ID NOs. 18 and 19 respectively.

Alternatively, antibodies according to the present invention may comprise the CDRs of antibody ATL5155, i.e.:
i. HCDR1 comprising amino acid sequence SEQ ID NO:12,
ii. HCDR2 comprising amino acid sequence SEQ ID NO:13
iii. HCDR3 comprising amino acid sequence SEQ ID NO:3
iv. LCDR1 comprising amino acid sequence SEQ ID NO:14,
v. LCDR2 comprising amino acid sequence SEQ ID NO:5, and
vi. LCDR3 comprising amino acid sequence SEQ ID NO:6.

The amino acid sequence (and encoding polynucleotide sequence) of the V_{H} and V_{L} regions of ATL5155 have been determined as shown in SEQ ID NOs. 15 and 16 respectively.

An antibody may alternatively possess the CDRs of antibody ATL4894, i.e.:
i. HCDR1 comprising amino acid sequence SEQ ID NO:1,
ii. HCDR2 comprising amino acid sequence SEQ ID NO:2
iii. HCDR3 comprising amino acid sequence SEQ ID NO:3
iv. LCDR1 comprising amino acid sequence SEQ ID NO:9,
v. LCDR2 comprising amino acid sequence SEQ ID NO:5, and
vi. LCDR3 comprising amino acid sequence SEQ ID NO:6.

The amino acid sequence (and encoding polynucleotide sequence) of the V_{H} and V_{L} regions of ATL4894 have been determined as shown in SEQ ID NOs. 10 and 11 respectively.

An antibody may alternatively possess the CDRs of antibody ATL4717, i.e.:
i. HCDR1 comprising amino acid sequence SEQ ID NO:1,
ii. HCDR2 comprising amino acid sequence SEQ ID NO:2
iii. HCDR3 comprising amino acid sequence SEQ ID NO:3
iv. LCDR1 comprising amino acid sequence SEQ ID NO:4,
v. LCDR2 comprising amino acid sequence SEQ ID NO:5, and
vi. LCDR3 comprising amino acid sequence SEQ ID NO:6.

The amino acid sequence (and encoding polynucleotide sequence) of the V_{H} and V_{L} regions of ATL4717 have been determined as shown in SEQ ID NOs. 7 and 8 respectively.

In an antibody according to the present invention one or two or three or four of the sequences (i) to (vi) may vary. A variant may have one or two amino acid substitutions in one or two of the sequences (i) to (vi).

The light and heavy chain CDRs 1-3 of ATL5170 or ATL5155 may also be particularly useful in conjunction with a number of different framework regions. Accordingly, light and/or heavy chains having CDRs 1-3 of ATL5170 or ATL5155 may possess an alternative framework region. Suitable framework regions are well known in the art and are described for example in M. Lefranc & G. Le Franc (2001) “The Immunoglobulin Facts Book”, Academic Press, incorporated herein by reference.

In this specification, antibodies may have V_{H} and/or VL regions comprising an amino acid sequence that has a high percentage sequence identity to the ATL5170 of ATL5155 V_{H} and/or V_{L} amino acid sequences.

For example, antibodies according to the present invention include antibodies that bind CFH and have a V_{H} region that comprises an amino acid sequence having at least 70%, more preferably one of at least 75%, 80%, 85%, 90%, 95%, 96%, 97%, 98%, 99% or 100%, sequence identity to the V_{H} region amino acid sequence of ATL5170 (SEQ ID NO: 18) or ATL5155 (SEQ ID NO:15). Alternatively or additionally, the antibodies may have a V_{L} region that comprises an amino acid sequence having at least 70%, more preferably one of at least 75%, 80%, 85%, 90%, 95%, 96%, 97%, 98%, 99% or 100%, sequence identity to the V_{L} region amino acid sequence of ATL5170 (SEQ ID NO:19) or ATL5155 (SEQ ID NO:16).

In some embodiments, antibodies according to the present invention include antibodies that bind CFH and have a heavy chain that comprises an amino acid sequence having at least 70%, more preferably one of at least 75%, 80%, 85%, 90%, 95%, 96%, 97%, 98%, 99% or 100%, sequence identity to the heavy chain amino acid sequence of ATL5170 (SEQ ID NO:22) or ATL5155 (SEQ ID NO:20). Alternatively or additionally, the antibodies may have a light chain that comprises an amino acid sequence having at least 70%, more preferably one of at least 75%, 80%, 85%, 90%, 95%, 96%, 97%, 98%, 99% or 100%, sequence identity to the light chain amino acid sequence of ATL5170 (SEQ ID NO: 23) or ATL5155 (SEQ ID NO:21).

Overall percentage identity of a variable region or full length heavy/light chain sequence may be combined with specified CDR sequence from the same antibody. Therefore, an antibody may comprise a heavy chain comprising CDRH1-3 of ATL5170 within a V_{H} region that comprises an amino acid sequence having at least 70% sequence identity to the V_{H} region amino acid sequence of ATL5170 as outlined above. Similarly, an antibody may comprise a light chain comprising CDLH1-3 of ATL5170 within a V_{L} region that comprises an amino acid sequence having at least 70% sequence identity to the V_{L} region amino acid sequence of ATL5170 as outlined above. An antibody with a heavy chain and/or a light chain with 70% sequence identity to the corresponding chain in ATL5170 may possess the corresponding exact CDR sequences.

Also included are antibodies which “mix and match” the heavy chains from one exemplary antibody with the light chain of another. For example, an antibody having the CDRH1-3, the V_{H} region amino acid sequence of ATL5170 in combination with CDRL1-3, the VL region, or the light chain amino acid sequence amino acid sequence of ATL5155, an antibody having the CDRH1-3, the V_{H} region, or the heavy chain amino acid sequence amino acid sequence of ATL5170 in combination with CDRL1-3, the VL region, or the light chain amino acid sequence amino acid sequence of ATL4894, an antibody having the CDRH1-3, the VH region, or the heavy chain amino acid sequence amino acid sequence of ATL5170 in combination with CDRL1-3, the VL region, or the light chain amino acid sequence amino acid sequence of ATL4717, an antibody having the CDRH1-3, the VH region, or the heavy chain amino acid sequence amino acid sequence of ATL5155 in combination with the V_{L} region amino acid sequence of ATL5170, an antibody having the CDRH1-3, the VH region, or the heavy chain amino acid sequence amino acid sequence of ATL5155 in combination with CDRL1-3, the VL region, or the light chain amino acid sequence amino acid sequence of ATL4894, an antibody having the CDRH1-3, the VH region, or the heavy chain amino acid sequence amino acid sequence of ATL5155 in combination with CDRL1-3, the VL region, or the light chain amino acid sequence amino acid sequence of ATL4717, an antibody having the CDRH1-3, the VH region, or the heavy chain amino acid sequence amino acid sequence of ATL4894 in combination with CDRL1-3, the VL region, or the light chain amino acid sequence amino acid sequence of ATL5170, an antibody having the CDRH1-3, the VH region, or the heavy chain amino acid sequence amino acid sequence of ATL4894 in combination with CDRL1-3, the VL region, or the light chain amino acid sequence amino acid sequence of ATL5155, an antibody having the V_{H} region amino acid sequence of ATL4894 in combination with CDRL1-3, the VL region, or the light chain amino acid sequence amino acid sequence of ATL4717, an antibody having the CDRH1-3, the VH region, or the heavy chain amino acid sequence amino acid sequence of ATL4717 in combination with CDRL1-3, the VL region, or the light chain amino acid sequence amino acid sequence of ATL5170, an antibody having the CDRH1-3, the VH region, or the heavy chain amino acid sequence amino acid sequence of ATL4717 in combination with CDRL1-3, the VL region, or the light chain amino acid sequence amino acid sequence of ATL5155, or an antibody having the V_{H} region amino acid sequence of ATL4717 in combination with CDRL1-3, the VL region, or the light chain amino acid sequence amino acid sequence of ATL4894.

The antibodies of the present invention may possess one or more substitutions within the framework of the V_{H} and/or V_{L} region. As used herein, a “substitution” refers to the exchange of one amino acid for another at a specific position, relative to the same position in a baseline molecule. In some embodiments, the baseline molecules are germline antibodies. In other embodiments, the baseline molecules are the exemplified antibodies provided herein, for example ATL5170 and/or ATL5155.

In some embodiments, the framework substitutions are selected from positions 50, 70 and 123 of the VH domain, and/or 11 and 68 of the VH domain, according to IMGT numbering (Lefranc, M.-P., Immunology Today, 18, 509 (1997) PMID: 9386342). In some embodiments, the framework substitutions are at positions selected from VH domain substitutions L50, S70, and L123, and/or VL substitutions L11 and E68. In some embodiments the substitutions are selected from VH domain substitutions L50P, S70G, and L123Q, and/or VL substitutions L11Q and E68v. In some embodiments, the antibody comprises 1, 2, 3, 4, 5, or more framework substitutions within the V_{H} and V_{L} region combined. In some embodiments, the antibody comprises no more than 1, 2, 3, 4, or 5 framework substitutions within the V_{H} and V_{L} region combined.

In some embodiments, antibodies possess one or more of the following residues at the following positions: V_{H} domain 50P, 70G, and 123Q, and/or V_{L} domain 11Q and 68V, according to IMGT numbering. In some embodiments, the antibody comprises 1, 2, 3, 4, 5, of these residues at the specified positions.

In some embodiments, the antibodies according to the present invention include antibodies that bind CFH and have a V_{H} region that comprises the V_{H} region amino acid sequence of ATL5170 (SEQ ID NO:18) or ATL5155 (SEQ ID NO:15) with one or more substitutions selected from L50P, S70G, and L123Q (IMGT numbering). Alternatively, or additionally, the antibodies may have a V_{L} region that comprises the V_{L} region amino acid sequence of ATL5170 (SEQ ID NO:19) or ATL5155 (SEQ ID NO:16) with one or more substitutions selected from L11Q and E68V.

For example:
an antibody comprising a V_{H} region that comprises the V_{H} region amino acid sequence of ATL5170 (SEQ ID NO:18) with the substitutions L50P, S70G, and L123Q (IMGT numbering) and a V_{L} region that comprises the V_{L} region amino acid sequence of ATL5170 (SEQ ID NO:19)·
an antibody comprising a V_{H} region that comprises the V_{H} region amino acid sequence of ATL5170 (SEQ ID NO:18) with the substitutions L50P, S70G, and L123Q (IMGT numbering) and a V_{L} region that comprises the V_{L} region amino acid sequence of ATL5170 (SEQ ID NO: 19) with the substitution L11Q.
an antibody comprising a V_{H} region that comprises the V_{H} region amino acid sequence of ATL5170 (SEQ ID NO:18) with the substitutions L50P, S70G, and L123Q (IMGT numbering) and a V_{L} region that comprises the V_{L} region amino acid sequence of ATL5170 (SEQ ID NO: 19) with the substitution E68V.
an antibody comprising a V_{H} region that comprises the V_{H} region amino acid sequence of ATL5170 (SEQ ID NO: 18) with the substitutions L50P, S70G, and L123Q (IMGT numbering) and a V_{L} region that comprises the V_{L} region amino acid sequence of ATL5170 (SEQ ID NO: 19) with substitutions at L11Q and E68V.
an antibody comprising a V_{H} region that comprises the V_{H} region amino acid sequence of ATL5155 (SEQ ID NO: 15) with the substitutions L50P, S70G, and L123Q (IMGT numbering) and a V_{L} region that comprises the V_{L} region amino acid sequence of ATL5155 (SEQ ID NO:16)
an antibody comprising a V_{H} region that comprises the V_{H} region amino acid sequence of ATL5155 (SEQ ID NO:15) with the substitutions L50P, S70G, and L123Q (IMGT numbering) and a V_{L} region that comprises the V_{L} region amino acid sequence of ATL5155 (SEQ ID NO:16) with the substitution L11Q.
an antibody comprising a V_{H} region that comprises the V_{H} region amino acid sequence of ATL5155 (SEQ ID NO: 15) with the substitutions L50P, S70G, and L123Q (IMGT numbering) and a V_{L} region that comprises the V_{L} region amino acid sequence of ATL5155 (SEQ ID NO:16) with the substitution E68V.
an antibody comprising a V_{H} region that comprises the V_{H} region amino acid sequence of ATL5155 (SEQ ID NO:15) with the substitutions L50P, S70G, and L123Q (IMGT numbering) and a V_{L} region that comprises the V_{L} region amino acid sequence of ATL5155 (SEQ ID NO: 16) with substitutions at L11Q and E68V.

Sequences and properties of antibodies of the invention may be compared to a “reference antibody”. As used herein, a “reference antibody” is an antibody which binds the same target as the antibodies of the invention, but differs in one or more physical property. For example, a reference antibody may differ in at least one amino acid residue in CDRH1, CDRH2, CDRH3, CDRL1, CDRL2, CDRL3, the VH framework, the VL framework, the heavy chain backbone, the light chain backbone, the Fc region and/or the hinge region, so long as they bind to the same target, preferably the same epitope, as the antibodies of the invention. Reference antibodies may be isotype matched to the antibodies of the invention. Reference antibodies may bind the same epitope, or block, sterically hinder or otherwise compete for the same epitope as the antibodies of the invention. Reference antibodies may be known in the art, or may possess the CDRs and/or variable domains of an antibody in the art whilst being otherwise identical to the antibodies of the invention. For example, ATL4715 is an exemplary reference antibody which possesses VH and VL domains (SEQ ID NO: 27 and 28) previously disclosed in PCT/US2014/041441, within an IgG1 backbone. Another exemplary reference antibody is ATL4717, obtainable as described in Example 1.

Preferred antibodies possess one or more residue that differs from a reference antibody capable of binding the same target (for example ATL4717 and/or ATL4715), and have one or more improved property relative to said reference antibody. Differences may be in the CDRs and/or framework residues of the variable domains. In some embodiments, the antibodies differ from a reference antibody in their CDRs, and can bind the same target, optionally at the same or a similar epitope. For example, a substituted antibody may exhibit improved thermostability, half-life, shelf-life, or tolerability. It is non-trivial to identify what, if any, residues within an antibody may improve one or more properties, without the exercise of hindsight. This may be achieved by taking a “parent” reference antibody and painstakingly altering individual, or combinations of, residues and analyzing the results. Alternatively, guided design such as analysis of variants within naturally arising antibody families so as to identify candidate substitutions, or phage display analysis of antibodies produced by error-prone PCR, may be performed and collated to produce improved antibodies. Candidate substitutions may be identified from multiple sources and combined for further testing to produce even more advantageous antibodies. For example, it was only through the significant investigation and guided design described herein that the present inventors were able to produce antibodies ATL5155 and ATL5170 which exhibit enhanced properties over parental antibody ATL4717.

In some embodiments, the antibodies possess enhanced heat stability. Enhanced heat stability may be the result of one or more residues in the CDRs, or of framework residues within the V_{H} or V_{L} regions. Antibody stability is a complex trait which greatly influences performance (i.e. specificity, tolerability, and affinity). Poor stability can lead to antibody misfolding, resulting in a low product yield and a substantial fraction of inactive material, as well as degradation during storage.

Heat stability may be quantified using any technique known in the art. A variety of assays are available, including differential scanning calorimetry (Vermeer and Norde 2000; Vermeer et al. 2000; Garber and Demarest 2007; Brader et al. 2015), circular dichroism, and thermal shift assays, for example differential scanning fluorimetry (DSC) assays e.g. those employing SYPRO™ Orange (Bio Rad, US). In DSC assays, an antibody is gradually heated in the presence of a hydrophobic fluorescent dye. As the antibody unfolds, the dye binds to newly exposed hydrophobic patches. The temperature where the fluorescence increases the most rapidly is termed a thermal unfolding transitions or melting temperature (Tₘ). Antibodies possess multiple distinct melting temperatures: Tₘ1 is the lowest temperature, and represents the unfolding of the C_{H}2 domain, with Tₘ2 and Tₘ3 correspond to the melting of the C_{H}3 and FAB domains, and occurring at higher temperatures. Depending on pH and buffer conditions, Tₘ1 and Tₘ2 may occur simultaneously, as may Tₘ2 and Tₘ3, to produce a two-phase melt curve. Following Tₘ3, the antibody is fully denatured and no further melting can occur. An exemplary assay is outlined in Example 3.

In some embodiments, the antibodies of the invention having improved heat stability have a higher Tₘ1, Tₘ2 and/or Tₘ3 value than a reference antibody. In some embodiments, the antibodies of the invention having improved heat stability have a Tₘ3 value (i.e. is completely melted) of 78° C. or more, 79° C. or more, 80° C. or more, 81° C. or more, 82° C. or more, 83° C. or more, or 84° C. or more. In some embodiments, the antibodies of the invention having improved heat stability have a Tₘ3 value of 84+2° C., 84+3° C., 84+3° C., 84+4° C., or 84+5° C.

Thermal stability may also be measured through heat challenge ELISA, whereby antibodies are exposed to high heat (for example 70° C.) for a prolonged period (e.g. 1 hour) prior to ELISA against their target protein or an epitope-containing fragment thereof. An exemplary assay is outlined in Example 3.

Enhanced heat stability may be expressed as relative to a reference antibody under similar, preferably identical, conditions. In some embodiments, a reference antibody differs from the antibodies of the invention in one or more residue in the CDRs or in the V_{H} or V_{L} regions. Preferably, the reference antibody binds the same target epitope as the antibody of the invention. A reference antibody may differ from the antibody of the invention only in the V_{H} and/or V_{L} regions, in one or more CDRs, and/or in one or more residues within the CDRs or framework residues within the V_{H} or V_{L} regions. In other embodiments, enhanced heat stability is relative to a reference antibody selected from ATL4717, ATL4715, an antibody possessing the VH and VL regions of said reference antibody, or an isotype thereof.

In some embodiments, antibodies of the invention having improved heat stability have a Tₘ3 value which is at least 1° C., at least 2° C., at least 3° C., at least 4° C., or at least 5° C. higher than a reference antibody. In some embodiments, antibodies of the invention having improved heat stability outperform a reference antibody in a heat challenge ELISA assay.

In some embodiments, the isolated antibody or antibody fragment may comprise a heavy chain immunoglobulin constant domain selected from the group consisting of a human IgM constant domain, a human IgG4 constant domain, a human IgG1 constant domain, a human IgE constant domain, a human IgG2 constant domain, a human IgG3 constant domain, and a human IgA constant domain. In some embodiments, the isolated antibody or antibody fragment is not an autoantibody.

Whilst any antibody isotype may be used in the present invention, in some embodiments the antibody is an lgG isotype (e.g. selected from IgG1-4). In some embodiments, the antibody is an lgG subclass selected from IgG1 and IgG3. In some embodiments, the antibody is IgG3. In preferred embodiments, the antibody is IgG1. Advantageously, IgG1 antibodies combine high stability, reduced aggregation and fragmentation, good tolerability, a long circulating half-life in vivo, improved or favourable pharmacokinetics and ease of manufacture. Despite IgG3's association with complement activation, the advantageous properties of IgG1 make it surprisingly suitable choice for antibodies targeting CFH.

Comparative qualities (for example heat stability) are preferably made in reference to an antibody having the same isotype as the antibodies of the invention.

Isolated nucleic acids encoding an antibody, antigen binding fragment, or polypeptide as described herein are provided.

The nucleic acid may encode an amino acid sequence of one of SEQ ID NOs 15, 16, or 18-23, or may encode an amino acid sequence having at least 70% identity thereto, optionally one of 75%, 80%, 85% or one of 86%, 87%, 88%, 89%, 90%, 91%, 92%, 93%, 94%, 95%, 96%, 97%, 98%, 99%, or 100% of SEQ ID NOs 15, 16, or 18-23.

The nucleic acid may have a sequence of one of SEQ ID NOs 32-35 or may have nucleotide sequence having at least 70% identity thereto, optionally one of 75%, 80%, 85% or one of 86%, 87%, 88%, 89%, 90%, 91%, 92%, 93%, 94%, 95%, 96%, 97%, 98%, 99%, or 100% of SEQ ID NOs 33-36. In some embodiments, the nucleic acid has a sequence of SEQ ID NOs 32 and 33, or of SEQ ID NO 34 and 35.

Also provided is a vector comprising a nucleic acid described herein, and a host cell comprising the vector. For example, the host cell may be a eukaryotic, or mammalian, e.g. Chinese Hamster Ovary (CHO), cell or may be a prokaryotic cell, e.g. E. coli. In some embodiments, the vector is a viral vector, for example a bacteriophage.

Further provided are methods for making an antibody, or antigen binding fragment or polypeptide as described herein is provided, the method comprising culturing a host cell as described herein under conditions suitable for the expression of a vector encoding the antibody, or antigen binding fragment or polypeptide, and recovering the antibody, or antigen binding fragment or polypeptide.

The antibodies and fragments thereof described herein may find use in therapy. CFH is a regulator of the complement system, an effector for response to many diseases and conditions. Complement leads to numerous outcomes detrimental to invaders and malignancies, including direct killing by formation of the pore-forming membrane attack complex, recruitment of immune cells to sites of invasion, facilitation of phagocytosis, and enhancement of cellular immune responses. CFH association on a cell surface suppresses alternative complement-mediated attack by accelerating decay of convertases and by helping to inactivate C3 fragments, and is key for balancing collateral damage caused by the complement system. However, pathogens and malignancies must overcome the complement system to survive in the host, and a common strategy used by pathogens and malignancies to evade complement is hijacking host complement regulators. CFH is a key target for such “hijacking” (Moore et al, 2021) and, as a consequence, the antibodies as described herein find use in the treatment of a wide range of diseases and conditions, including those caused by pathogens (including bacteria, viruses, protozoa, helminths, and fungi) and neoplasms which use CFH to evade complement attack.

A subject to be treated may be any animal or human. The subject is preferably mammalian, more preferably human. The subject may be male or female. The subject may be a patient. Therapeutic uses may be in human or animals (veterinary use).

Medicaments and pharmaceutical compositions according to aspects of the present invention may be formulated for administration by a number of routes, including but not limited to, parenteral, intravenous, intra-arterial, intramuscular, intratumoural, oral and nasal. The medicaments and compositions may be formulated for injection.

Pharmaceutical compositions may be prepared using a pharmaceutically acceptable “carrier” composed of materials that are considered safe and effective. “Pharmaceutically acceptable” refers to molecular entities and compositions that are “generally regarded as safe”, e.g., that are physiologically tolerable and do not typically produce an allergic or similar untoward reaction, such as gastric upset and the like, when administered to a human. In some embodiments, this term refers to molecular entities and compositions approved by a regulatory agency of the US federal or a state government, as the GRAS list under section 204 (s) and 409 of the Federal Food, Drug and Cosmetic Act, that is subject to premarket review and approval by the FDA or similar lists, the U.S. Pharmacopeia or another generally recognised pharmacopeia for use in animals, and more particularly in humans.

The term “carrier” refers to diluents, binders, lubricants and disintegrants. Those with skill in the art are familiar with such pharmaceutical carriers and methods of compounding pharmaceutical compositions using such carriers.

The pharmaceutical compositions provided herein may include one or more excipients, e.g., solvents, solubility enhancers, suspending agents, buffering agents, isotonicity agents, antioxidants or antimicrobial preservatives. When used, the excipients of the compositions will not adversely affect the stability, bioavailability, safety, and/or efficacy of the active ingredients, i.e. the anti-CFH antibodies used in the composition. Thus, the skilled person will appreciate that compositions are provided wherein there is no incompatibility between any of the components of the dosage form. Excipients may be selected from the group consisting of buffering agents, solubilizing agents, tonicity agents, chelating agents, antioxidants, antimicrobial agents, and preservatives.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc., is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 20th Edition, 2000, pub. Lippincott, Williams & Wilkins.

A “cancer” can comprise any one or more of the following: acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, stomach cancer, testicular cancer, thyroid cancer, urinary system cancer, uterine sarcoma, vaginal cancer, vascular system, Waldenstrom's macroglobulinemia and Wilms' tumor.

In some embodiments, the cancer is selected from breast cancer, renal cancer, pancreatic cancer, or melanoma. In some embodiments, the cancer is selected from breast, renal, or pancreatic cancer.

Cancers may be of a particular type. Examples of types of cancer include astrocytoma, carcinoma (e.g. adenocarcinoma, hepatocellular carcinoma, medullary carcinoma, papillary carcinoma, squamous cell carcinoma), glioma, lymphoma, medulloblastoma, melanoma, myeloma, meningioma, neuroblastoma, sarcoma (e.g. angiosarcoma, chondrosarcoma, osteosarcoma).

The anti-CFH antibodies of the invention are especially contemplated for use in the treatment of immunotherapy resistant cancers. As used herein, a cancer is “immunotherapy resistant” if it has not previously responded, or is expected not to in future respond, to immunotherapy. In some instances, an immunotherapy resistant cancer has already been treated with an immunotherapeutic regimen, and has exhibited a “low responsiveness” or “resistance”, i.e. has not responded to treatment, failed to enter or stalled within remission, or has subsequently recurred following a period of remission. In others, it has been diagnosed as immunotherapy resistant, for example through the presence or absence of one or more biomarker or genetic marker associated with expected low responsiveness to immunotherapy. In particular, immunotherapy resistant cancers may be those which have with low responsiveness or resistance to immune checkpoint inhibitors, and/or complement mediated therapies.

As used herein, an “immune checkpoint inhibitor” includes PD1 and PDL1 inhibitors, CTLA-4 inhibitors, CD40 agonists, macrophage activators (such as Clever-1 inhibition, CD47 inhibition, CSF-R1 activation), complement activators (such as cetuximab), T cell/DC activators (such as OX40, CD137, CXCR4), and NK activators such as KIR inhibitors. The antibodies of the invention, when used in therapy, may be combined with therapy using immune checkpoint inhibitors, in particular when used to treat an immune checkpoint inhibitor resistant cancer.

Suitable cancers for treatment as described herein include cancers expressing high levels of sialic acid motifs or other neoantigens likely to result in increased sequestering of CFH. Particularly suitable are cancers expressing high levels of CFH. For example, high levels of CFH are currently reported in NSCLC, adenocarcinomas, colorectal cancers, breast cancers, ovarian cancers, liver cancers. These cancers are likely to respond especially well to therapies targeting CFH.

Cancers that may particularly respond to treatment as described herein are cancers associated with high, elevated or increased CFH expression, high mutational burden, and/or high immune cell infiltration.

Infectious diseases especially amenable to treatment with the antibodies and fragments thereof as outlined herein include those caused by pathogens (including bacteria, viruses, protozoa, helminths, and fungi) which interact with CFH and/or which use CFH to evade complement attack. Exemplary pathogens include bacteria such as Acinetobacter baumanni, Bacillus anthracis, Borrelia spp. (e.g. B. burgdorferi, B. hermsii, B. mayonii, B. miyamotoi, B. parkeri, B. recurrentis, B. spielmanii), Escherichia coli, Fancisella tularensis, Fusobacterium necrophorum, Haemophilus somni, Leptospira spp., Moraxella catarrhalis, Mycoplasma hypopneumoniae, Neisserie spp. (e.g. N. cinerea, N. gonorrhoeae, N. meningitidis), Haemophilus influenzae (e.g. type b, type f, non-typeable), Pasteurella pneumonotropica, Pseudomonas aeruginosa, Rickettsia conorii, Salmonella spp., Staphylococcus aureus, Streptococcus spp. (e.g. S. agalactiae, S. pneumoniae, S. pyrogenes, S. suis), Treponema denticola, Yersinia spp. (e.g. Y. enterocolitica, and Y. pseudotuberculosis). Also included are fungi (Aspergillus fumigatus), protozoa (Trypanosoma brucei, Trypanosoma cruzi, Toxoplasma gondii, Plasmodium falciparum), helminths (Echinococcus granulosus, Onchocerca volvulus) and viruses (Human Immunodeficiency Virus (HIV), West Nile Virus, and Ebola virus) which interact with CFH. The interaction of these pathogens with CFH is outlined in Moore et al, 2021.

Infectious diseases caused by pathogens which interfere with CFH polyanion binding of binding to C3 fragments, or otherwise interact with SSH domains 18-20 of CFH, may be especially amenable to treatment with the antibodies of the invention, as these bind within SSH domains 18-20 which mediate these effects. Exemplary pathogens include B. hermsii, C. albicans, Leptospria spp, N. gonorrhoeae, N. meningitidis, P. falciparum, P. aeruginosa, Salmonella spp, S. aureus, S. pneumoniae, S. pyogenes, Y. pseudotuberculosis, A. fumigatus, Bordetella spp, B. afzelii, B. burgdorferi, B. miyamotoi, B. parkeri, B. recurrentis, B. spielmanii, E. coli, F. necrophorum, and H. influenzae type b and f.

Subjects may be particularly amenable to treatment described herein if they possess high, elevated or increased levels of one or more biomarkers selected from CFH expression, high mutational burden, and/or high immune cell infiltration. Where the treatment is for treating an infectious disease, the biomarkers may be selected from CFH expression and/or high immune cell infiltration. Whether a subject has “high”, “elevated” or “increased” CFH expression, mutational burden and/or immune cell infiltration may be determined through comparison to a reference. Suitable references may include experimentally determined references, for example by obtaining or providing a reference sample and determining the levels of CFH expression, mutational burden and/or immune cell infiltration within it. Reference samples include tissue samples, preferably a somatic tissue and samples of a reference cancer. Reference cancers are preferably of a corresponding or the same tissue and tumour type to the cancer to be treated (e.g. both the cancer to be treated or queried and the reference cancer are breast cancers). Reference samples may be obtained from the subject, and may be obtained prior (for example, one or more weeks, months, or years previously) to the sample of the cancer to which the reference is compared. Alternatively, a reference may also be a predetermined or predicted value, and may take the form of a standard, look up table, or a threshold value.

In some embodiments, the subject is selected for treatment on the basis of high or elevated CFH expression, mutational burden and/or immune cell infiltration. In some embodiments, the selection comprises obtaining or providing a sample from a subject, for example a tumour sample, and determining the level of CFH expression, mutational burden and/or immune cell infiltration within it. The selection may additionally comprise comparing the levels so determined to a reference level. Suitable references may include experimentally determined references, for example by obtaining or providing a sample of a reference cancer or tissue and determining the levels of CFH expression, mutational burden and/or immune cell infiltration within it. A reference may also be a predetermined or predicted value, and may take the form of a standard, look up table, or a threshold value.

The antibodies of the invention may be used in therapy with further therapeutic agents. As used herein, a “further therapeutic agent” is an additional compound, protein, vector, antibody, cell or entity with a therapeutic effect. For example, where the antibodies are used to treat cancers, the further therapeutic agent may be a chemotherapeutic agent, a radiotherapeutic agent, an immune checkpoint inhibitor, or an antibody with cancer-killing cytotoxic function. Where the antibodies are used to treat infectious disease, further therapeutic agents include further antibodies against the pathogenic cause of the disease, antibiotics, antifungal agents, antiparasitic agents, antiviral agents, and/or anti-helmetics effective against the disease.

The antibodies may be co-administered with a further therapeutic agent. The antibodies may be co-formulated with a further therapeutic agent. The antibodies may be sequentially administered before or after a further therapeutic agent.

The antibodies find particular use for activating the complement system in a subject in need thereof. As used herein, “activating the complement system” may mean increasing complement system mediated killing of cells and/or pathogens through the classic, alternative, or lectin pathways. The subject may have a condition resulting in an exhausted or suppressed complement system, for example a disease or disorder whereby CFH is recruited to the surface of particles of the causative agent of, or to the surface of cells infected or affected by, the disease or disorder. The antibodies may be used to relieve complement exhaustion or suppression in a subject.

Some methods of the present invention involve a sample containing cells. The sample may be a culture of cells grown in vitro. For example, the culture may comprise a suspension of cells or cells cultured in a culture plate or dish.

Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism.

According to some aspects of the present invention a kit of parts is provided comprising an antibody according to the present invention.

In some embodiments, the kit comprises an antibody according to the present invention and one or more of: reagents for use in immunochemistry; the antibodies immobilised to a solid support; means for labelling the antibodies; means for linking the antibodies to a cytotoxic moiety; a further therapeutic agent.

Percentage (%) sequence identity is defined as the percentage of amino acid residues in a candidate sequence that are identical with residues in the given listed sequence (referred to by the SEQ ID NO) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

Alignment for purposes of determining percent amino acid sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

The invention includes the combination of the aspects and preferred features described except where such a combination is clearly impermissible or expressly avoided.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example+/−10%.

### Sequences

TABLE 0
Sequences

| SEQ ID NO: | Description | Sequence |
|---|---|---|
| 1. | ATL4717 CDRH1 aa | GFTFSIYG |
| 2. | ATL4717 CDRH2 aa | MSFDGKTK |
| 3. | ATL4717 CDRH3 aa | AKGGATAAVFDS |
| 4. | ATL4717 CDRL1 aa | QSLLYRSNSKNY |
| 5. | ATL4717 CDRL2 aa | WAS |
| 6. | ATL4717 CDRL3 aa | QQYYNPPWT |
| 7. | ATL4717 VH aa | QVQLVESGGGMVQPGRSLRLSCAASGFTFSIYGMHWVRQA |
| | | PGRGLEWVAVMSFDGKTKYYADSVKGRFTISRDNSKNTLY |
| | | LQMNSLRRVDTAVYYCAKGGATAAVFDSWGQGTLVTVSS |
| 8. | ATL4717 VL aa | DIVMTQSPDSLAVSLGERAAINCKSSQSLLYRSNSKNYLAWY |
| | | QQQPGQPPKLLVYWASTRESGVPDRFSGSGSGTDFTLTISS |
| | | LQAEDVAVYYCQQYYNPPWTFGRGTKVEIK |
| 9. | ATL4894 CDRL1 aa | QSLLYRSNNKNY |
| 10. | ATL4894 VH aa | QVQLVESGGGVVQPGRSLRLSCAASGFTFSIYGMHWVRQA |
| | | PGKGLEWVAVMSFDGKTKYYADSVKGRFTISRDNSKNTLYL |
| | | QMNSLRAEDTAVYYCAKGGATAAVFDSWGQGTLVTVS |
| 11. | ATL4894 VL aa | DIVMTQSPDSLAVSLGERATINCKSSQSLLYRSNNKNYLAWY |
| | | QQKPGQPPKLLIYWASTRESGVPDRFSGSGSGTDFTLTISSL |
| | | QAEDVAVYYCQQYYNPPWTFGQGTKVEIK |
| 12. | ATL5155 CDRH1 aa | GFTFSNYG |
| 13. | ATL5155 CDRH2 aa | TSFDGKTK |
| 14. | ATL5155 CDRL1 aa | QSLLYRRNNKNY |
| 15. | ATL5155 VH aa | QVQLVESGGGVVQPGRSLRLSCAASGFTFSNYGMHWVRQA |
| | | PGKGLEWVAVTSFDGKTKYYADSVKGRFTISRDNSKNTLY |
| | | LQMNSLRAEDTAVYYCAKGGATAAVFDSWGQGTLVTVSS |
| 16. | ATL5155 VL aa | DIVMTQSPDSLAVSLGERATINCKSSQSLLYRRNNKNYLAWY |
| | | QQKPGQPPKLLIYWASTRESGVPDRFSGSGSGTDFTLTISSL |
| | | QAEDVAVYYCQQYYNPPWTFGQGTKVEIK |
| 17. | ATL5170 CDRH3 aa | AKGGATAAVLDS |
| 18. | ATL5170 VH aa | QVQLVESGGGVVQPGRSLRLSCAASGFTFSNYGMHWVRQA |
| | | PGKGLEWVAVTSFDGKTKYYADSVKGRFTISRDNSKNTLY |
| | | LQMNSLRAEDTAVYYCAKGGATAAVLDSWGQGTLVTVSS |
| 19. | ATL5170 VL aa | DIVMTQSPDSLAVSLGERATINCKSSQSLLYRRNNKNYLAWY |
| | | QQKPGQPPKLLIYWASTRESGVPDRFSGSGSGTDFTLTISSL |
| | | QAEDVAVYYCQQYYNPPWTFGQGTKVEIK |
| 20. | ATL5155 Hc full aa | QVQLVESGGGVVQPGRSLRLSCAASGFTFSNYGMHWVRQA |
| | | PGKGLEWVAVTSFDGKTKYYADSVKGRFTISRDNSKNTLY |
| | | LQMNSLRAEDTAVYYCAKGGATAAVFDSWGQGTLVTVSSA |
| | | STKGPSVFPLAPSSKSTSGGTAALGCLVKDYFPEPVTVSW |
| | | NSGALTSGVHTFPAVLQSSGLYSLSSVVTVPSSSLGTQTY |
| | | ICNVNHKPSNTKVDKKVEPKSCDKTHTCPPCPAPELLGGP |
| | | SVFLFPPKPKDTLMISRTPEVTCVVVDVSHEDPEVKFNWY |
| | | VDGVEVHNAKTKPREEQYNSTYRVVSVLTVLHQDWLNGKE |
| | | YKCKVSNKALPAPIEKTISKAKGQPREPQVYTLPPSRDEL |
| | | TKNQVSLTCLVKGFYPSDIAVEWESNGQPENNYKTTPPVL |
| | | DSDGSFFLYSKLTVDKSRWQQGNVFSCSVMHEALHNHYTQ |
| | | KSLSLSPGK |
| 21. | ATL5155 Lc full aa | DIVMTQSPDSLAVSLGERATINCKSSQSLLYRRNNKNYLAWY |
| | | QQKPGQPPKLLIYWASTRESGVPDRFSGSGSGTDFTLTISSL |
| | | QAEDVAVYYCQQYYNPPWTFGQGTKVEIKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQG |
| | | LSSPVTKSFNRGEC |
| 22. | ATL5170 Hc full aa | QVQLVESGGGVVQPGRSLRLSCAASGFTFSNYGMHWVRQA |
| | | PGKGLEWVAVTSFDGKTKYYADSVKGRFTISRDNSKNTLY |
| | | LQMNSLRAEDTAVYYCAKGGATAAVLDSWGQGTLVTVSSA |
| | | STKGPSVFPLAPSSKSTSGGTAALGCLVKDYFPEPVTVSWN |
| | | SGALTSGVHTFPAVLQSSGLYSLSSVVTVPSSSLGTQTYICN |
| | | VNHKPSNTKVDKKVEPKSCDKTHTCPPCPAPELLGGPSVFL |
| | | FPPKPKDTLMISRTPEVTCVVVDVSHEDPEVKFNWYVDGVE |
| | | VHNAKTKPREEQYNSTYRVVSVLTVLHQDWLNGKEYKCKVS |
| | | NKALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| 23. | ATL5170 Lc full aa | DIVMTQSPDSLAVSLGERATINCKSSQSLLYRRNNKNYLAWY |
| | | QQKPGQPPKLLIYWASTRESGVPDRFSGSGSGTDFTLTISSL |
| | | QAEDVAVYYCQQYYNPPWTFGQGTKVEIKRTVAAPSVFIFP |
| | | PSDEQLKSGTASVVCLLNNFYPREAKVQWKVDNALQSGNS |
| | | QESVTEQDSKDSTYSLSSTLTLSKADYEKHKVYACEVTHQG |
| | | LSSPVTKSFNRGEC |
| 24. | Complement Factor | MRLLAKIICLMLWAICVAEDCNELPPRRNTEILTGSWSDQTY |
| | H full aa | PEGTQAIYKCRPGYRSLGNVIMVCRKGEWVALNPLRKCQKRP |
| | | CGHPGDTPFGTFTLTGGNVFEYGVKAVYTCNEGYQLLGEINY |
| | | RECDTDGWTNDIPICEVVKCLPVTAPENGKIVSSAMEPDRE |
| | | YHFGQAVRFVCNSGYKIEGDEEMHCSDDGFWSKEKPKCVEI |
| | | SCKSPDVINGSPISQKIIYKENERFQYKCNMGYEYSERGDAV |
| | | CTESGWRPLPSCEEKSCDNPYIPNGDYSPLRIKHRTGDEITY |
| | | QCRNGFYPATRGNTAKCTSTGWIPAPRCTLKPCDYPDIKHG |
| | | GLYHENMRRPYFPVAVGKYYSYYCDEHFETPSGSYWDHIH |
| | | CTQDGWSPAVPCLRKCYFPYLENGYNQNYGRKFVQGKSID |
| | | VACHPGYALPKAQTTVTCMENGWSPTPRCIRVKTCSKSSIDI |
| | | ENGFISESQYTYALKEKAKYQCKLGYVTADGETSGSITCGKD |
| | | GWSAQPTCIKSCDIPVFMNARTKNDFTWFKLNDTLDYECHD |
| | | GYESNTGSTTGSIVCGYNGWSDLPICYERECELPKIDVHLVP |
| | | DRKKDQYKVGEVLKFSCKPGFTIVGPNSVQCYHFGLSPDLPI |
| | | CKEQVQSCGPPPELLNGNVKEKTKEEYGHSEVVEYYCNPR |
| | | FLMKGPNKIQCVDGEWTTLPVCIVEESTCGDIPELEHGWAQL |
| | | SSPPYYYGDSVEFNCSESFTMIGHRSITCIHGVWTQLPQCVA |
| | | IDKLKKCKSSNLIILEEHLKNKKEFDHNSNIRYRCRGKEGWI |
| | | HTVCINGRWDPEVNCSMAQIQLCPPPPQIPNSHNMTTTLNYR |
| | | DGEKVSVLCQENYLIQEGEEITCKDGRWQSIPLCVEKIPCSQ |
| | | PPQIEHGTINSSRSSQESYAHGTKLSYTCEGGFRISEENETT |
| | | CYMGKWSSPPQCEGLPCKSPPEISHGVVAHMSDSYQYGEEV |
| | | TYKCFEGFGIDGPAIAKCLGEKWSHPPSCIKTDCLSLPSFE |
| | | NAIPMGEKKDVYKAGEQVTYTCATYYKMDGASNVTCINSRW |
| | | TGRPTCRDTSCVNPPTVQNAYIVSRQMSKYPSGERVRYQC |
| | | RSPYEMFGDEEVMCLNGNWTEPPQCKDSTGKCGPPPPIDN |
| | | GDITSFPLSVYAPASSVEYQCQNLYQLEGNKRITCRNGQWS |
| | | EPPKCLHPCVISREIMENYNIALRWTAKQKLYSRTGESVEFV |
| | | CKRGYRLSSRSHTLRTTCWDGKLEYPTCAKR |
| 25. | Complement Factor | GKCGPPPPIDNGDITSFPLSVYAPASSVEYQCQNLYQLEGNK |
| | H SCR19 domain aa | RITCRNGQWSEPPKCLH |
| | (1107-1165) | |
| 26. | Complement Factor | KCGPPPPIDNGDITSFPLSVYAPASSVEYQCQNLYQLEGNKR |
| | H SCR19 domain aa | ITCRNGQWSEPPKCLH |
| | (1108-1165) | |
| 27. | ATL4715 VH | EVQLVESGGGVVQPGRSLRLSCAASGFTFSRYGMHWVRQA |
| | | PGKGLEWVAVMSYDGSTKYYADSVKGRFAISRDNPKNTLFL |
| | | QMNSLRPDDTAVYYCAKGGAAAAVMDVWGKGTTVTVSS |
| 28. | ATL4715 VL | DIVMTQSPDSLTVSLGERATISCKSSQRLLYSSNNKNYLAWY |
| | | QQKPGQPPKLLMYWASTRESGVPDRFSGSGSGTDFSLTISS |
| | | LQAEDVAVYYCQQYYNPPWTFGQGTKVEVK |
| 29. | Convergent CDRH3 | CAKGGATAAVFDSW |
| | from Ebola vaccine | |
| | cluster | |
| 30. | Convergent CDRH3 | CAKGGAAAAVFDSW |
| | from Ebola infection | |
| | cluster | |
| 31. | Convergent CDRH3 | CAKGAGAAAVFDHW |
| | from HIV infection | |
| | cluster | |
| 32. | ATL5170 HC | AAGCTTGCCGCCACCATGGGCTGGTCCTGCATCATCCTG |
| | Nucleotide sequence | TTTCTGGTGGCTACCGCTACCGGCGTGCACTCTCAGGTTC |
| | of full product gene | AGCTGGTTGAATCTGGCGGCGGAGTGGTGCAGCCTGGCA |
| | from 5′ cloning | GATCTCTGAGACTGTCTTGTGCCGCCTCCGGCTTCACCTT |
| | site to | CTCCAACTACGGAATGCACTGGGTCCGACAGGCCCCTGG |
| | 3′ cloning | CAAAGGATTGGAATGGGTCGCCGTGACCTCCTTCGACGG |
| | | CAAGACCAAGTACTACGCCGACTCCGTGAAGGGCAGATT |
| | | CACCATCTCTCGGGACAACTCCAAGAACACCCTGTACCTG |
| | | CAGATGAACTCCCTGAGAGCCGAGGACACCGCCGTGTAC |
| | | TACTGTGCTAAAGGCGGCGCTACAGCCGCCGTGTTGGAT |
| | | TCTTGGGGACAGGGCACACTGGTCACCGTGTCCTCTGCT |
| | | TCTACCAAGGGACCCAGCGTGTTCCCTCTGGCTCCTTCCA |
| | | GCAAGTCTACCTCTGGCGGAACAGCTGCTCTGGGCTGCC |
| | | TGGTCAAGGACTACTTTCCTGAGCCTGTGACCGTGTCTTG |
| | | GAACTCTGGCGCTCTGACATCTGGCGTGCACACCTTTCCA |
| | | GCTGTGCTGCAGTCCTCCGGCCTGTACTCTCTGTCCTCTG |
| | | TCGTGACCGTGCCTTCCAGCTCTCTGGGAACCCAGACCT |
| | | ACATCTGCAATGTGAACCACAAGCCTTCCAACACCAAGGT |
| | | GGACAAGAAGGTGGAACCCAAGTCCTGCGACAAGACCCA |
| | | CACCTGTCCTCCATGTCCTGCTCCAGAACTGCTCGGCGG |
| | | ACCTTCCGTGTTCCTGTTTCCTCCAAAGCCTAAGGACACC |
| | | CTGATGATCTCTCGGACCCCTGAAGTGACCTGCGTGGTG |
| | | GTGGATGTGTCTCACGAGGACCCAGAAGTGAAGTTCAATT |
| | | GGTACGTGGACGGCGTGGAAGTGCACAACGCTAAGACCA |
| | | AGCCTAGAGAGGAACAGTACAACTCCACCTACAGAGTGG |
| | | TGTCCGTGCTGACCGTGCTGCACCAGGATTGGCTGAACG |
| | | GCAAAGAGTACAAGTGCAAGGTGTCCAACAAGGCCCTGC |
| | | CTGCTCCTATCGAAAAGACCATCTCCAAGGCCAAGGGCC |
| | | AGCCTAGGGAACCCCAGGTTTACACCTTGCCTCCATCTCG |
| | | GGACGAGCTGACCAAGAACCAGGTGTCCCTGACCTGCCT |
| | | CGTGAAGGGATTCTACCCCTCCGATATCGCCGTGGAATG |
| | | GGAGTCTAATGGCCAGCCTGAGAACAACTACAAGACAAC |
| | | CCCTCCTGTGCTGGACTCCGACGGCTCATTCTTCCTGTAC |
| | | TCCAAGCTGACAGTGGACAAGTCCAGATGGCAGCAGGGC |
| | | AACGTGTTCTCCTGCTCCGTGATGCACGAGGCCCTGCAC |
| | | AATCACTACACACAGAAGTCCCTGTCTCTGTCCCCTGGCT |
| | | GATGAATTC |
| 33. | ATL_0005170_LC | AAGCTTGCCGCCACCATGGGCTGGTCCTGCATCATCCTG |
| | Nucleotide sequence | TTTCTGGTGGCTACCGCTACCGGCGTGCACTCCGATATC |
| | of full product gene | GTGATGACCCAGTCTCCTGACAGCCTGGCTGTGTCTCTG |
| | from 5′ cloning site to | GGCGAGAGAGCCACCATCAACTGCAAGTCCTCTCAGTCC |
| | 3′ cloning site | CTGCTGTACCGGCGGAACAACAAGAACTACCTGGCCTGG |
| | | TATCAGCAGAAGCCCGGCCAGCCTCCTAAGCTGCTGATC |
| | | TACTGGGCCTCCACCAGAGAATCTGGCGTGCCCGATAGA |
| | | TTCTCCGGCTCTGGCTCTGGCACCGACTTTACCCTGACAA |
| | | TCAGCTCCCTGCAGGCCGAGGATGTGGCCGTGTACTACT |
| | | GCCAGCAGTACTACAACCCTCCTTGGACCTTTGGCCAGG |
| | | GCACCAAGGTGGAAATCAAGCGGACAGTGGCCGCTCCTT |
| | | CCGTGTTCATCTTCCCACCTTCCGACGAGCAGCTGAAGTC |
| | | CGGCACAGCTTCTGTCGTGTGCCTGCTGAACAACTTCTAC |
| | | CCTCGGGAAGCCAAGGTGCAGTGGAAGGTGGACAATGCC |
| | | CTGCAGTCCGGCAACTCCCAAGAGTCTGTGACCGAGCAG |
| | | GACTCCAAGGACAGCACCTACAGCCTGTCCTCCACACTG |
| | | ACCCTGTCCAAGGCCGACTACGAGAAGCACAAGGTGTAC |
| | | GCCTGCGAAGTGACCCATCAGGGCCTGTCTAGCCCTGTG |
| | | ACCAAGTCTTTCAACCGGGGCGAGTGCTGATGAATTC |
| 34. | >ATL_0005155-003 | ATGGGCTGGTCCTGCATCATCCTGTTCCTGGTGGCTACC |
| | HC Nucleotide | GCCACCGGCGTGCACTCCCAAGTGCAACTAGTGGAAAGT |
| | sequence of full | GGTGGTGGTGTGGTGCAACCCGGACGCTCCCTCCGGCT |
| | product gene from 5′ | GTCTTGTGCTGCTTCTGGCTTCACCTTCTCCAACTACGGC |
| | cloning site to 3′ | ATGCACTGGGTCAGACAGGCCCCTGGAAAAGGCCTGGAA |
| | cloning site: | TGGGTGGCCGTGACCTCCTTCGATGGCAAGACCAAGTAC |
| | | TATGCCGACTCCGTGAAGGGCAGATTCACCATCTCCCGG |
| | | GACAACTCCAAGAACACCCTGTACCTGCAGATGAACTCCC |
| | | TGAGAGCCGAGGACACCGCTGTGTACTACTGCGCCAAGG |
| | | GCGGCGCTACCGCCGCCGTGTTCGACTCTTGGGGCCAG |
| | | GGCACACTGGTGACCGTGTCCAGCGCAAGTACTAAGGGT |
| | | CCAAGTGTGTTTCCACTGGCTCCCTCTAGCAAGTCCACCT |
| | | CTGGCGGCACCGCCGCCCTGGGCTGCCTGGTCAAGGAC |
| | | TACTTCCCCGAGCCCGTCACCGTGTCCTGGAACTCCGGC |
| | | GCTCTGACCTCCGGCGTGCATACCTTCCCTGCTGTGCTG |
| | | CAGTCTTCTGGCCTGTACTCCCTGTCCTCCGTCGTGACAG |
| | | TGCCTTCTAGCTCTCTCGGCACACAGACATACATCTGCAA |
| | | TGTGAACCACAAGCCTTCCAACACCAAAGTGGATAAGAAG |
| | | GTGGAACCTAAGTCCTGCGACAAAACCCACACCTGTCCTC |
| | | CTTGCCCTGCTCCTGAAGCCGCTGGCGGACCTTCTGTGT |
| | | TTCTGTTTCCCCCAAAGCCCAAAGACACCCTGATGATCTC |
| | | TCGGACACCTGAGGTGACCTGCGTGGTGGTGGATGTGTC |
| | | TCACGAGGACCCCGAAGTGAAGTTCAACTGGTACGTGGA |
| | | CGGCGTGGAAGTGCACAACGCCAAGACCAAGCCACGCGA |
| | | AGAGCAGTACAACTCTACCTACAGAGTGGTGTCCGTGCTC |
| | | ACAGTGCTGCACCAGGACTGGCTGAACGGAAAAGAGTAC |
| | | AAGTGCAAGGTCTCTAACAAGGCTCTGCCAGCCCCTATCG |
| | | AGAAGACCATCTCCAAGGCCAAGGGCCAACCTAGAGAGC |
| | | CTCAAGTGTACACCCTGCCTCCTAGCCGGGACGAGCTGA |
| | | CCAAGAACCAGGTTTCCCTGACCTGTCTGGTGAAAGGCTT |
| | | CTACCCCTCCGATATCGCCGTGGAATGGGAGTCCAACGG |
| | | CCAGCCTGAGAACAACTACAAGACCACCCCTCCTGTGCT |
| | | GGACTCCGATGGATCCTTCTTCCTGTATAGCAAGCTGACC |
| | | GTGGACAAGTCCAGATGGCAGCAGGGCAACGTGTTCAGC |
| | | TGTTCCGTGATGCACGAGGCCCTGCATAATCACTACACCC |
| | | AGAAGTCTCTGAGCCTGTCTCCTGGCAAGTGA |
| 35. | >ATL_0005155-003 | ATGGGCTGGTCCTGCATCATCCTGTTCCTGGTGGCTACC |
| | LC Nucleotide | GCCACCGGCGTGCACTCCGATATCGTGATGACTCAAAGT |
| | sequence of full | CCAGATAGTCTCGCTGTGTCCCTGGGCGAGAGAGCCACA |
| | product gene from 5′ | ATCAACTGCAAGTCCAGCCAAAGCCTGCTGTACCGGCGG |
| | cloning site to 3′ | AACAACAAGAACTACCTGGCCTGGTACCAGCAGAAACCTG |
| | cloning site: | GCCAGCCTCCTAAGCTGCTGATCTACTGGGCTTCTACCAG |
| | | AGAGTCTGGCGTCCCAGATAGATTCTCCGGCTCTGGCTC |
| | | CGGAACCGACTTCACCCTGACCATCTCCTCTCTGCAGGC |
| | | CGAGGACGTGGCCGTGTACTACTGTCAGCAGTACTATAAT |
| | | CCCCCTTGGACCTTTGGCCAGGGCACCAAGGTGGAAATC |
| | | AAGAGAACCGTGGCCGCTCCTTCTGTGTTCATCTTCCCTC |
| | | CTTCCGACGAGCAGCTGAAGTCCGGCACCGCTTCCGTGG |
| | | TGTGCCTGCTGAACAACTTCTACCCTAGAGAGGCCAAGGT |
| | | GCAGTGGAAGGTGGACAACGCCCTGCAGTCTGGCAACTC |
| | | CCAAGAGTCCGTCACCGAGCAGGACTCCAAGGACTCTAC |
| | | CTACTCCCTGTCCTCCACCCTGACACTGAGCAAGGCCGA |
| | | CTACGAGAAGCACAAGGTGTACGCCTGCGAGGTGACCCA |
| | | CCAGGGCCTGTCTTCTCCTGTGACCAAGTCCTTCAACAGA |
| | | GGCGAGTGCTGA |
| 36. | ATL4717 Hc full | QVQLVESGGGMVQPGRSLRLSCAASGFTFSIYGMHWVRQAPG |
| | | RGLEWVAVMSFDGKTKYYADSVKGRFTISRDNSKNTLYLQMNS |
| | | LRRVDTAVYYCAKGGATAAVFDSWGQGTLVTVSS |
| | | ASTKGPSVFPLAPSSKSTSGGTAALGCLVKDYFPEPVTVSW |
| | | NSGALTSGVHTFPAVLQSSGLYSLSSVVTVPSSSLGTQTYIC |
| | | NVNHKPSNTKVDKKVEPKSCDKTHTCPPCPAPELLGGPSVF |
| | | LFPPKPKDTLMISRTPEVTCVVVDVSHEDPEVKFNWYVDGV |
| | | EVHNAKTKPREEQYNSTYRVVSVLTVLHQDWLNGKEYKCKV |
| | | SNKALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| 37. | ATL4717 Lc full | DIVMTQSPDSLAVSLGERAAINCKSSQSLLYRSNSKNYLAWYQQ |
| | | QPGQPPKLLVYWASTRESGVPDRFSGSGSGTDFTLTISSLQAE |
| | | DVAVYYCQQYYNPPWTFGRGTKVEIKRTVAAPSVFIFPPSDEQ |
| | | LKSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| 38. | ATL4894 Hc full | QVQLVESGGGVVQPGRSLRLSCAASGFTFSIYGMHWVRQAPG |
| | | KGLEWVAVMSFDGKTKYYADSVKGRFTISRDNSKNTLYLQMNS |
| | | LRAEDTAVYYCAKGGATAAVFDSWGQGTLVTVSSASTKGPSV |
| | | FPLAPSSKSTSGGTAALGCLVKDYFPEPVTVSWNSGALTSG |
| | | VHTFPAVLQSSGLYSLSSVVTVPSSSLGTQTYICNVNHKPSN |
| | | TKVDKKVEPKSCDKTHTCPPCPAPELLGGPSVFLFPPKPKDT |
| | | LMISRTPEVTCVVVDVSHEDPEVKFNWYVDGVEVHNAKTKP |
| | | REEQYNSTYRVVSVLTVLHQDWLNGKEYKCKVSNKALPAPI |
| | | EKTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGK |
| 39. | ATL4894 Lc full | DIVMTQSPDSLAVSLGERATINCKSSQSLLYRSNSKNYLAWYQQ |
| | | KPGQPPKLLIYWASTRESGVPDRFSGSGSGTDFTLTISSLQAED |
| | | VAVYYCQQYYNPPWTFGQGTKVEIKRTVAAPSVFIFPPSDEQL |
| | | KSGTASVVCLLNNFYPREAKVQWKVDNALQSGNSQESVTE |
| | | QDSKDSTYSLSSTLTLSKADYEKHKVYACEVTHQGLSSPVTK |
| | | SFNRGEC |
| 40. | ATL4715 Hc full | EVQLVESGGGVVQPGRSLRLSCAASGFTFSRYGMHWVRQAPG |
| | | KGLEWVAVMSYDGSTKYYADSVKGRFAISRDNPKNTLFLQMNS |
| | | LRPDDTAVYYCAKGGAAAAVMDVWGKGTTVTVSSASTKGPSV |
| | | FPLAPSSKSTSGGTAALGCLVKDYFPEPVTVSWNSGALTSG |
| | | VHTFPAVLQSSGLYSLSSVVTVPSSSLGTQTYICNVNHKPSN |
| | | TKVDKKVEPKSCDKTHTCPPCPAPELLGGPSVFLFPPKPKDT |
| | | LMISRTPEVTCVVVDVSHEDPEVKFNWYVDGVEVHNAKTKP |
| | | REEQYNSTYRVVSVLTVLHQDWLNGKEYKCKVSNKALPAPI |
| | | EKTISKAKGQPREPQVYTLPPSRDELTKNQVSLTCLVKGFYP |
| | | SDIAVEWESNGQPENNYKTTPPVLDSDGSFFLYSKLTVDKS |
| | | RWQQGNVFSCSVMHEALHNHYTQKSLSLSPGK |
| 41. | ATL4715 Lc full | DIVMTQSPDSLTVSLGERATISCKSSQRLLYSSNNKNYLAWYQQ |
| | | KPGQPPKLLMYWASTRESGVPDRFSGSGSGTDFSLTISSLQAE |
| | | DVAVYYCQQYYNPPWTFGQGTKVEVKRTVAAPSVFIFPPSDE |
| | | QLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| 42. | ATL4894 scFV aa | QVQLVESGGGVVQPGRSLRLSCAASGFTFSIYGMHWVRQA |
| | | PGKGLEWVAVMSFDGKTKYYADSVKGRFTISRDNSKNTLYL |
| | | QMNSLRAEDTAVYYCAKGGATAAVFDSWGQGTLVTVSGGG |
| | | GSGGGGSGGGGSDIVMTQSPDSLAVSLGERATINCKSSQSL |
| | | LYRSNNKNYLAWYQQKPGQPPKLLIYWASTRESGVPDRFS |
| | | GSGSGTDFTLTISSLQAEDVAVYYCQQYYNPPWTFGQGTKV |
| | | EIK |
| 43. | ATL4894 scFV DNA | CAGGTTCAGCTGGTTGAAAGCGGTGGTGGTGTTGTTCAG |
| | | CCTGGTCGTAGCCTGCGTCTGAGCTGTGCAGCAAGCGGT |
| | | TTTACCTTTAGCATTTATGGTATGCATTGGGTTCGTCAGGC |
| | | ACCTGGTAAAGGTCTGGAATGGGTTGCAGTTATGAGCTTT |
| | | GATGGTAAAACCAAATATTACGCCGATAGCGTGAAAGGTC |
| | | GCTTTACCATTAGCCGTGATAATAGCAAAAATACCCTGTAC |
| | | CTGCAGATGAATAGTCTGCGTGCAGAGGATACCGCAGTG |
| | | TATTATTGTGCAAAAGGTGGTGCAACCGCAGCAGTTTTTG |
| | | ATAGCTGGGGTCAGGGCACCCTGGTTACCGTTAGCAGCG |
| | | GTGGCGGTGGTAGTGGTGGTGGCGGTAGCGGAGGTGGT |
| | | GGTTCAGATATTGTTATGACCCAGAGTCCGGATAGCCTGG |
| | | CAGTTAGCCTGGGTGAACGTGCAACCATTAATTGTAAAAG |
| | | CAGCCAGAGCCTGCTGTATCGTAGCAATAACAAAAACTAT |
| | | CTGGCATGGTATCAGCAGAAACCGGGTCAGCCTCCGAAA |
| | | CTGCTGATTTATTGGGCAAGCACCCGTGAAAGTGGTGTTC |
| | | CGGATCGTTTTAGCGGTAGTGGTAGCGGCACCGATTTTAC |
| | | CCTGACCATTAGCAGTCTGCAGGCAGAGGACGTTGCAGT |
| | | CTATTACTGTCAGCAGTATTACAATCCGCCTTGGACCTTTG |
| | | GCCAGGGCACCAAAGTTGAAATCAAA |

TABLE 1
Guide to selected antibodies of the disclosure.
Sequence ID numbers refer to the sequences
in Table 0.

| | Heavy Chain SEQ ID NOs. | | | | | Light chain SEQ ID NOs. | | | | |
|---|---|---|---|---|---|---|---|---|---|---|
| | (amino acid) | | | | | (amino acid) | | | | |
| Antibody | CDRH1 | CDRH2 | CDRH3 | VH | Full | CDRL1 | CDRL2 | CDRL3 | VL | Full |
| ATL4717 | 1 | 2 | 3 | 7 | 36 | 4 | 5 | 6 | 8 | 37 |
| ATL4894 | 1 | 2 | 3 | 10 | 38 | 9 | 5 | 6 | 11 | 39 |
| ATL5155 | 12 | 13 | 3 | 15 | 20 | 14 | 5 | 6 | 16 | 21 |
| ATL5170 | 12 | 13 | 17 | 18 | 22 | 14 | 5 | 6 | 19 | 23 |

### EXAMPLES

### Example 1—Methods

Analysis of public antibody repertoire data to identify convergent cluster Antibody sequences from the Ebola vaccine, Ebola vaccination, and HIV infection cohorts were extracted from the publications, and compared by eye. One CDR3 was identified that had similar variants across all three studies (not more than 3AA mismatches).

Alchemab's PDAC, Prostate cancer, and Melanoma cohorts were subsequently searched for the presence of the ATL5170 CDR3 sequence. 11 additional sequence variants were discovered across five individuals.

### CFH ELISA

Nunc maxisorp plates were coated with 1 ug/ml of recombinant human CFH (rhCFH), recombinant murine CFH (rmCFH) or irrelevant protein (lysozyme). 12 point antibody titrations of human IgGs ATL04717, ATL04830 (isotype control) and OX24 mouse anti-human CFH (Invitrogen MA 1-70057) were used with max concentration 10 ug/ml. Secondary anti-Human HRP (Invitrogen #62-8420; 1:2000) and anti-mouse HRP (Abcam #ab67889; 1:10,000) were used.

### Immunofluorescent Staining for CD11b and C3d in B16F10 Melanoma Tumours

C57BL/6 mice were inoculated subcutaneously with 2×105 B16F10 melanoma tumour cells. When MTV reached approximately 70-150 m3, IP dosing at 10 mg/kg with ATL4717 or a non-reactive anti-human IgG1 control, was initiated. Each mouse received 3 doses in total, 48 hours apart. Tumour tissues were snap frozen, sectioned onto slides, and fixed using 4% PFA/10% Neutral buffered formalin (Sigma-Aldrich). Slides were blocked (3% CS, 1% BSA, 0.3M Glycine (Alfa Aesar) in PBS. C3d was detected using rabbit anti-human C3d (Bioss Ltd (BS-4877R) 1:50 Secondary staining was completed using Donkey Anti-rabbit AF647 (Biolegend, A-31573) 1:300. Slides were imaged using the ImageXpress PICO imaging system (molecular devices) at 10× magnification. Section images were then analysed using CellReporterXpress imaging software, 2-channel cell scoring option. A paired t-test was then applied with an n=7 (GraphPad). IP=intraperitoneally; kg=kilogram; mg=milligram; MTV mean tumour volume; Human Immunoglobulin G1.

### A549 Spheroid Culture

A549 cells were seeded at 0.25 10⁵ cells/ml in 100 microlitre seeding volume in ultra-low attachment plates in RPMI medium (Gibco)+10% FBS. Cells were centrifuged at 125 g for 10 mins, then grown for 4 days at 37 C° 5% CO2, imaging every 4 hours in Incucyte instrument (Sartorius). On Day 4, media was removed and replaced with appropriate treatments (Media+Normal human serum (NHS; Complement Tech) plus/minus antibody ATL4717 at 50 microg/ml) and incubated for a further 6 days, imaging every 4 hrs. Data was analysed using Incucyte software Spheroid module to derive spheroid size.

### Monocyte Viability, Differentiation, and Cytokine Production

PBMCs were isolated from a LRS (Leucocyte Reduction System; NHS Blood and Transport) using Lymphopure (BioLegend 426201) density gradient reagent using standard methods. Monocytes were then isolated using Pan Monocyte Isolation kit (Miltenyi Biotec). Collected monocytes were resuspended at 1 million cells per ml and seeded into 96 well plates in 100 microliters. Cells were treated with CFH, and CFH+antibody (ATL4717 or isotype control), antibody alone (ATL4717 or isotype control) with or without LPS treatment. Untreated cells acted as controls.

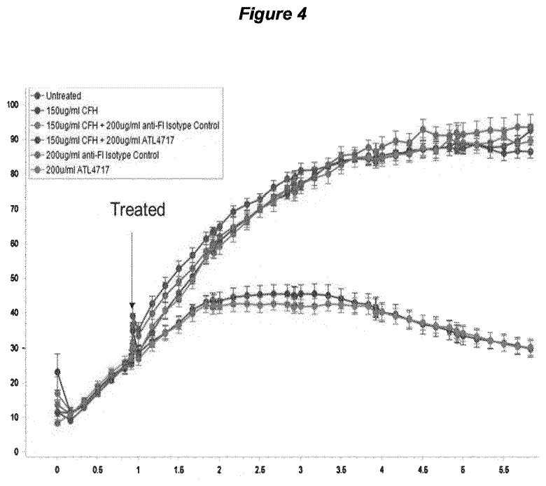

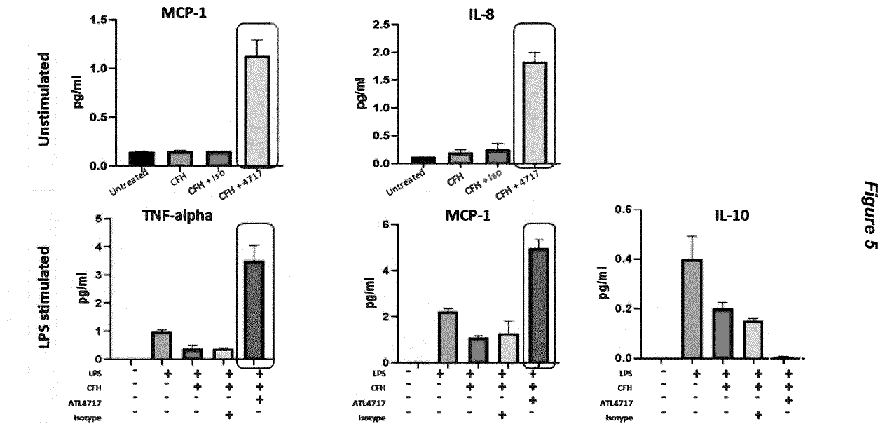

For cell viability (FIG. 4), cells were incubated (at 37° C., 5% CO2) with 1/500 cytotox green and imaged using Incucyte analysis (Sartorius) over a time course. Data shown is Cytotox green signal (higher signal indicates more cell death) For cytokine analysis (FIG. 5), cells were incubated for 48 hrs, with LPS added for the final 16 hrs. Supernatant was removed, centrifuged to remove residual cells (at 600 g 5 mins), before supernatant was analysed for cytokine concentrations using LegendPlex analysis (Biolegend).

For determining the effects of ATL5170 on monocyte differentiation and cytokine production, PBMCs were isolated from leucocyte cones by density gradient centrifugation (obtained from NHSBT). Monocytes were isolated from PBMC by negative selection (Human Pan Monocyte Isolation Kit; Miltenyi). 1×10⁵ monocytes were plated per well in a 96 well flat-bottom plate in RPMI 1640+10% heat inactivated foetal bovine serum (HI FBS)+50 ng/ml recombinant human GM-CSF (Biolegend). Monocytes were treated with or without 1, 10 or 100 μg/ml ATL5170 (Genscript) or ATL4892 (isotype control; Genscript) and incubated at 37° C., 5% CO2. Media was refreshed on day 3. Cell culture supernatants were harvested on day 6 for cytokine analysis. Cytokine production was assessed using the HU Essential Immune Response LEGENDplex Panel (Biolegend), as per the manufacturer's protocol.

### Monocyte/CD11b Phenotype Study

PBMCs were isolated from leucocyte cones using the ficoll standard method. Then, CD14 positive human primary monocyte cells were obtained by Magnetic-activated cell sorting (MACS, Miltenyi Biotec, Bergisch Gladbach, Germany) using the human Pan Monocyte Isolation kit (Miltenyi, Cat: 130-096-537). The isolated CD14⁺ monocyte cells were resuspended in Roswell Park Memorial Institute (RPMI) 1640 media+inactivated fetal bovine serum (FBS) at 3×10\hat{ }6 cells/ml and, 50 ul per well were distributed in a 96 well plate. The IgG1 isotype control and the anti-human CFH antibodies ATL_0004717, ATL_0005170 and ATL_0005170FcNull were resuspended at 500 nM concentration and added on top of the monocyte cells to a final concentration of 250 nM. Cells were incubated for 3 days at 5% CO2 and 37° C. After, cells were washed twice with PBS and stained with Live/Dead Fixable Violet (Thermo, Cat: 62248) and fluorophore conjugated antibodies anti human CD45, CD14 and CD11b (Biolegend, Cat: 304027, 367148, 301310). Flow cytometry was carried out using a FACS Symphony A1 flow cytometer (BD Biosciences, San Jose, Calif.). Percentage of CD45 positive cells was determined using Flow Jo software (Tree Star Inc., Ashland, Oreg.).

### THP-1 IL-8 Release Assay

THP-1 cells were seeded into flat bottom 96 well plates in RPMI+10% heat inactivated serum: 50 ul per well, 0.8×10\hat{ }6 cells/ml. 50 ul of antibody treatment was added at twice the desired final concentration. Cells were incubated for 48 hrs, 37 C 5% CO2, with LPS added in 10 ul for the final 8 hrs to give a final concentration of 110 ng/ml LPS

At the end of the incubation time, the media was removed and spun at 800 g for 5 mins and the supernatant taken for cytokine analysis using Biolegend LegendPlex Human Essential Immune Response Panel (13-plex) kit as per manufacturer's instructions.

### Monocyte Phagocytosis Study

PBMCs were isolated from leucocyte cones using the ficoll standard method. Then, CD14 positive human primary monocyte cells were obtained by Magnetic-activated cell sorting (MACS, Miltenyi Biotec, Bergisch Gladbach, Germany) using the human Pan Monocyte Isolation kit (Miltenyi, Cat: 130-096-537). The isolated CD14⁺ monocyte cells were resuspended in Roswell Park Memorial Institute (RPMI) 1640 media+inactivated fetal bovine serum (FBS) at 5×10\hat{ }5 cells/ml and, 50 ul per well were distributed in a 96 well plate. The IgG1 isotype control and the anti-human CFH antibodies ATL_0004717 and ATL_0005170 were prepared at 200 nM in RPMI+inactivated FBS containing pH sensitive pHrodo Red S. aureus Bioparticles Conjugate (Invitrogen, Cat: A10010) at 100 μg/mL. The solutions were added on top of the monocyte cells to a final concentration of 100 nM and incubated for 4 hours at 5% CO2 and 37° C. Phagocytosis was measured by light emission intensity (Ex/Em 560/585 nm) using the live cell imaging and software analysis system Incucyte S3 (Sartorius).

### Lymphocyte Shape Change

PBMC were isolated from leucocyte cones by density gradient centrifugation (obtained from NHSBT). CD4+ T cells were isolated from PBMC by negative selection (Human CD4+ T Cell Isolation Kit; Miltenyi). CD4+ T cells were frozen in Bambanker (Geneflow) at −80° C.

Frozen CD4+ T cells were rapidly thawed and rested overnight in RPMI 1640+10% HI FBS at 37° C., 5% CO2. After resting, 1.5×10⁵ CD4+ T cells were plated per well in a 96 well round-bottom plate in RPMI 1640+10% HI FBS, with or without 50 μg/ml ATL5170 (Genscript) or 50 μg/ml ATL4892 (Genscript; isotype control). Cells were cultured for 5 days at 37 C, 5% CO2. Cells were harvested, washed and resuspended in Dulbecco's PBS (DPBS) prior to performing flow cytometric analysis. Flow cytometry was carried out using a FACS A1 Symphony (BD Biosciences). The FSC-SSC profile was assessed as an indicator of T cell activation.

### CD4 Activation and IFNγ Release Essay

CD4+ T cell proliferation and IFNg production was assessed to monitor T cell activation following treatment with ATL5170.

PBMC were isolated from leucocyte cones by density gradient centrifugation (obtained from NHSBT). CD4+ T cells were isolated from PBMC by negative selection (Human CD4+ T Cell Isolation Kit; Miltenyi). CD4+ T cells were expanded for 6 days with anti-CD3/CD28-coated Dynabeads (Gibco) at a 1:1 ratio and 50 IU/ml recombinant human IL-2 (Peprotech). Dynabeads were removed and the cells rested overnight in RPMI 1640+10% HI FBS with 10 IU/ml IL-2 at 37° C., 5% CO2. Expanded CD4+ T cells were frozen in Bambanker (Geneflow) at −80° C.

Frozen expanded CD4+ T cells were rapidly thawed and rested overnight in RPMI 1640+10% HI FBS at 37° C., 5% CO2. After resting, CD4+ T cells were stained with CellTrace CFSE (Invitrogen), as per the manufacturer's guidelines, to monitor proliferation by dye dilution. 1×10⁵ CFSE-stained CD4+ T cells were plated per well in a 96 well round-bottom plate in RPMI 1640+10% HI FBS, with or without 1, 10 or 50 μg/ml ATL5170 (Genscript), ATL4892 (isotype control; Genscript) and/or Atezolizumab (anti-PDL1, Selleck Chemicals). Cells were cultured for 5 days at 37 C, 5% CO2. Cell culture supernatants were kept for cytokine analysis by ELISA (ELISA MAX Deluxe Set Human IFNg, Biolegend). Cells were harvested and washed in DPBS prior to staining for flow cytometry: cells were Fc-blocked (Human BD Fc Block, BD Biosciences) for 30 mins at room temperature and then stained with LIVE/DEAD Fixable Yellow Dead Cell Stain (Thermo) for 15 mins at 4° C. After washing in DPBS, cells were stained for surface markers (CD3-AF700, Biolegend; CD4-PE-Cy7, Biolegend) for 20 mins at 4° C. Cells were washed and resuspended in DPBS prior to running on a FACS A1 Symphony (BD Biosciences).

### Kinetic Studies

Biolayer interferometry (BLI, Octet 96Red) was used to measure binding kinetics of the interactions between anti-CFH antibodies and CFH. The CFH used for kinetics measurements was purified endogenous CFH was biotinylated and reduced with 20 mM dithiothreitol (DTT). An irrelevant protein was treated in the same way as a negative control reference. The CFH or irrelevant control ligands were each immobilised via the conjugated biotin onto a set of streptavidin (SA) sensor tips. Sensors were dipped into reference kinetics buffer (Phosphate buffered saline (PBS), pH 7.4, 0.05% Tween-20, 2% w/v not fat milk powder) to achieve a steady baseline. Association was measured over 300 s by dipping the sensors into an analyte concentration series of anti-CFH antibody (33.3-2.08 nM in kinetics buffer) as well as a no analyte control (kinetics buffer only). Dissociation was measured over 1500 s by dipping the sensors into kinetics buffer only. Baseline, association and dissociation steps were all measured with sensors shaking at 800 rpm and at a temperature of 23° C. Data was analysed with Data Analysis HT 11.1.0.25 software. Data from reference sensors and analyte control were subtracted. Data was corrected by aligning y-axis to average of baseline 5 s before association, inter step correction aligned to start of baseline and using Savitzky-Golay filtering. Data was fit to a 1:1 model using association and dissociation phases and kinetic rate constants are reported for the global fit where each curve R2 is >0.9.

### Example 2—Identification and Characterisation of Representative Antibody ATL4717

The present invention builds on the finding that convergent sequence clusters derived from the antibody repertoire of resilient groups of individuals may be used to identify disease-specific antibody sequences (Galson et al. 2020).

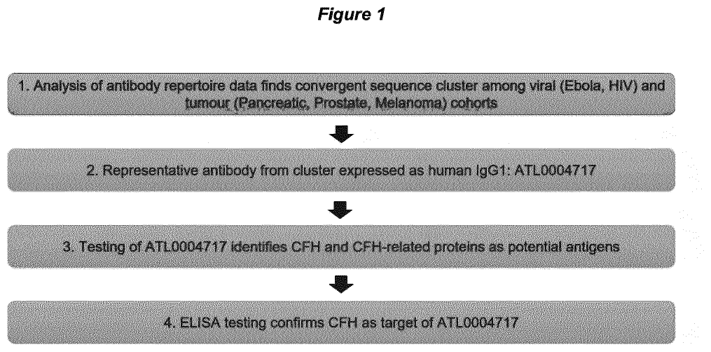

Publicly available datasets of antibody repertoires from Ebola vaccine, Ebola infection, HIV infection, and healthy control cohorts were deeply sequenced, and convergent CDRH3 were identified (FIG. 1) (see Table 2). From this convergent cluster, a representative antibody expressed as human IgG1: ATL0004717 (see Table 3) was identified.

TABLE 2
Convergent CDRH3 among Ebola vaccine,
Ebola infection, healthy control, and
HIV infection cohorts. Mismatched
amino acids are underlined.

| | | SEQ ID | |
|---|---|---|---|
| Study | CDRH3 | NO | Reference |
| Ebola | CAKGGAT | 29 | Ehrhardt et al 2019 |
| vaccine | AAVFDSW | | Nat Med 25: 1589 |
| Ebola | CAKGGAA | 30 | Davis et al 2019 |
| infection | AAVFDSW | | Cell 177: 1566 |
| HIV | CAKGAGA | 31 | Bowers et al 2014 |
| infection | AAVFDHW | | PLOS One 9: e81913 |

Representative antibody from cluster
expressed as human IgG1: ATL4717.
The convergent CDRH3 identified
in Table 3 is underlined.

| | | | | |
|---|---|---|---|---|
| ATL- | IgG1 | Antibody | Heavy | QVQLVESGGGMVQPGRS |
| 4717 | | from | chain | LRLSCAASGFTFSIYGM |
| | | Ebola | | HWVRQAPGRGLEWVAVM |
| | | vaccine | | SFDGKTKYYADSVKGRF |
| | | study | | TISRDNSKNTLYLQMNS |
| | | | | LRRVDTAVYYCAKGGAT |
| | | | | AAVFDSWGQGTLVTVSS |
| | | | | (SEQ ID NO: 7) |
| | | (Ehrhardt | Light | DIVMTQSPDSLAVSLGE |
| | | et al, | chain | RAAINCKSSQSLLYRSN |
| | | 2019) | | SKNYLAWYQQQPGQPPK |
| | | | | LLVYWASTRESGVPDRF |
| | | | | SGSGSGTDFTLTISSLQ |
| | | | | AEDVAVYYCQQYYNPPW |
| | | | | TFGRGTKVEIK |
| | | | | (SEQ ID NO: 8) |

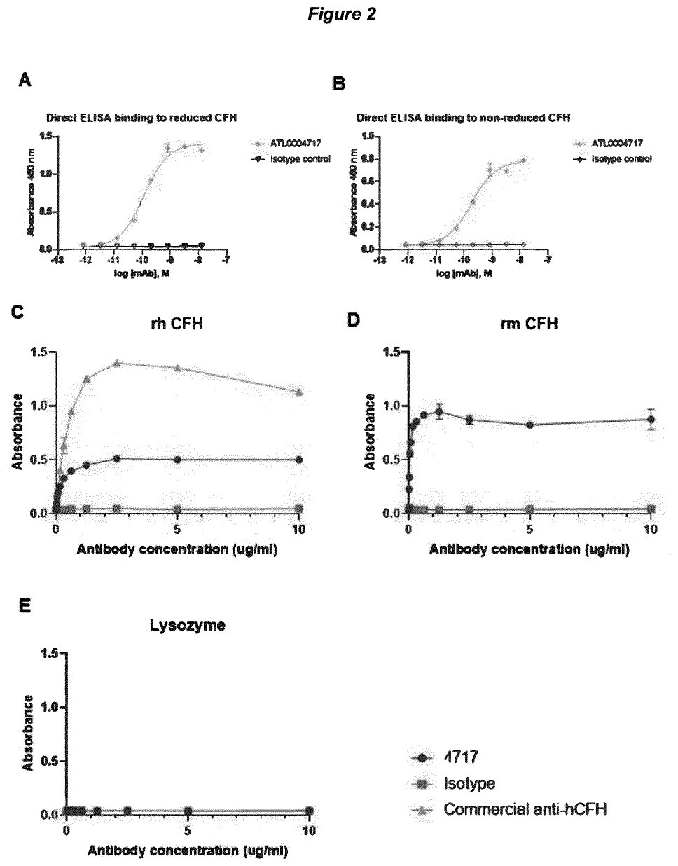

Target antigen screening was used to identify targets of ATL0004717, and ELISA confirmed CFH as a target of ATL4717 (FIG. 2). ATL4717 was found to bind both human and murine CFH (FIGS. 2 C and D).

CFH regulates the alternative pathway of the complement system (Whaley and Ruddy, 1976). It binds to C3b and blocks the alternative pathway of complement (Pangburn et al., 2000). By inhibiting alternative complement activation, CFH protects tumour cells against immune attack and has direct immunosuppressive activity (Ajona et al., 2004; Smolag et al., 2020; Nissila et al., 2018). CFH is upregulated in non-small lung cancer cell (NSLC) cell lines and tumour tissues (Ajona et al., 2004), and it is prognostic of a poor outcome in adenosarcoma (Versano et al., Clin Exp Immunol 1998; Cui et al., Int J Oncology 2011). It was therefore of interest to investigate whether ATL4717 is capable of inhibiting CFH function on tumour cells and promoting anti-tumour immunity.

To validate the importance of anti-CFH antibodies from a tumour perspective, internal databases as well as public datasets were searched for anti-CFH antibodies (data not shown). These searches led to the identification of these anti-CFH antibodies in tumour cohorts and in viral cohorts.

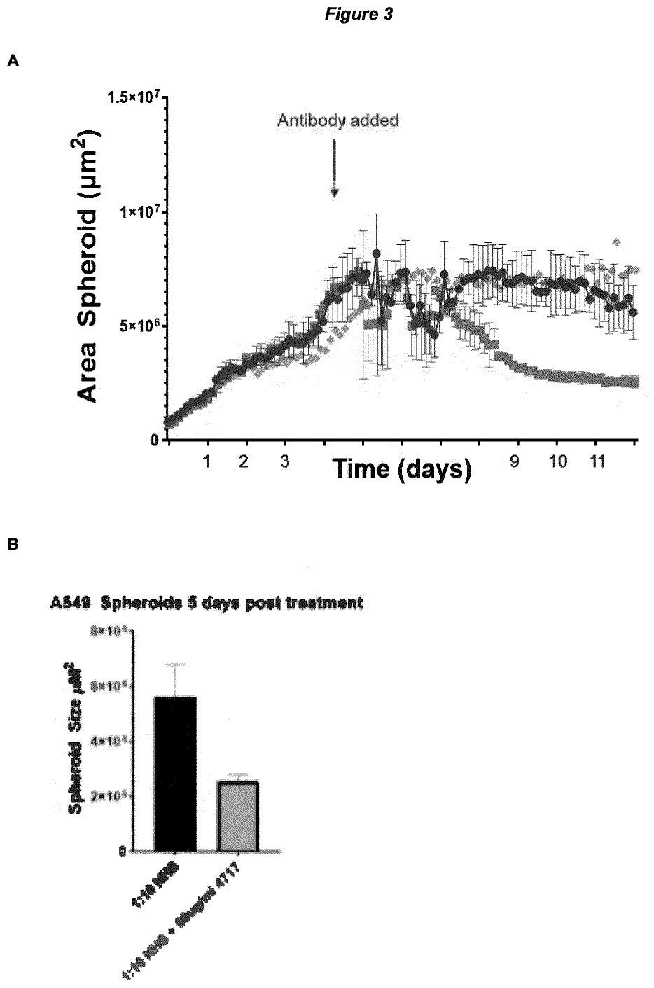

A 3D spheroid culture of A549 cells, which have been shown to express CFH (Yoon et al., 2019), an in vitro solid tumour model, was used to investigate whether CFH binding by ATL4717 is capable of killing tumour cells. After establishment of the cells as growing tumour spheroids, ATL4717 plus normal serum (red line, FIG. 3) or a serum control sample (orange line, FIG. 3) were added to the cells. The normal serum provides a source of complement proteins. FIG. 3 shows that addition of ATL4717 to the culture results in increased tumour cell killing as demonstrated by a decrease in spheroid area.

Next, it was investigated which types of immune cells could potentially be mobilised by inhibition of CFH through ATL4717. ATL4717 was found to increase monocyte survival (FIG. 4) and alter monocyte cytokine production (FIG. 5). In particular, monocytes cultured in the presence of ATL4717 showed increased expression of the pro-inflammatory cytokines MCP-1 and IL-8. LPS stimulation resulted in an even more pronounced increase in MCP-1 and TNF-α concentration, as well as a decrease in IL-10 concentration in monocytes cultured with ATL4717. Notably, addition of polymyxin B, which inhibits LPS, did not change these results (FIG. 5). These data indicate that the antibody-mediated alteration in cytokine production was not driven by LPS stimulation but rather by ATL4717.

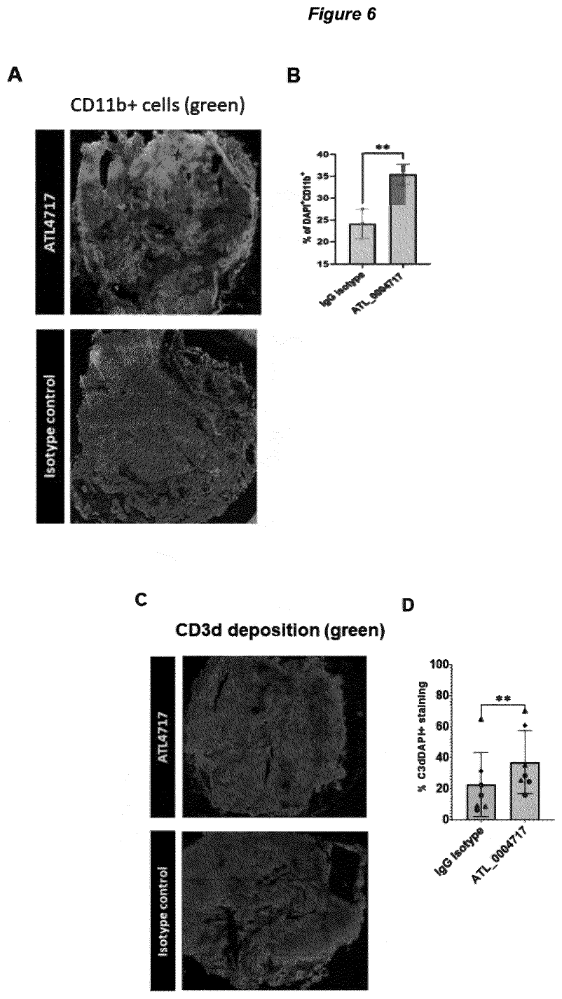

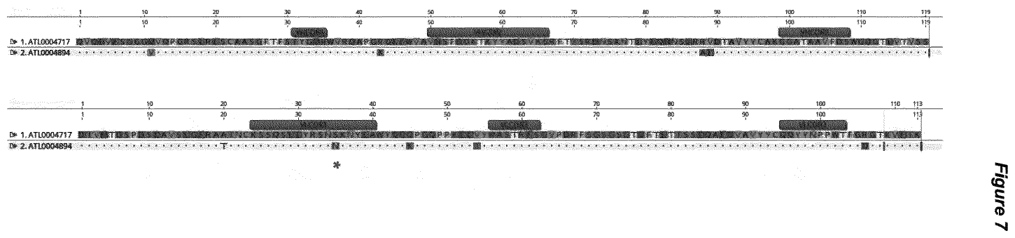

The in vivo effects of ATL4717 were investigated in B16/F10 syngeneic mice, a murine melanoma model. B16/F10 syngeneic mice treated with ATL4717 showed increased immune cell infiltration into tumour tissues as indicated by the significant increase in Cd11b+ cells in tumours compared with isotype controls (FIGS. 6A and B). In addition, treatment with ATL4717 resulted in increased C3d deposition in tumours compared with mice receiving the isotype control antibody (FIG. 6C). This suggests that ATL4717 is capable of relieving CFH-mediated inhibition of the alternative complement pathway.

In summary, ATL4717 increases tumour cell killing, promotes the production of pro-inflammatory cytokines in monocytes, drives infiltration of CD11b+ cells into tumours, and increases C3d deposition.

### Example 3—Optimisation of ATL4717-Generation and Optimisation of ATL4894

Having established that ATL4717 has potent anti-tumour effects, the antibody was further optimised to enhance its pharmacokinetic properties.

To reduce immunogenicity of the antibody, the framework regions of ATL4717 were reversed to germline encoded V-gene and J-gene sequences (so-called germlining) (FIG. 6, n=8; 4 in VH, 4 in VL). In addition, because NS deamidation sites are known sequence liabilities this site was also removed (see asterisk in FIG. 7). This initial optimisation step resulted in an antibody with higher yield and higher stability as indicated by the retention of 81.29% monomer of ATL4894 at 40° C. after two weeks compared with only 39.51% for ATL4717 (Tables 4 and 5), ATL4984.

TABLE 4
Yield of indicated mAb in 80 ml HEK, in mg.

| mAb | Yield from 80 ml HEK expression, mg |
|---|---|
| ATL0004717 | 0.2 |
| ATL0004894 | 12 |

TABLE 5
Two-week stability study for indicated
mAbs at indicated temperatures.

| mAb | % Monomer |
|---|---|
| ATL0004717, 4° C. | 98.15 |
| ATL0004717, 20° C. | 96.78 |
| ATL0004717, 40° C. | 39.51 |
| ATL0004894, 4° C. | 96.19 |
| ATL0004894, 20° C. | 95.40 |
| ATL0004894, 40° C. | 81.29 |

Given its excellent yield and stability, ATL4894 was taken forward as a template for further optimisation.

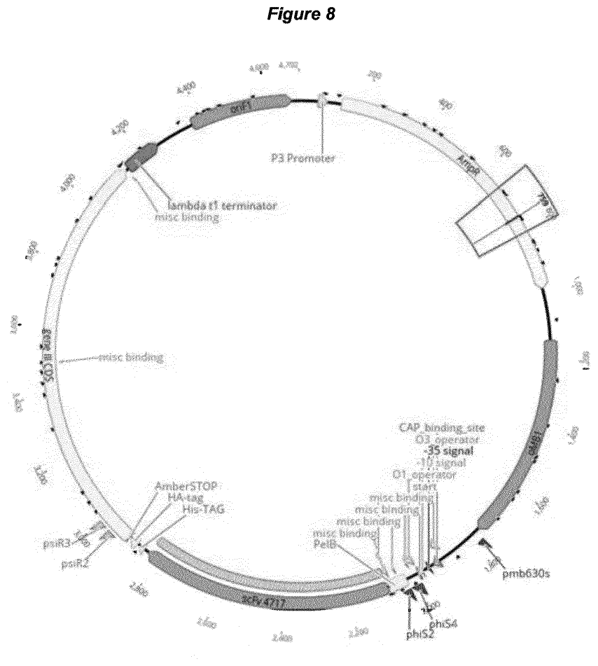

The scFv sequence ATL4984 was amplified by error prone PCR using an error prone polymerase (e.g. Taq). This introduced random mutations along the scFv sequence, producing a library of variants that can be used to identify variants with desired binding properties. The variants were subsequently cloned into a phage display vector (FIG. 8).

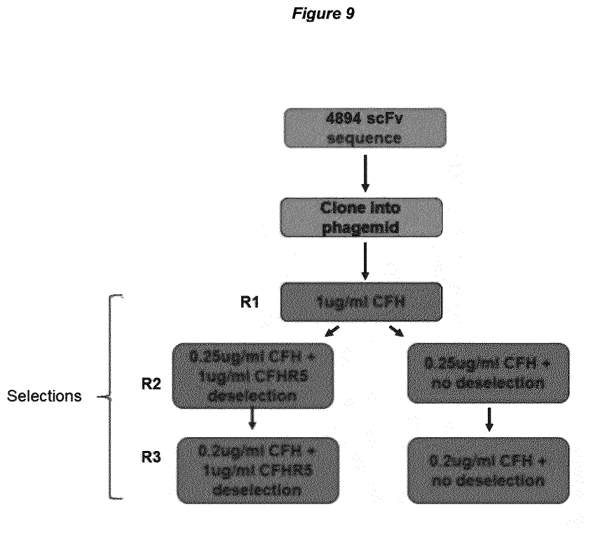

Two methods were used for the cloning step; (i) homology cloning resulting in a library size of approximately 1×10⁸ (using HiFi assembly, Gibson cloning), and (ii) restriction digest cloning resulting in a library size of approximately 1×10⁶. Phages were prepared from each library, and phage display allowed for antibodies that bind to CFH to be isolated from a larger library by performing selection based on antigen binding. Three rounds of selections were performed on the phage preparations. The first selection involved addition of CFH, followed by adding selection pressure by decreasing the CFH concentration, and reducing CFH and CFHR5 (FIG. 9).

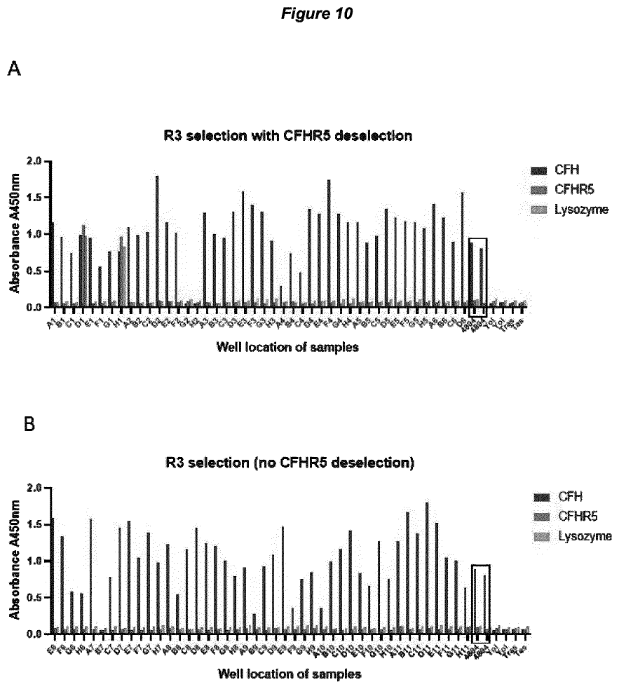

To test whether the selections were successful, colonies were picked from the output of the third selection round and ELISA was performed on the phages to look for binding to reduced CFH, reduced CFHR5, and lysozyme (control) (FIG. 10). Colonies that showed the desired binding profile, that is high binding signal to CFH and weak/no binding to CFHR5 were selected and sequenced and their amino acid sequences were compared to ATL4894. Sequencing confirmed the presence of framework-only mutations and CDR mutations in these samples (data not shown). The sequences were used to generate mAbs with variant VH and VL sequences.

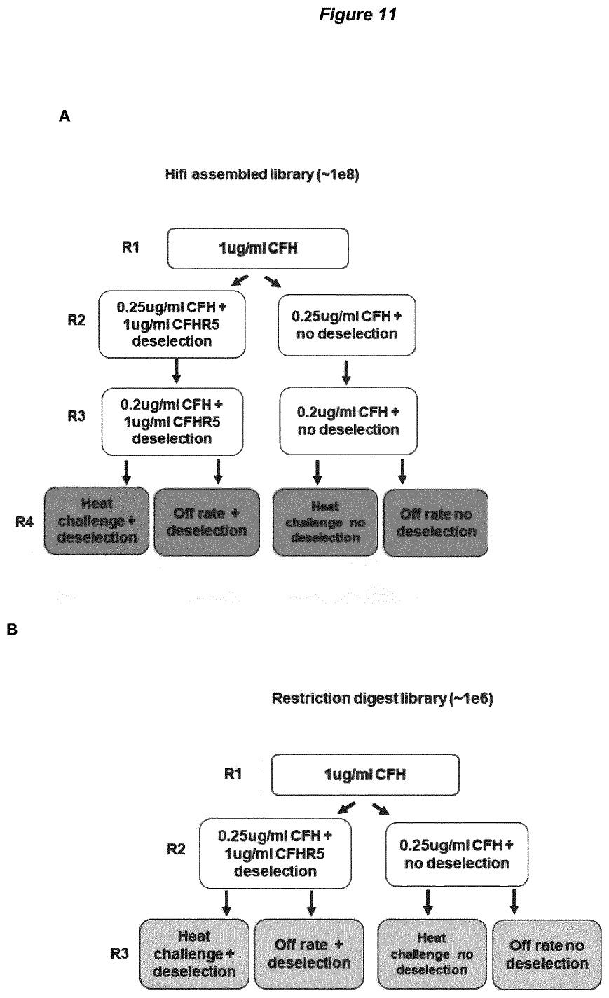

To obtain clones with both high affinity and high stability further selections were carried out (FIG. 11).

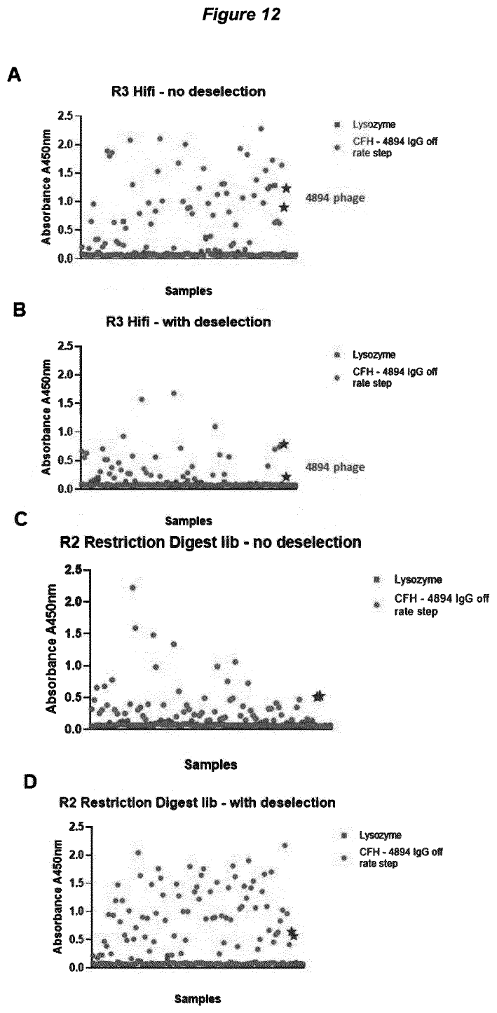

Off-rate ELISA experiments were carried out to identify high affinity clones, with slow off-rates. Off-rate ELISAs involved incubating the phages displaying the ATL4894 variants with immobilised reduced CFH or lysozyme as control. After a 1-hour incubation at room temperature to allow phage to bind to CFH, 3 washes were performed with PBS/0.1% Tween. To increase the off-rate stringency, a molar excess of ATL4894 lgG (at 20 μg/ml in 50 ul PBS/1% BSA was added to the ELISA plate for 30-40 minutes at room temperature. Any phage dissociating from CFH would be prevented from re-binding by the excess of 4894 IgG, allowing discrimination on the basis of off-rates. Following 3 washes in PBS (no Tween), phage binding was then determined using anti-M13-HRP secondary antibody (FIG. 12). The mAbs with the slowest off-rates bind strongly to the immobilised CFH and remain bound in the presence of the 4894 lgG added. Off-rate hits were identified and sequenced in order to identify mutations.

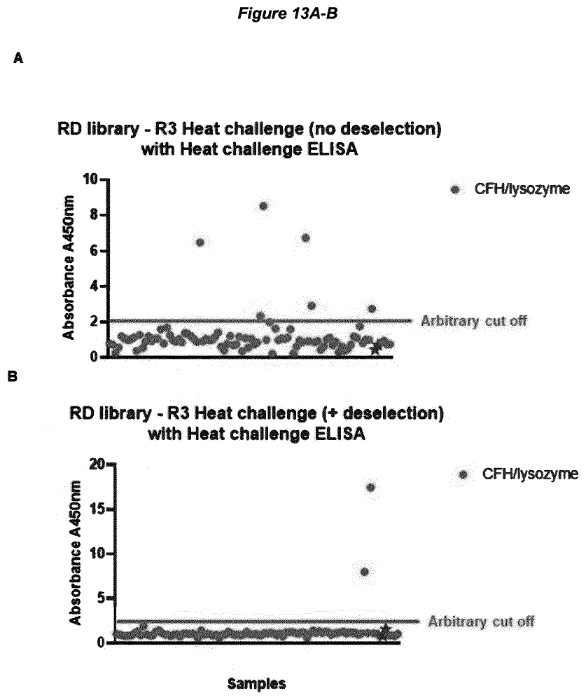

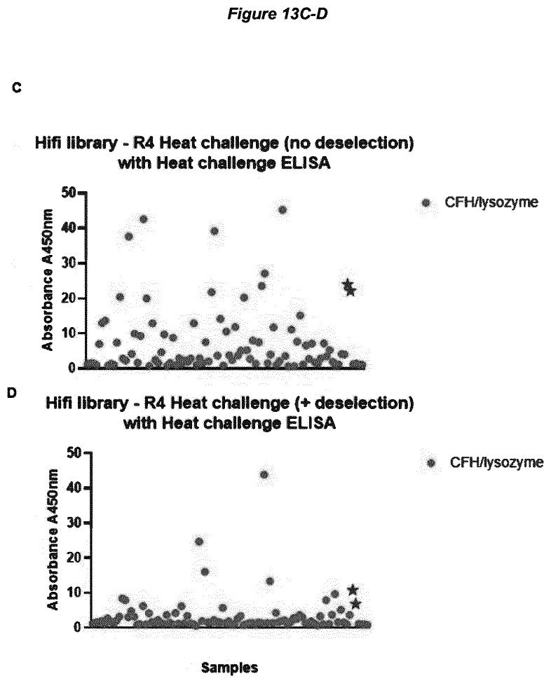

To identify mutants with higher stability than ATL4894, a heat challenge ELISA was used. Heat challenge ELISA was initially performed with a 70° C. pre-incubation pf phage but binding to CFH was quickly lost (data not shown). The heat challenge was then trialled at 50° and 60° C. for 0, 1, 2, 4, or 8 minutes prior to performing a phage ELISA to detect binding to reduced CFH or lysozyme (control) (data not shown). A heat challenge at 60° C. for 8 minutes resulted in approximately 50% reduction in binding signal for ATL4894 and these conditions were used to test variants (FIG. 13). Samples that showed good heat stability (above threshold/CFH/lysozyme signal >6) were sequenced and analysed for further mutations.

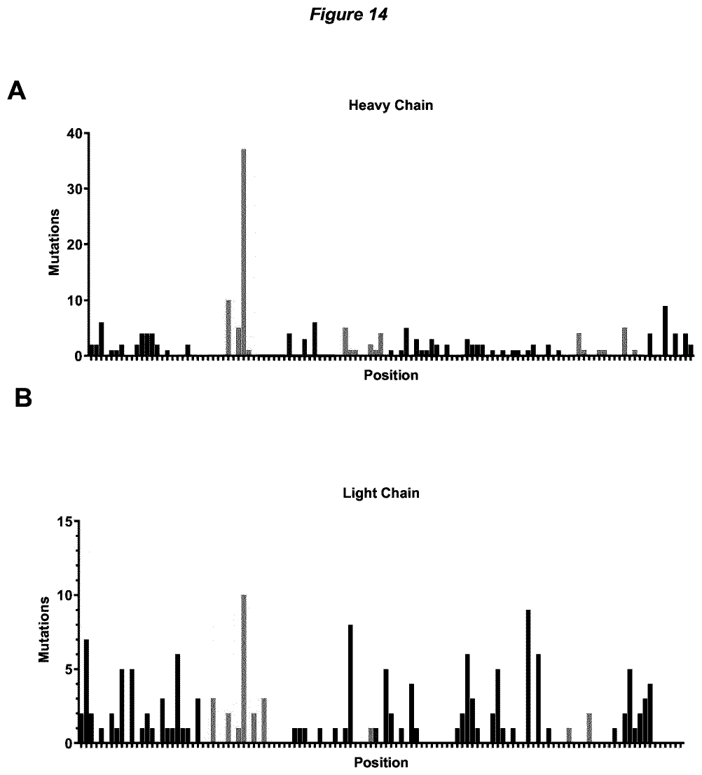

ELISA-positive clones from the various selection arms were sequenced, CDR sequences were identified according to IMGT definitions and each CDR sequence was clustered to identify variants and hotspots. A summary of CDR and framework mutations is shown in FIG. 14.

Clones with high frequency CDR changes across all CDRs that are different from 4894 were selected and three VH CDR sequences were identified (VH CDR1 (136N; frequency=13), VH CDR2 (M56T; frequency=5), VH CDR3 (F115L; frequency=5)); three VL CDR mutations were identified (VL CDR1 (S33G; frequency=4), VL CDR1 (S33R; frequency=3), VL CDR3 (P115T; frequency=2)). Five framework mutations were identified VH FW2 (L50P; frequency=6), VH FW3 (S70G; frequency=3), VH FW4 (L123Q; frequency=7), VL FW1 (L11Q; frequency=5), VL FW3 (E68V; frequency=3). Framework mutations were limited in lead antibodies in order to stay close to germline sequence and maintain biophysical properties of 4894 and multiplex optical imaging (IBEX) compatibility. These mutations were combined to eventually give rise to 10 lead mAbs.

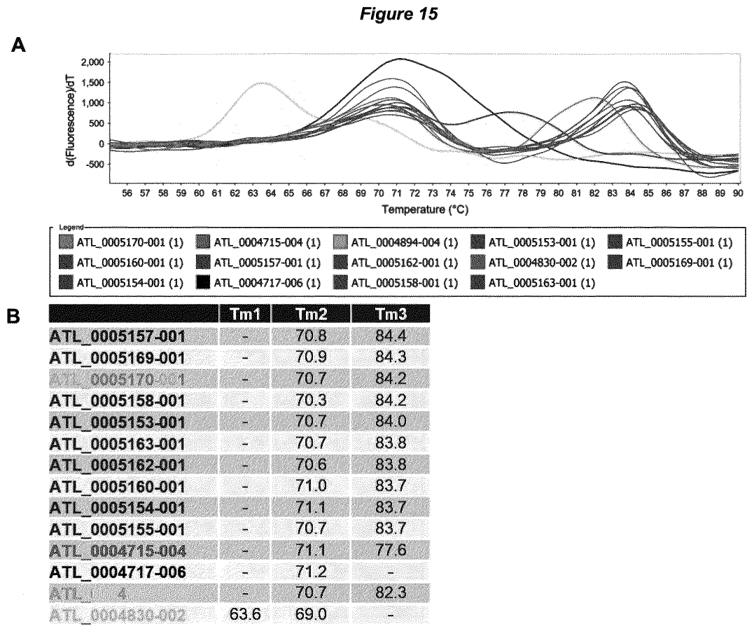

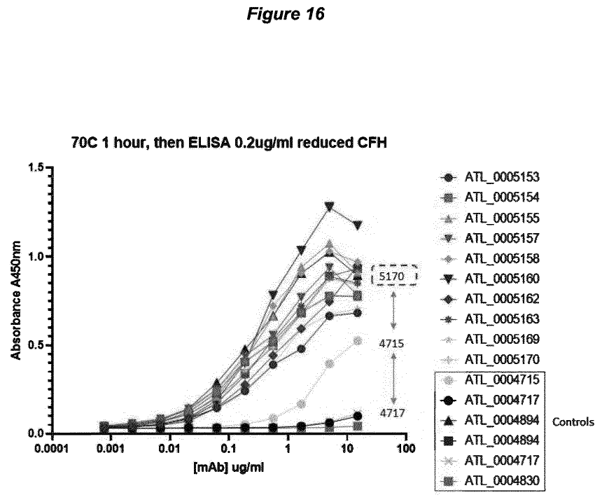

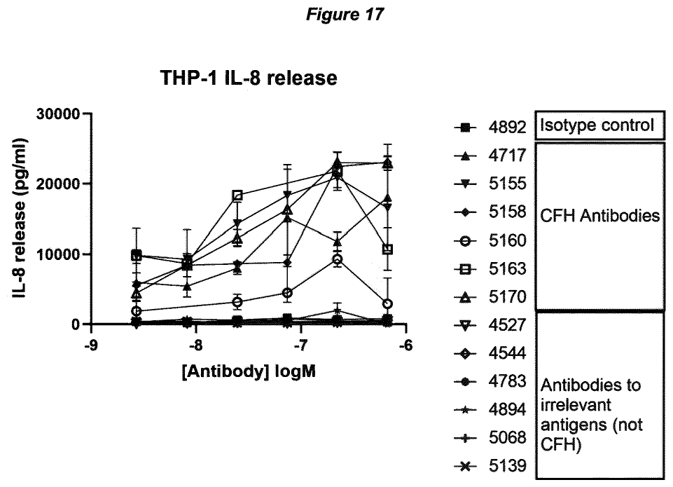

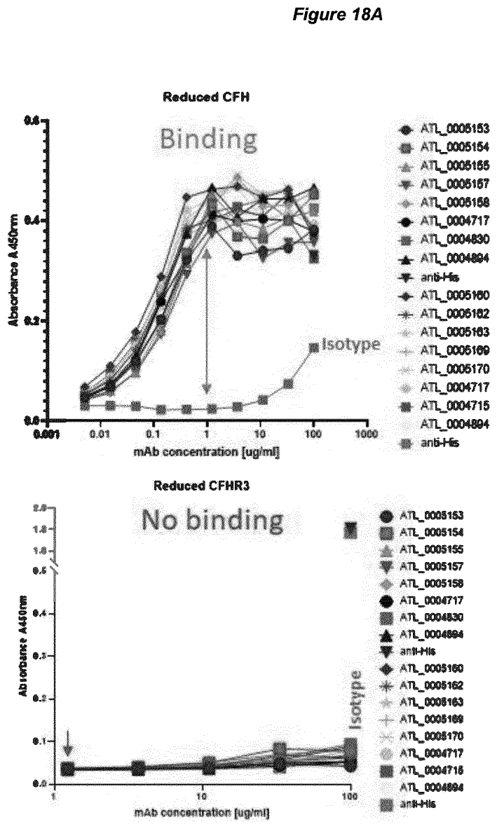

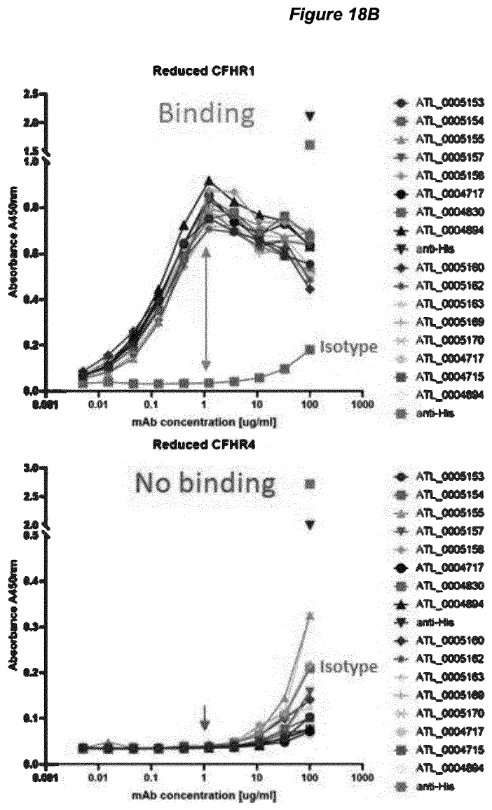

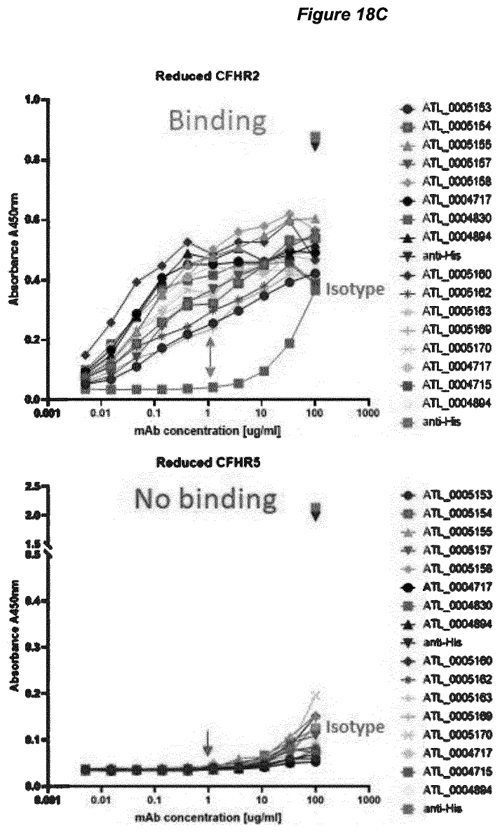

mAbs were further tested to identify the clone that (i) has optimal thermostability compared with the parent mAb ATL4717 and ATL4715 (FIG. 15); (ii) retains CFH binding after heating to 70° C. for one hour (FIG. 16); (iii) is active in a THP-1 monocyte IL-8 release assay (FIG. 17), and (iv) does not bind CFHR5 (FIG. 18).

FIG. 15 shows the melting curves observed from different domains of the IgGs. Tₘ1 was only present for the low stability isotype control mAb ATL4830. Tₘ2 values were comparable for most mAbs. Tₘ3 values had a broad range with clear stability benefits for some variants following protein engineering.

FIG. 16 shows the results of a harsh heat challenge ELISA, wherein the mAbs were incubated at 70° C. for one hour prior to testing their binding to reduced CFH. These results show ATL5170 as one of the most stable mAbs. The results also highlight significant improvements of the lead variants over the parent mAb (ATL4717) and ATL4715 mAb, in terms of stability.

FIG. 17 shows that ATL5170 consistently and strongly stimulates IL-8 in a THP-1 cytokine release assay. Notably, the germlined parent sequence (ATL 4894) does not result in IL-8 secretion.

FIG. 18 shows that the lead mAbs binds to reduced CFH, reduced CFHR1, and reduced CFHR2. The lead mAbs do not bind to CFHR3, CFHR4, and CFHR5.

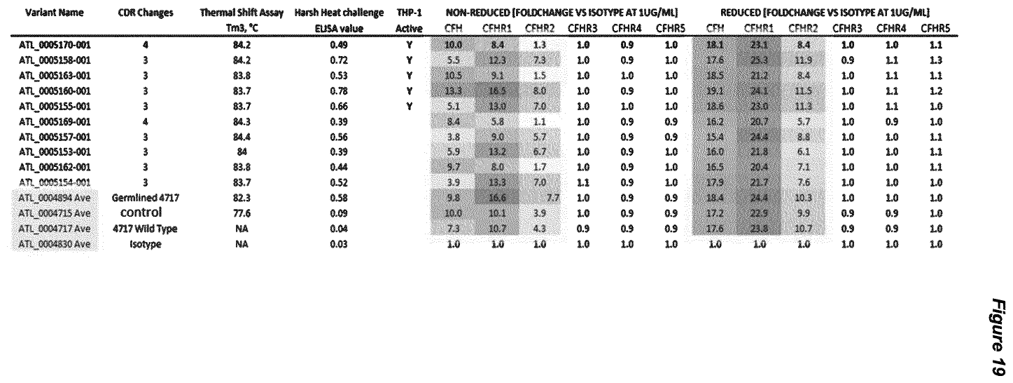

FIG. 19 shows a summary of these experiments and identifies ATL5170 as the lead candidate that meets the four goals described above.

### Example 4—ATL5170 Induces Strong Pro-Inflammatory Immune Responses

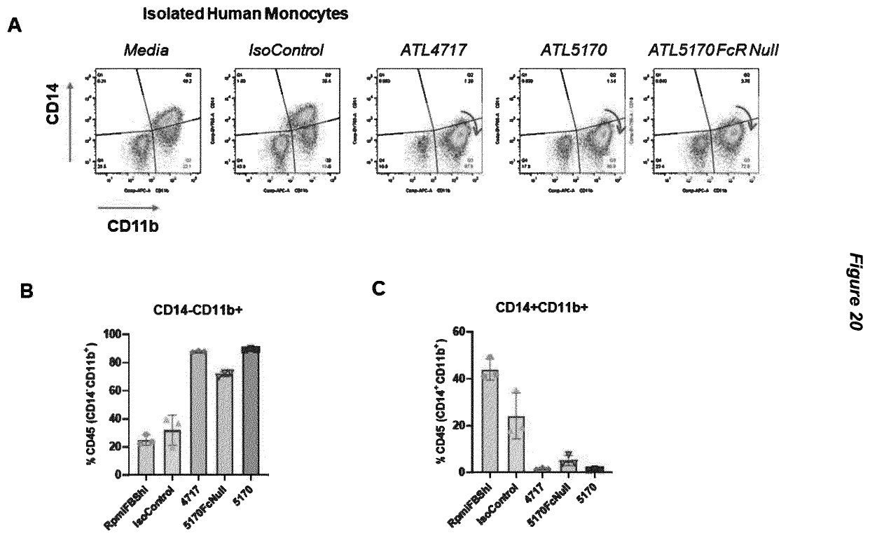

Given the ability of the ATL4717 antibody to increase monocyte survival and alter monocyte cytokine production (see FIGS. 4 and 5), the effects of ATL5170 and ATL4717 on monocyte differentiation into a phenotypic state resembling pro-inflammatory M1 macrophages were investigated (FIG. 20).

Isolated human monocytes were cultured in the presence of ATL4717 and ATL5170. After 3 days, cells were stained for CD14 (a marker associated with circulating monocytes) and CD11b (a marker upregulated in activated monocytes and macrophages) and analysed by flow cytometry (FIG. 20A). The frequency of CD11b+CD14-CD45+ cells was higher in cells treated with ATL4717 mAb or ATL5170 mAb, while the frequency of CD14+CD11b-CD45+ cells was lower (FIGS. 20A and B). Cells were also more adherent and activated in their morphology (data not shown). These data indicate that ATL5170 and ATL4717 mAb induce differentiation of monocytes into a cell phenotype more resembling an activated macrophage state. Notably, this differentiation into macrophages was independent of FcR engagement. An ‘FcRnull’ version of ATL5170 was made. This is an IgG1 format edited in the Fc domain to result in significantly reduced binding of the Fc portion of the antibody to Fc receptors, which are highly expressed on this cell type. Despite this antibody not engaging FcRs, a similar level of activation was observed, indicating this effect was not mediated via a Fc/FcR interaction. This could be via a direct effect of the antibody on CFH/C3R or other CFH binding partner on the surface of the cells, or via activation via increased C3d deposition.

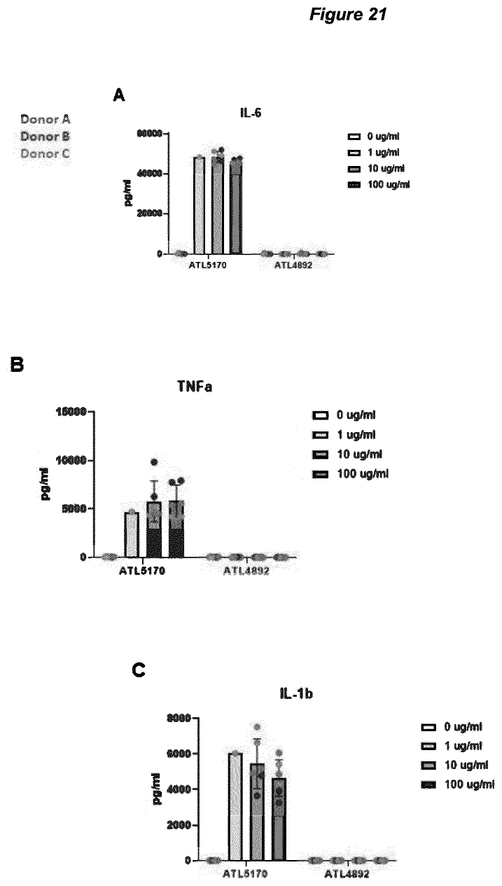

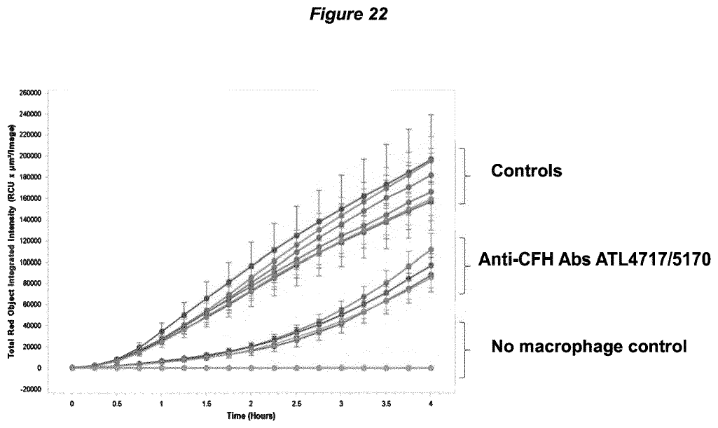

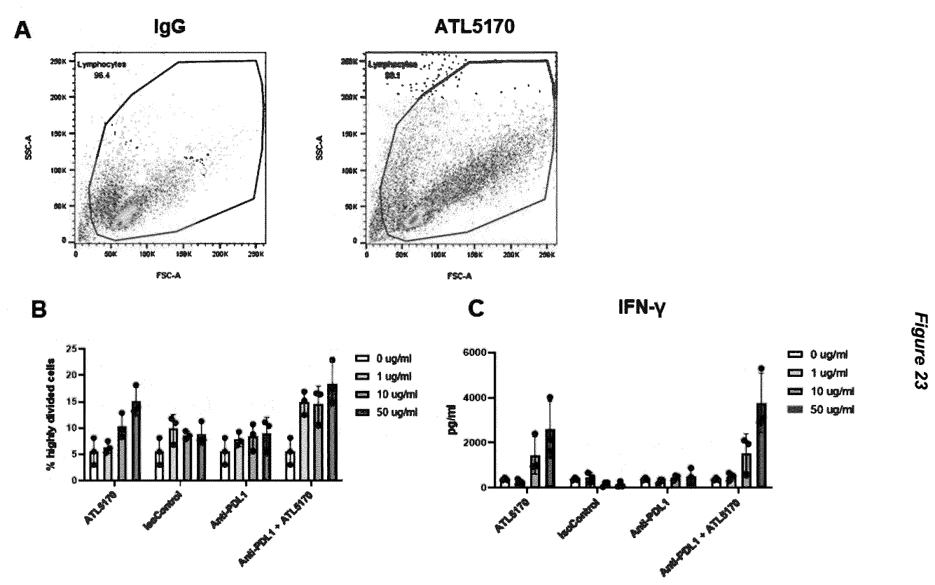

Next, the effect of ATL5170 on macrophage activation was tested by measuring the levels of the pro-inflammatory cytokines IL-6, TNF-α, and IL-1β. The lead mAb ATL5170 induced high levels of all three of these pro-inflammatory cytokines compared with the isotype control mAb (ATL4892) (FIG. 21). This pro-inflammatory cytokine signature is typical of activated, so-called “M1-like” macrophages, known to have potent anti-tumour activity (Duan et al., 2021). ATL5170 was also shown to reduce phagocytosis of pHRodo labelled bacteria by macrophages consistent with the pro-inflammatory M1-like cytokine signature observed (FIG. 22) (Duan et al., 2021).

Together, these data indicate that ATL5170 skews monocyte derived macrophages towards an activated, M1-like state.

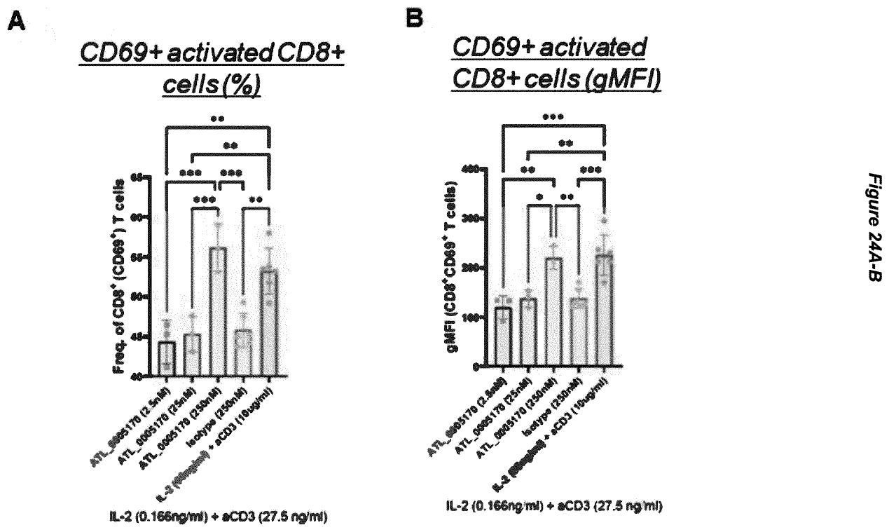

To investigate the effect of ATL5170 on CD4 T cells, ATL5170 or an isotype IgG control was added to isolated CD4 T cells (FIG. 23). CD4 T cells treated with ATL5170 showed a change in shape and granularity indicated by a shift in forward and side scatter by flow cytometry not observed in the control group (FIG. 23A), indicative of lymphocyte activation. This increased CD4+ activation was consistent with an increase in CD4 proliferation (FIG. 23B). CD4 cells also expressed significantly higher levels of the pro-inflammatory cytokine IFN-γ when treated with ATL5170 mAb without any co-stimulation (FIG. 23C). Notably, both CD4 cell proliferation (FIG. 23B) as well as IFN-γ secretion (FIG. 23C) were even more pronounced when cells were treated in the presence of ATL5170 and anti-PDL1, indicating that these two mAbs act synergistically.

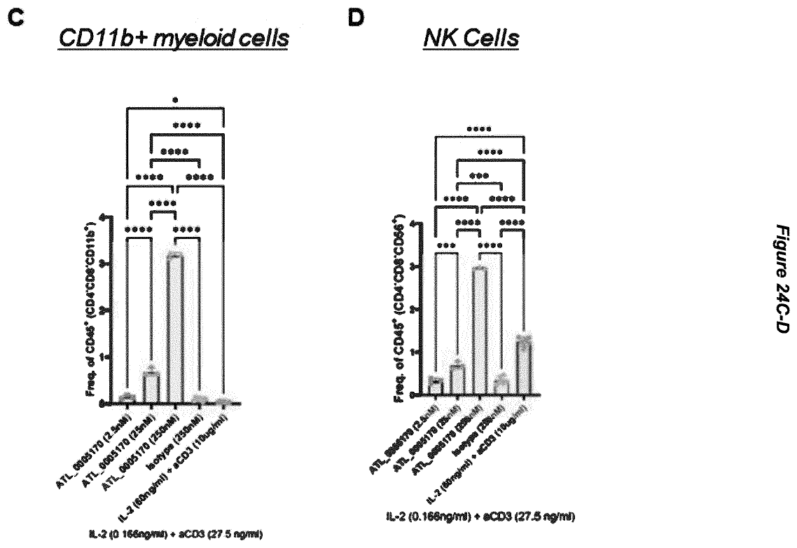

Finally, the effect of ATL5170 on CD8 T cells and NK cells, both known to play a prominent role in anti-tumour immunity, was tested by co-culturing PBMCs and PDAC10.02 tumour cells (FIG. 24). The expression of CD69 (a well-established activation marker for T lymphocytes) on CD8 T cells was significantly increased when cells were treated with ATL5170 (250 nM) when compared to isotype controls or cells cultured in IL-2 and CD3. This was to a similar level as the positive control where cells were cultured in high concentration of IL-2 and CD3 to maximally activate CD8 cells in the assay (FIG. 24A-B).

ATL5170 treatment also resulted in a significant increase in the percentage of NK cells and CD11b+myeloid cells even at the lowest concentration of 2.5 nM (FIGS. 24 C-D).

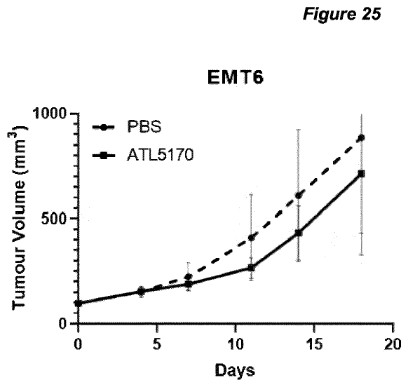

ATL5170 also inhibits tumour growth in vivo, as demonstrated by a dose dependent inhibition of tumour growth by ATL5170 in a EMT6-BALB/c syngeneic mouse tumour model (FIG. 25).

In summary, ATL5170 increased monocyte infiltration into the tumour and induces macrophage differentiation towards the pro-inflammatory M1 state. ATL5170 was further shown to activate CD4+ cells and synergise with an anti-PD1 antibody in this regard. ATL5170 activates CD8 T cells and increases the proportion of NK cells and myeloid cells in an in vitro tumour model. Finally, ATL5170 also inhibits tumour growth in vivo.

### Example 5—ATL5170 Demonstrates Classical Dose-Dependent PK

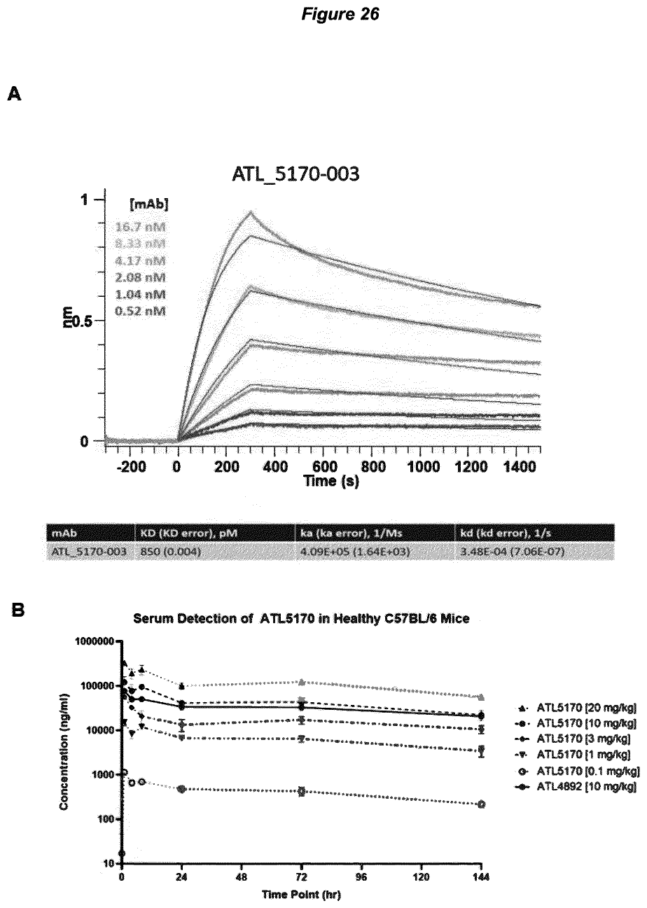

Kinetic analysis of ATL5170 shows association of the mAb with immobilised CFH at an affinity range of between KD 0.402-1.46 nM (FIG. 26A). The antibody kinetics could only be observed using surface immobilised CFH, and not in an alternative format using soluble phase CFH.

A dose range finding study indicates that i.v. administration of ATL5170 in mice results in a classical dose-dependent disposition equivalent to human IgG1 (ATL4892) (FIG. 26B). Advantageously, ATL5170 mAb does not bind to soluble CFH, as indicated by lack of accelerated antigen-mediated clearance. In addition, analysis of blood cell counts and clinical chemistry (including kidney markers) at 144 hours after a single administration did not show any toxicological results of concern in this preliminary analysis.

In summary, ATL5170 has an excellent PK profile and, while ATL5170 binds a conformational epitope of CFH when it is surface or cell-surface associated, it does not bind to CFH in serum. This cell-specific binding profile is advantageous as it avoids potential safety issues associated with circulating CFH depletion, such as lysis of blood cells, and reduces the anticipated antibody dose required.

### Example 6—In Vivo Efficacy Study of the ATL5170 in the Treatment of a Panel of 12 Syngeneic Models

The objective of this study was to evaluate the anti-tumour activity of the ATL5170 test compound in the treatment of a panel of 12 subcutaneous syngeneic models.

Experiments were performed in either BALB/c or C57BL/6 mice at 6-8 weeks at an estimated body weight of greater than 17 g. The 12 syngeneic cell lines were maintained in vitro with different medium (indicated in Table 6) at 37° C. in an atmosphere of 5% CO2 in air. The cells in an exponential growth phase were harvested and counted for tumour inoculation.

TABLE 6
Types of media used for different cell lines

| Cell Line | Medium |
|---|---|
| H22 | RPMI1640 + 10% FBS |
| EMT-6 | DMEM + 10% FBS |
| MC38 | DMEM + 10% FBS |
| B16F10 | DMEM + 10% FBS |
| Pan02 | RPMI1640 + 10% FBS |
| CT26 | RPMI1640 + 10% FBS |
| LL/2 | DMEM + 10% FBS |
| A20 | RPMI1640 + 10% FBS |
| Hepa1-6 | DMEM + 10% FBS |
| Renca | DMEM + 10% FBS |
| B16BL6 | RPMI1640 + 10% FBS |
| RM-1 | RPMI1640 + 10% FBS |

Each mouse was inoculated subcutaneously with the designated amount of tumour cells (as shown in Table 7) in 0.1 mL PBS for tumour development.

TABLE 7
Schedule of tumour inoculations

| | | | | | Sex of |
|---|---|---|---|---|---|
| Cell Line | Cancer Type | Cell Amount | Inoculation Site | Mouse Strain | Mouse |
| H22 | Liver | 1 × 10⁶ | Right front flank | Balb/c | Female |
| EMT-6 | Breast | 5 × 10⁵ | Right rear flank | Balb/c | Female |
| MC38 | Colon | 1 × 10⁶ | Right rear flank | C57BL/6 | Female |
| B16F | Melanoma | 2 × 10⁵ | Right rear flank | C57BL/6 | Female |
| Pan02 | Pancreatic | 3 × 10⁶ | Right front flank | C57BL/6 | Female |
| CT26 | Colon | 5 × 10⁵ | Right rear flank | Balb/c | Female |
| LL/2 | Lung | 3 × 10⁵ | Right rear flank | C57BL/6 | Female |
| A20 | Lymphoma | 5 × 10⁵ | Right rear flank | Balb/c | Female |
| Hepa1-6 | Liver | 5 × 10⁶ | Right front flank | C57BL/6 | Female |
| Renca | Kidney | 1 × 10⁶ | Right rear flank | Balb/c | Female |
| B16BL6 | Melanoma | 2 × 10⁵ | Right rear flank | C57BL/6 | Female |
| RM-1 | Prostate | 1 × 10⁶ | Right rear flank | C57BL/6 | Male |

After tumour inoculation, the animals were checked daily for morbidity or mortality. During routine monitoring, the animals were checked for any effects of tumour growth and treatments on behaviour such as mobility, food and water consumption, body weight gain/loss (body weights were measured twice per week after randomisation), eye/hair matting and any other abnormalities. Mortality and observed clinical signs were recorded for individual animals in detail.

The tumour volumes were measured twice per week after randomisation in two dimensions using a caliper, and the volume was expressed in mm³ using the formula: “V=(L×W×W)/2, where V is the tumour volume, L is the tumour length (the longest tumour dimension), and W is the tumour width (the longest tumour dimension perpendicular to L)”.

Randomisation was carried our when the mean tumour size reached approximately 65-100 mm³ and 20 mice were enrolled for each model. All animals were randomly assigned into two study groups with 10 mice in each group. Dosing of the compound started the day after randomisation. Randomisation was performed based on “Matched distribution” method (StudyDirector™ software, version 3.1.399.19). The date of randomisation was denoted as Day 0.

Mice were dosed according to the following dosing schedule:

TABLE 8
Dosing schedule.

| | | | | Dose | | Dosing |
|---|---|---|---|---|---|---|
| Group | Com- | | Dose | Concen- | | Frequency |
| (No per | pound | Dose | Volume | tration | Route/ | (BID/QD/ |
| group) | ID | (mg/kg) | (mL/kg) | (mg/ml) | N/A | TID) |
| 1(10) | PBS | 0 | 10 | 0 | i.p. | 3 doses; |
| | (Vehicle) | | | | | Day 1, Day |
| | | | | | | 3, and Day 5 |
| 2(10) | ATL5170 | 10 | 10 | 1 | i.p. | 3 doses; |
| | | | | | | Day 1, Day |
| | | | | | | 3, and Day 5 |

Intraperitoneal (i.p) maximum volume was 20 ml/kg.

Information regarding CFH expression in cell lines was determined by reference to the following publication/database: Zexian Zeng, Cheryl J Wong, Lin Yang, Nofal Ouardaoui, Dian Li, Wubing Zhang, Shengqing Gu, Yi Zhang, Yang Liu, Xiaoqing Wang, Jingxin Fu, Liye Zhou, Boning Zhang, Sarah Kim, Kathleen B Yates, Myles Brown, Gordon J Freeman, Ravindra Uppaluri, Robert Manguso, X Shirley Liu, TISMO: syngeneic mouse tumor database to model tumor immunity and immunotherapy response, Nucleic Acids Research, Volume 50, Issue D1, 7 Jan. 2022, Pages D1391-D1397, doi.org/10.1093/nar/gkab804.

Mutation burden and macrophage infiltration into tumours was estimated by reference to publicly available references.

The reference CFH expression (mRNA) data was obtained from a public database, OncoExpress™, provided by Crown Bioscience oncoexpress.crownbio.com and used with permission

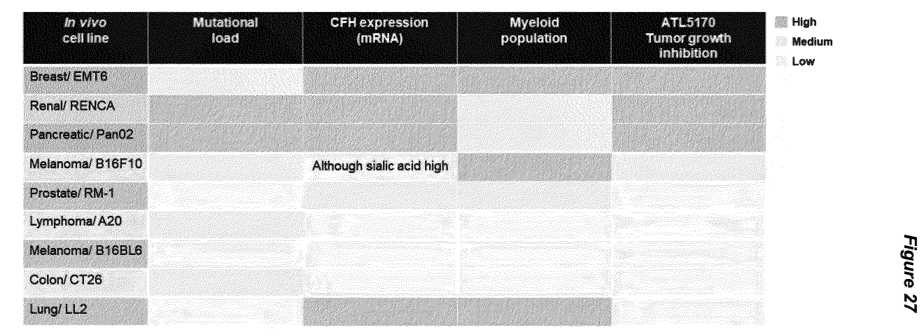

ATL5170 strongly inhibited tumour growth in a selected group of tumour models and tumour inhibition was associated with high CFH expression, high mutational burden, and medium to high immune cell infiltration into the tumour (FIG. 27). Based on these data observed in mice, it seems likely that ATL5170 will be particularly useful in treating cancer patients presenting with one or more of these markers (high CFH, high mutational load, and/or high tumour immune cell infiltration).

In view of the association between immune cell infiltration into the tumour and ATL5170-mediated tumour growth inhibition, combining the ATL5170 with tumour microenvironment-modulating therapies may be beneficial in some cancer patients.

In conclusion, ATL5170 shows strong anti-tumour activity in vivo, particularly in tumours with high CFH expression and myeloid infiltration.

### Example 6—Anti-CFH ATL5170 Causes Increase in Proapoptotic Signalling

Blocking the actions of CFH against surface bound C3b allows initiation of the complement cascade. This is relevant in tumours known to have elevated levels of CFH, which uses CFH to prevent attack against cancer cells which have accumulated surface bound levels of C3b. Enabling complement mediated attack will result in cell death (activation of apoptosis and necrosis).

To demonstrate the ability of ATL5170 to induce proapoptotic signalling in this way, this process was recreated in a syngeneic mouse EMT6 breast carcinoma model.

Briefly, female C57BI/6 mice were inoculated with EMT6 tumour cells by subcutaneous deposition. When tumours reached 60-100 mm3 in volume, mice were randomized, and selected cohort of mice received treatment with ATL5170.

Upon conclusion of the trial, tumours were excised, and frozen tissue sections were prepared for histological analysis. For the analysis, tissue sections were fixed, washed, and incubated with staining solution for quantification of TUNEL (Abcam) positivity marking pro-apoptotic cells. TUNEL staining is a routine histochemical stain frequently used to identify DNA damage, such as that which is associated with induced cancer cell death following therapeutic application of potential medicines (Gamrekelshvili et al. 2007)

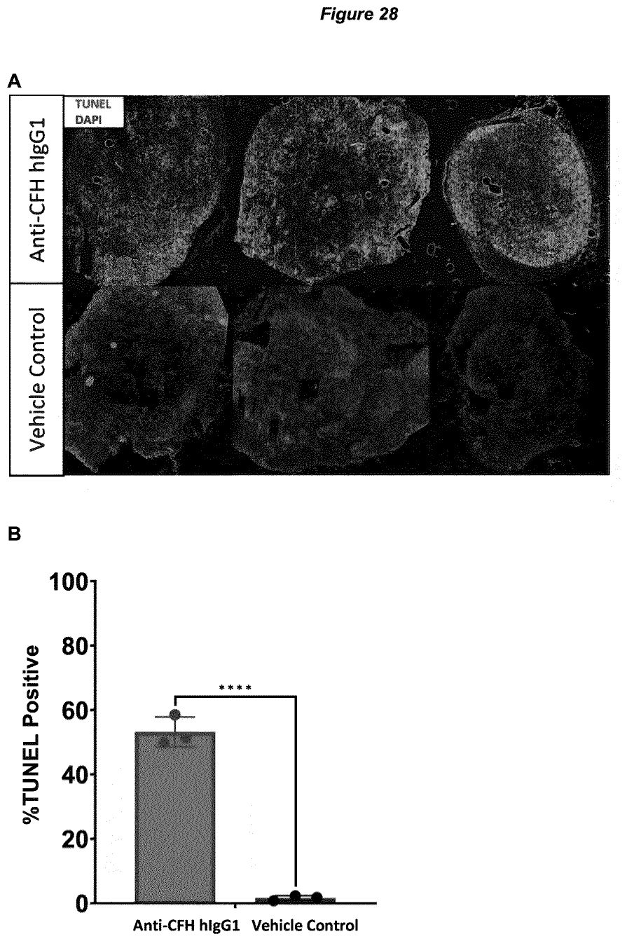

Imaging was carried out with the ImageXpress PICO imaging system (molecular devices) and quantified using CellReporterXpress imaging software, 3-channel cell scoring option. See FIG. 28A. Summary outputs were used to complete t-test analysis on slide sets, looking at positive percentage expression between ATL_5170 treated cohort and other treatment groups; n=3 for all cohorts.

Analysis of the percentage of TUNEL positive cells in tumour tissue taken from mice treated with ATL5170 revealed a significant increase in proapoptotic cells compared to vehicle control (FIG. 28B).

### Example 7—Inhibition of CFH with ATL5170 does not Cause Depletion of Systemic C3

To ensure that the mechanism of ATL5170 is focussed on cell surface associated CFH, systemic C3 levels were measured following dose with ATL5170 in the EMT6 murine syngeneic tumour model.

Briefly, female C57BI/6 mice were inoculated with EMT6 tumour cells by subcutaneous deposition. When tumours reached 60-100 mm3 in volume, mice were randomized, and selected cohort of mice received treatment with ATL5170 or with vehicle control

Levels of systemic C3 were determined by ELISA (Mouse Complement C3 ELISA Kit, Abcam) in serum samples collected post termination, as per the manufacturer's protocol. 100 ul diluted standard or sample were incubated for in a pre-coated microplate. Wells were then washed four times with wash buffer, prior to addition of enzyme-antibody conjugate. The plate was incubated for 20 minutes and then washed five times with wash buffer. TMB substrate was added, and the reaction stopped after 10 minutes. Absorbance (450 nm) was determined and the concentration of C3 interpolated from the standard curve.

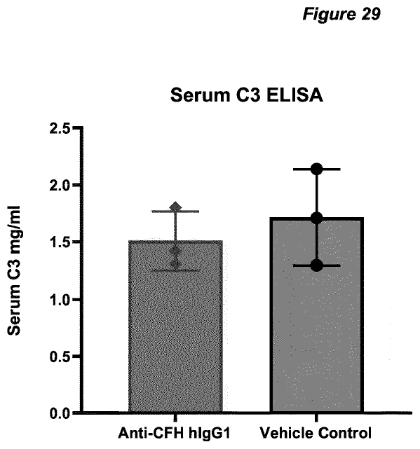

The resulting analysis confirmed that systemic serum C3 levels remained unaltered compared to vehicle control following dose regimen with ATL5170 (FIG. 29).

### SUMMARY

ATL5170 is a novel anti-CFH antibody which displays:
Significantly improved thermostability compared to germlined ATL4717 and 7 degrees higher Tₘ than ATL4715 benchmark, as measured by both protein melting curve and heat challenge ELISA.
Significantly improved production yield compared to original non-germlined ATL4717.
Capable of specific binding to cell-associated CFH, but not free circulating CFH
Capable of binding to CFH family proteins: reduced CFHR1, reduced CFHR2, but not CFHR3, 4 or 5.
A broad immune activation profile, which supports an anti-tumour mechanism of action.
Strong anti-tumour activity in vivo.

### REFERENCES

A number of publications are cited above in order to more fully describe and disclose the invention and the state of the art to which the invention pertains. Full citations for these references are provided below. The entirety of each of these references is incorporated herein.
1. Ricklin D, Hajishengallis G, Yang K, Lambris J D. Complement: a key system for immune surveillance and homeostasis. Nat Immunol (2010) 11:785-97. doi: 10.1038/ni. 1923
2. Schmidt C Q, Lambris J D, Ricklin D. Protection of host cells by complement regulators. Immunol Rev (2016) 274:152-71. doi: 10.1111/imr.12475)
3. Kazatchkine M D, Fearon D T, Austen K F. Human alternative complement pathway: membrane-associated sialic acid regulates the competition between B and beta1 H for cell-bound C3b. J Immunol (1979) 122:75-81.
4. Weiler J M, Daha M R, Austen K F, Fearon D T. Control of the amplification convertase of complement by the plasma protein beta1H. Proc Natl Acad Sci USA (1976) 73:3268-72. doi: 10.1073/pnas.73.9.3268
5. Harrison R A, Lachmann P J. The physiological breakdown of the third component of human complement. Mol Immunol (1980) 17:9-20. doi: 10.1016/0161-5890 (80) 90119-4
6. Brader M L, Estey T, Bai S, Alston R W, Lucas K K, Lantz S, Landsman P, Maloney K M. Examination of thermal unfolding and aggregation profiles of a series of developable therapeutic monoclonal antibodies. Mol Pharm. 2015; 12:1005-1017. doi: 10.1021/mp400666b.
7. Garber E, Demarest S J. A broad range of Fab stabilities within a host of therapeutic IgGs. Biochem Biophys Res Commun. 2007; 355:751-757. doi: 10.1016/j.bbrc.2007.02.042.
8. Vermeer A W, Norde W. The thermal stability of immunoglobulin: unfolding and aggregation of a multi-domain protein. Biophys J. 2000; 78:394-404. doi: 10.1016/S0006-3495 (00) 76602-1.
9. Vermeer A W, Norde W, van Amerongen A. The unfolding/denaturation of immunogammaglobulin of isotype 2b and its F (ab) and F (c) fragments. Biophys J. 2000; 79:2150-2154. doi: 10.1016/S0006-3495 (00) 76462-9.
10. Moore S R, Smrithi S M, Cortes C Ferreira V P. Hijacking Factor H for Complement Immune Evasion. Front. Immunol., 25 Feb. 2021| doi.org/10.3389/fimmu.2021.602277
11. Galson J D, et al. Deep sequencing of B cell receptor repertoires from COVID-19 patients reveals strong convergent immune signatures. Front. Immunol., 15 Dec. 2020| https://doi.org/10.3389/fimmu.2020.605170.
12. Whaley K, Ruddy S. Modulation of the alternative complement pathways by beta 1H globulin. J Exp Med., 2 Nov. 1976. doi: 10.1084/jem. 144.5.1147.
13. Michael K. Pangburn, Kerry L. W. Pangburn, Vesa Koistinen, Seppo Meri, Ajay K. Sharma. The Journal of Immunology May 1, 2000, 164 (9) 4742-4751; DOI: 10.4049/jimmunol. 164.9.4742.
14. Daniel Ajona, Zafira Castaño, Mercedes Garayoa, Enrique Zudaire, Maria J. Pajares, Alfredo Martinez, Frank Cuttitta, Luis M. Montuenga, Ruben Pio; Expression of Complement Factor H by Lung Cancer Cells: Effects on the Activation of the Alternative Pathway of Complement. Cancer Res 1 Sep. 2004; 64 (17): 6310-6318. doi.org/10.1158/0008-5472.
15. Karolina I. Smolag, Christine M. Mueni, Karin Leandersson, Karin Jirström, Catharina Hagerling, Matthias Morgelin, Paul N. Barlow, Myriam Martin & Anna M. Blom (2020) Complement inhibitor factor H expressed by breast cancer cells differentiates CD14+ human monocytes into immunosuppressive macrophages, Oncolmmunology, 9:1, DOI: 10.1080/2162402X.2020.1731135.
16. Nissila et al. Complement Factor H and Apolipoprotein E Participate in Regulation of Inflammation in THP-1 Macrophages. Front. Immunol., 21 Nov. 2018| https://doi.org/10.3389/fimmu.2018.02701
17. Yoon, Y. H., Hwang, H. J., Sung, H. J., Heo, S. H., Kim, D. S., Hong, S. H., Lee, K. H., & Cho, J. Y. (2019). Upregulation of Complement Factor H by SOCS-1/3-STAT4 in Lung Cancer. Cancers, 11 (4), 471. doi.org/10.3390/cancers11040471
18. Duan, Z., Luo, Y. Targeting macrophages in cancer immunotherapy. Sig Transduct Target Ther 6, 127 (2021). doi.org/10.1038/s41392-021-00506-6
19. Gamrekelashvili J, Krüger C, von Wasielewski R, Hoffmann M, Huster K M, Busch D H, Manns M P, Korangy F, Greten T F. Necrotic tumor cell death in vivo impairs tumor-specific immune responses. J Immunol. 2007 Feb. 1; 178 (3): 1573-80.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An isolated antibody or antibody fragment thereof which specifically binds to Complement Factor H (CFH) protein or a fragment thereof, wherein the antibody comprises:
a. a heavy chain variable domain (VH) with the following CDRs:
i. HCDR1 comprising amino acid sequence SEQ ID NO:12,
ii. HCDR2 comprising amino acid sequence SEQ ID NO:13, and
iii. HCDR3 comprising amino acid sequence SEQ ID NO:17 or SEQ ID NO:3,
and
b. a light chain variable domain (VL) with the following CDRs:
i. LCDR1 comprising amino acid sequence SEQ ID NO:14,
ii. LCDR2 comprising amino acid sequence SEQ ID NO:5, and
iii. LCDR3 comprising amino acid sequence SEQ ID NO:6.

2. The isolated antibody or antibody fragment thereof according to claim 1, comprising one or more framework substitutions, wherein the substitutions are selected from VH domain substitutions L50P, S70G, and L123Q, and/or VL substitutions L11Q and E68V, wherein position numbering is IMGT.

3. The isolated antibody or antibody fragment thereof according to claim 1, wherein:
the heavy chain variable domain comprises amino acid sequence SEQ ID NO:18, and/or the light chain variable domain comprises amino acid sequence SEQ ID NO:19.

4. The isolated antibody or antibody fragment thereof according to claim 1, wherein the antibody is IgG1.

5. The isolated antibody or antibody fragment thereof according to claim 1, wherein the heavy chain has at least 95% sequence identity with SEQ ID NO:22, and/or the light chain has at least 95% sequence identity with SEQ ID NO:23.

6. The isolated antibody or antibody fragment thereof according to claim 1, wherein the antibody or fragment thereof: (i) has a less than 50% reduction in binding to reduced CFH after a heat challenge at 60° C. for 8 minutes or 70° C. for one hour, optionally wherein binding is detected by phage ELISA; and/or (ii) stimulates IL-8 secretion in a THP-1 cytokine release assay; and/or (iii) does not bind CFHR5.

7. The isolated antibody or antibody fragment thereof according to claim 1, wherein the antibody or fragment thereof binds to reduced CFH, reduced CFHR1, and/or reduced CFHR2.

8. The isolated antibody or antibody fragment thereof according to claim 1, wherein the antibody or fragment thereof: (i) does not bind to soluble CFH; and/or (ii) does not reduce systemic C3 levels following administration of the antibody or fragment to a mouse cancer model, optionally wherein the mouse cancer model is the EMT6 murine syngeneic tumour model and/or wherein levels of systemic C3 are determined by ELISA.

9. The isolated antibody or antibody fragment thereof according to claim 1, wherein the antibody or fragment thereof: (i) induces differentiation of monocytes into an activated macrophage state, optionally wherein induction into an activated macrophage is detected by an increase in the frequency of CD11b+CD14−CD45+ cells and a decrease in frequency of CD14+CD11b−CD45+ cells and/or an increase level of IL-6, TNF-α, and IL-1β, upon treatment of monocytes with the antibody or fragment thereof; and/or (ii) induces differentiation of monocytes into an activated macrophage state in a FcR independent manner; and/or (iii) reduces phagocytosis of pHRodo labelled bacteria by macrophages; and/or (iv) increases the level of secretion of IL-6, TNF-α, and IL-1β by macrophages; and/or increases CD4+ activation and/or proliferation, optionally wherein increased CD4+ activation is determined by a change in shape and granularity indicated by a shift in forward and side scatter measured by flow cytometry upon exposure of CD4 T cells to the antibody or fragment thereof; activates CD8 T cells, optionally wherein activation of CD8 T cells is determined by an increased expression of CD69 on CD8 T cells, when co-culturing PBMCs and PDAC10.02 tumour cells in the presence of the antibody or antibody fragment.

10. The isolated antibody or antibody fragment thereof according to claim 1, wherein the antibody or fragment thereof: (i) inhibits tumour growth in vivo, optionally wherein the antibody or antibody fragment inhibits tumour growth in a EMT6-BALB/c syngeneic mouse tumour model; and/or (ii) induces proapoptotic signalling, optionally wherein induction of proapoptotic signalling is determined as an increase in the percentage of TUNEL positive cells in tumour tissue taken from EMT6 mice treated with the antibody or fragment.

11. The isolated antibody or antibody fragment according to claim 1, wherein the antibody or fragment: (i) binds to the Sushi 19 (SCR19) domain of CFH.

12.-14. (canceled)

15. A method of treating a disease or disorder, comprising administering a therapeutically effective amount of an isolated antibody or antibody fragment thereof according to claim 1.

16.-17. (canceled)

18. The method according to claim 12, wherein the disease or disorder is cancer, optionally wherein the cancer is a cancer with high CFH expression and/or high immune cell infiltration and/or high mutational burden compared to a control level.

19. The method according to claim 12, wherein the disease or disorder is an infectious disease or disorder.

20. The method according to claim 12, wherein the treatment comprises the administration of a further therapeutic agent, simultaneously or sequentially with the isolated antibody or antibody fragment.

21. The method according to claim 15, wherein the further immunotherapeutic agent is an immune checkpoint inhibitor.

22. A method of increasing complement dependent lysis of a cell, increasing C3b and/or C3d deposition on a cell, and/or inhibiting CFH binding to C3b in a cell, and/or activating an immune cell, the method comprising contacting the cell with an antibody or antibody fragment thereof according to claim 1.

23.-26. (canceled)

27. The isolated antibody or antibody fragment thereof according to claim 1, wherein:
the heavy chain variable domain comprises amino acid sequence SEQ ID NO:15, and/or the light chain variable domain comprises amino acid sequence SEQ ID NO:16.

28. The isolated antibody or antibody fragment thereof according to claim 1, wherein the antibody or fragment thereof does not bind to CFHR3, CFHR4, and/or CFHR5.

29. The isolated antibody or antibody fragment according to claim 1, wherein the antibody or fragment: (i) binds to an epitope within CFH SCR19 domain comprising or consisting of 4-8 contiguous nucleic acids of SEQ ID NO:25; and/or (ii) competes, blocks, or sterically hinders antibodies capable of binding an epitope comprising or consisting of 4-8 contiguous nucleic acids of SEQ ID NO:25.
